(19) United States
(12) Patent Application Publication
(10) Pub. No.: US 2023/0173092 A1
(43) Pub. Date: Jun. 8, 2023

(54) ANTIBODY-DRUG CONJUGATES

(71) Applicant: SPIREA LIMITED, Harrogate (GB)

(72) Inventors: Myriam OUBERAI, Harrogate (GB); Neil SIM, Durham (GB); James FLEMING, Durham (GB); Nicolas CAMPER, Cambridge (GB); Mark FRIGERIO, Cambridge (GB)

(73) Assignee: SPIREA LIMITED, Harrogate (GB)

(21) Appl. No.: 17/999,815

(22) PCT Filed: May 26, 2021
(86) PCT No.: PCT/GB2021/051285

§ 371 Date: Nov. 23, 2022

(30) Foreign Application Priority Data
May 27, 2020 (GB) 2007926.5

(51) Int. Cl. A61K 47/68; A61P 35/00; C07K 16/00

(52) U.S. Cl. CPC A61K 47/6883; A61K 47/6803; A61K 47/6889; A61P 35/00; C07K 16/00; C07K 2317/73; C07K 2317/92

(57) ABSTRACT

The present invention relates to antibody-drug conjugates comprising (i) an antibody or antigen-binding fragment thereof, (ii) a polymer comprising a particular repeat unit comprising an amino acid derivative, which is covalently bound to one or more biologically active moieties, such as small molecule drugs, optionally via a linker, and (iii) a polymer-antibody linker moiety which is covalently bound to both the polymer and the antibody or antigen-binding fragment thereof. Additionally, the present invention relates to pharmaceutical compositions comprising the antibody-drug conjugates and to use of the antibody-drug conjugates in medicine.

### FIELD OF THE INVENTION

The present invention relates to antibody-drug conjugates comprising (i) an antibody or antigen-binding fragment thereof, (ii) a polymer comprising a particular repeat unit comprising an amino acid derivative, which is covalently bound to one or more biologically active moieties, such as small molecule drugs, optionally via a linker, and (iii) a polymer-antibody linker moiety which is covalently bound to both the polymer and the antibody or antigen-binding fragment thereof. Additionally, the present invention relates to pharmaceutical compositions comprising the antibody-drug conjugates and to use of the antibody-drug conjugates in medicine.

### BACKGROUND INFORMATION

Antibody drug conjugates (ADCs) are a class of highly potent biopharmaceutical drugs, which have various therapeutic uses. For example, in the oncology field, ADCs can be used to target cancerous cells using an antibody on which a cytotoxic drug is attached via a linker. Despite these benefits, the development of ADCs has been limited due to the low drug-to-antibody ratios (DARs) of 3-4 that can be typically achieved. Often, with conventional ADCs, only one drug can be attached to the antibody per linker. This restriction limits the therapeutic index of ADCs and the range of drugs that can be used in ADCs, since only highly cytotoxic drugs can be employed. This also increases the prevalence of adverse reactions in patients. In addition, attempts to date to increase the DAR have resulted in aggregation of the ADC, rendering it ineffective.

There is therefore a need for new ADCs which can support a high DAR but which also have desirable physicochemical properties, such as high aqueous solubility and stability.

### SUMMARY OF THE INVENTION

The present invention provides an ADC containing a specific polymeric linker, which enables good stability and high solubility in aqueous solution. The specific polymeric linker used in the present invention can also support a high DAR, and is able to conjugate many different biologically active molecules (typically, 4 or more, 8 or more, preferably 12 or more, yet more preferably 16 or more, and most preferably up to 20 or more biologically active molecules) to a single antibody. Such a high DAR enables an improved therapeutic index.

Furthermore, the specific polymer used in the ADCs of the present invention may also enable the release rate of the biologically active molecules from the conjugate to be controlled. This release rate depends on the degradation of the covalent polymer-drug or linker-drug bonds within the ADC. Different types of covalent linkage will hydrolyse under different conditions of (e.g.) pH, enzyme.

The specific polymer used in the ADCs of the present invention also enables multiple different types of drug moiety to be conjugated to the polymer. That can be useful, in particular, in achieving targeted combination therapy using two or more active agents. Combination therapies are particularly useful in oncology and the treatment of infectious diseases. The drugs used in combination therapies often have complimentary modes of action and/or have additive or synergistic therapeutic effects. The treatment protocols employing multiple drugs are, however, invariably complicated and intensive. Frequent drug dosing and concomitant administration of several different drugs at a given point in time is commonplace. Such complicated protocols tend to have lower patient compliance and tolerance than more straightforward protocols. The ability to conjugate multiple drugs to a single antibody with high DAR and favourable physicochemical properties therefore offers new opportunities in combination therapies.

The specific polymer used in the ADCs of the present invention is also surprisingly found to prevent agglomeration/aggregation of the ADCs in solution, even when the DAR is high, and to have improved serum stability compared to control ADCs having a different polymer backbone/linker.

The present invention accordingly provides an antibody-drug conjugate comprising:
(i) an antibody or antigen-binding fragment thereof;
(ii) a polymer comprising a repeat unit of Formula (I):

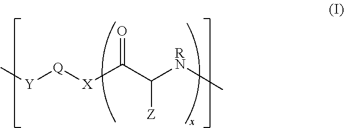

wherein:
X is selected from O, NH, NR^{A} and S;
Y is selected from C═O, C═NH, C═NR^{A} and C═S;
R is hydrogen or C₁₋₂₀ hydrocarbyl;
R^{A} is C₁₋₂₀ hydrocarbyl;
each Q is independently selected from —CH₂(NMe(C═O)CH₂)ₒ—, -T¹O(CH₂C₂O)ₛT²- and -T¹O(CH₂CH₂C₂O)ₛT²-, wherein T¹ is selected from a divalent methylene, ethylene, propylene or butylene radical, and T² is selected from a divalent methylene, ethylene, propylene or butylene radical;
o is an integer from 0 to 100;
s is an integer from 0 to 150;
x is an integer from 1 to 6; and
each Z is independently selected from a group of formula (i), (ii), (iii), (iv) or (v):

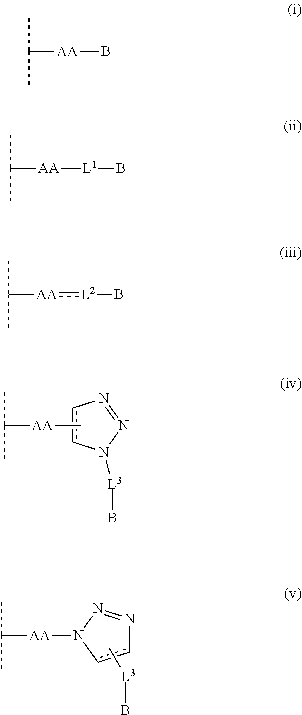

wherein,
when Z is a group of formula (i) or (ii):
   -AA- is a divalent moiety such that -AA-H represents the side chain of an amino acid;
   each L¹ is a linker group; and
   each B is a biologically active moiety;
when Z is a group of formula (iii):
   -AA= is a trivalent moiety such that -AA=O represents the side chain of an amino acid;
   each L² is a linker group;
   each dashed line represents a bond which is either present or absent; and
   each B is a biologically active moiety;
when Z is a group of formula (iv):
   -AA- is a divalent moiety such that -AA-CH═CH₂ or -AA-CCH represents the side chain of an amino acid;
   each L³ is a linker group;
   each dashed line represents a bond which is either present or absent; and
   each B is a biologically active moiety; and
when Z is a group of formula (v):
   -AA- is a divalent moiety such that -AA-N₃ represents the side chain of an amino acid;
   each L³ is a linker group;
   each dashed line represents a bond which is either present or absent; and
   each B is a biologically active moiety; and
(iii) a polymer-antibody linker which is covalently bonded to both the antibody and the polymer.

In another aspect, the present invention also provides a pharmaceutical composition comprising an antibody-drug conjugate according to the invention, and a pharmaceutically acceptable excipient.

The present invention further provides an antibody-drug conjugate according to any the invention for use in the treatment of a disease or condition in a patient in need thereof.

The present invention further provides a method of treating a disease or condition as defined herein in a human patient, wherein said method comprises administration of at least one antibody-drug conjugate according to the invention to a patient in need thereof.

The present invention further provides the use of an antibody-drug conjugate according to the invention for the manufacture of a medicament for the treatment of a disease or condition as defined herein in a patient.

The present invention further provides a targeting agent-drug conjugate comprising:
(i) a targeting agent;
(ii) a polymer comprising a repeat unit of Formula (I); and
(iii) a polymer-targeting agent linker which is covalently bonded to both the targeting agent and the polymer.

### BRIEF DESCRIPTION OF THE DRAWINGS

FIG. 1: ¹H-NMR spectrum of building block (3) at 400 MHz and 298 K in CDCl₃.

FIG. 2: Mass spectrum of polymer (1).

FIG. 3: Mass spectrum of polymer (4).

FIG. 4: LC-MS spectrum of MMAE reagent (5).

FIG. 5: LC-MS spectrum of MMAE reagent (5).

FIG. 6: RP-UPLC spectrum of polymer-drug conjugate (6) at 214 nm.

FIG. 7: LC-MS spectrum of polymer-drug conjugate (6).

FIG. 8: Graph of tumour volume against time to show the in vivo anti-tumour efficacy of the MMAE ADC in NCI-N87 human gastric cancer CDX model. ADC=MMAE ADC produced as described in Example 3.

FIG. 9: LC-MS analysis of polymer (7).

FIG. 10: LC-MS analysis of polymer (8).

FIG. 11: RP-HPLC (λ=214 nm) analysis of SN-38 polymer conjugate (11).

FIG. 12: LC-MS analysis of SN-38 polymer conjugate (11).

FIG. 13: RP-HPLC (λ=214 nm) analysis of SN-38 polymer conjugate (13).

FIG. 14: LC-MS analysis of SN-38 polymer conjugate (13).

### DETAILED DESCRIPTION OF THE INVENTION

Definitions

As used herein, the term “polymer” refers to a compound comprising repeating units. Polymers usually have a polydispersity of greater than 1. Polymers generally comprise a backbone, side chains and termini. The backbone is the linear chain to which all side chains are pendant. The side chains are the groups that are pendant to the backbone or branch off the backbone. The termini are the ends of the backbone.

As used herein, the term “biologically active moiety” refers to any moiety that is derived from a biologically active molecule by abstraction of a hydrogen radical. A “biologically active molecule” is any molecule capable of inducing a biochemical response when administered in vivo. Typically, the biologically active molecule is capable of producing a local or systemic biochemical response when administered to an animal (or, preferably, a human); preferably the local or systemic response is a therapeutic activity. Preferred examples of biologically active molecules include drugs, peptides, proteins, peptide mimetics, antibodies, antigens, DNA, RNA, mRNA, small interfering RNA, small hairpin RNA, microRNA, PNA, foldamers, carbohydrates, carbohydrate derivatives, non-Lipinski molecules, synthetic peptides and synthetic oligonucleotides, and most preferably small molecule drugs.

As used herein, the term “small molecule drug” refers to a chemical compound which has known biological effect on an animal, such as a human. Typically, drugs are chemical compounds which are used to treat, prevent or diagnose a disease. Preferred small molecule drugs are biologically active in that they produce a local or systemic effect in animals, preferably mammals, more preferably humans. The small molecule drug may be referred to as a “drug molecule” or “drug”. Typically, the drug molecule has M_{W} less than or equal to about 5 kDa. Preferably, the drug molecule has M_{W} less than or equal to about 1.5 kDa. A more complete, although not exhaustive, listing of classes and specific drugs suitable for use in the present invention may be found in “Pharmaceutical Substances: Syntheses, Patents, Applications” by Axel Kleemann and Jurgen Engel, Thieme Medical Publishing, 1999 and the “Merck Index: An Encyclopedia of Chemicals, Drugs, and Biologicals”, edited by Susan Budavari et al., CRC Press, 1996, both of which are incorporated herein by reference in their entirety.

As used herein, the term “peptides” refers to biologically occurring or synthetic short chains of amino acid monomers linked by peptide (amide) bonds. The covalent chemical bonds are formed when the carboxyl group of one amino acid reacts with the amino group of another. The shortest peptides are dipeptides, consisting of 2 amino acids joined by a single peptide bond, followed by tripeptides, tetrapeptides, etc. A polypeptide is a long, continuous, and unbranched peptide chain. Hence, peptides fall under the broad chemical classes of biological oligomers and polymers, alongside nucleic acids, oligosaccharides and polysaccharides, etc.

As used herein, the term “amino acid” refers to any natural or synthetic amino acid, that is, an organic compound comprising carbon, hydrogen, oxygen and nitrogen atoms, and comprising both amino (—NH₂) and carboxylic acid (—COOH) functional groups. Typically, the amino acid is an α-, β-, γ- or δ-amino acid. The amino acid may be one of the twenty-two naturally occurring proteinogenic α-amino acids. Alternatively, the amino acid is a synthetic amino acid selected from α-Amino-n-butyric acid, Norvaline, Norleucine, Alloisoleucine, t-leucine, α-Amino-n-heptanoic acid, Pipecolic acid, α,β-diaminopropionic acid, α,γ-diaminobutyric acid, Ornithine, Allothreonine, Homocysteine, Homoserine, β-Alanine, β-Amino-n-butyric acid, β-Aminoisobutyric acid, γ-Aminobutyric acid, α-Aminoisobutyric acid, isovaline, Sarcosine, N-ethyl glycine, N-propyl glycine, N-isopropyl glycine, N-methyl alanine, N-ethyl alanine, N-methyl β-alanine, N-ethyl β-alanine, isoserine, α-hydroxy-γ-aminobutyric acid, Homonorleucine, O-methyl-homoserine, O-ethyl-homoserine, selenohomocysteine, selenomethionine, selenoethionine, Carboxyglutamic acid, Hydroxyproline, Hypusine, Pyroglutamic acid, aminoisobutyric acid, dehydroalanine, β-alanine, γ-Aminobutyric acid, δ-Aminolevulinic acid, 4-Aminobenzoic acid, citrulline, 2,3-diaminopropanoic acid, 3-aminopropanoic acid, hydroxytryptophan, selenohomocysteine, α-aminoglycine and diaminoacetic acid, 2,3-diaminopropionic acid, α,γ-diaminobutyric acid, amino-2-keto-butyric acid, 4-acetylphenylalanine and formylglycine, azidolysine, azidoornithine, azidonorleucine, azidoalanine, azidohomoalanine, 4-azidophenylalanine and 4-azidomethylphenylalanine, homoallylglycine, 4-ethynylphenylalanine, 4-propargyloxyphenylalanine, propargylglycine, 4-(2-propynyl)proline, 2-amino-6-({[(1R,8S)-bicyclo[6.1.0]non-4-yn-9-ylmethoxy]carbonyl}amino)hexanoic acid and homopropargylglycine. An amino acid which possess a stereogenic centre may be present as a single enantiomer or as a mixture of enantiomers (e.g. a racemic mixture). Preferably, if the amino acid is an α-amino acid, the amino acid has L stereochemistry about the α-carbon stereogenic centre.

As used herein, the term “proteins” refers to biological molecules comprising polymers of amino acid monomers which are distinguished from peptides on the basis of size, and as an arbitrary benchmark can be understood to contain approximately 50 or more amino acids. Proteins consist of one or more polypeptides arranged in a biologically functional way, often bound to ligands such as coenzymes and cofactors, or to another protein or other macromolecule (DNA, RNA, etc.), or to complex macromolecular assemblies.

As used herein, the term “peptide mimetics” refers to small protein-like chains designed to mimic a peptide. They typically arise either from modification of an existing peptide, or by designing similar systems that mimic peptides, such as peptoids and β-peptides. Irrespective of the approach, the altered chemical structure is designed to advantageously adjust the molecular properties such as, stability or biological activity. This can have a role in the development of drug-like compounds from existing peptides. These modifications involve changes to the peptide that will not occur naturally (such as altered backbones and the incorporation of non-natural amino acids).

As used herein, the term “mRNA” refers to messenger RNA, a family of RNA molecules that convey genetic information from DNA to the ribosome, where they specify the amino acid sequence of the protein products of gene expression. Following transcription of primary transcript mRNA (known as pre-mRNA) by RNA polymerase, processed, mature mRNA is translated into a polymer of amino acids: a protein. As in DNA, mRNA genetic information is in the sequence of nucleotides, which are arranged into codons consisting of three bases each. Each codon encodes for a specific amino acid, except the stop codons, which terminate protein synthesis. This process of translation of codons into amino acids requires two other types of RNA: transfer RNA (tRNA), that mediates recognition of the codon and provides the corresponding amino acid, and ribosomal RNA (rRNA), that is the central component of the ribosome's protein-manufacturing machinery.

As used herein, the term “small interfering RNA” (siRNA) refers to a class of double-stranded RNA molecules, 20-25 base pairs in length. siRNA plays many roles, but it is most notable in the RNA interference (RNAi) pathway, where it interferes with the expression of specific genes with complementary nucleotide sequences. siRNA functions by causing mRNA to be broken down after transcription, resulting in no translation. siRNA also acts in RNAi-related pathways, e.g. as an antiviral mechanism or in shaping the chromatin structure of a genome.

As used herein, the term “small hairpin RNA” (shRNA) refers to an artificial RNA molecule with a tight hairpin turn that can be used to silence target gene expression via RNA interference (RNAi). Expression of shRNA in cells is typically accomplished by delivery of plasmids or through viral or bacterial vectors. shRNA is an advantageous mediator of RNAi in that it has a relatively low rate of degradation and turnover.

As used herein, the term “micro RNA” (miRNA) refers to a small non-coding RNA molecule (containing about 22 nucleotides) found in plants, animals, and some viruses, which functions in RNA silencing and post-transcriptional regulation of gene expression.

As used herein, the term “PNA” refers to peptide nucleic acid, an artificially synthesized polymer similar to DNA or RNA invented by Peter E. Nielsen (Univ. Copenhagen), Michael Egholm (Univ. Copenhagen), Rolf H. Berg (Risø National Lab), and Ole Buchardt (Univ. Copenhagen) in 1991. PNA's backbone is composed of repeating N-(2-aminoethyl)-glycine units linked by peptide bonds. The various purine and pyrimidine bases are linked to the backbone by a methylene bridge (—CH₂—) and a carbonyl group (—(C═O)—).

As used herein, the term “DNA” refers to deoxyribonucleic acid and derivatives thereof, the molecule that carries most of the genetic instructions used in the development, functioning and reproduction of all known living organisms and many viruses. Most DNA molecules consist of two biopolymer strands coiled around each other to form a double helix. The two DNA strands are known as polynucleotides since they are composed of simpler units called nucleotides. Each nucleotide is composed of a nitrogen-containing nucleobase—cytosine (C), guanine (G), adenine (A), or thymine (T)—as well as a monosaccharide sugar called deoxyribose and a phosphate group. The nucleotides are joined to one another in a chain by covalent bonds between the sugar of one nucleotide and the phosphate of the next, resulting in an alternating sugar-phosphate backbone. According to base pairing rules (A with T, and C with G), hydrogen bonds bind the nitrogenous bases of the two separate polynucleotide strands to make double-stranded DNA.

As used herein, the term “foldamer” refers to a discrete chain molecule or oligomer that folds into a conformationally ordered state in solution. They are artificial molecules that mimic the ability of proteins, nucleic acids, and polysaccharides to fold into well-defined conformations, such as helices and β-sheets. The structure of a foldamer is stabilized by non-covalent interactions between nonadjacent monomers.

As used herein, the term “carbohydrate” refers to biological molecule consisting of carbon (C), hydrogen (H) and oxygen (O) atoms, usually with a hydrogen:oxygen atom ratio of 2:1 (as in water); in other words, with the empirical formula Cₘ(H₂O)ₙ (where m could be different from n). Some exceptions exist; for example, deoxyribose, a sugar component of DNA, has the empirical formula C₅H₁₀O₄. Carbohydrates are technically hydrates of carbon; structurally it is more accurate to view them as polyhydroxy aldehydes and ketones. The term is most common in biochemistry, where it is a synonym of saccharide, a group that includes sugars, starch, and cellulose. The saccharides are divided into four chemical groups: monosaccharides, disaccharides, oligosaccharides, and polysaccharides.

As used herein, the term “non-Lipinski molecules” refers to molecules that do not conform to Lipinski's rule of five (also known as the Pfizer's rule of five or simply the Rule of five (RO5)), which is a rule of thumb to evaluate drug-likeness or to determine whether a chemical compound with a certain pharmacological or biological activity has properties that would make it a likely orally active drug in humans. The rule was formulated by Christopher A. Lipinski in 1997, based on the observation that most orally administered drugs are relatively small and moderately lipophilic molecules. The rule describes molecular properties important for a drug's pharmacokinetics in the human body, including their absorption, distribution, metabolism, and excretion (“ADME”). However, the rule does not predict if a compound is pharmacologically active.

As used herein, the term “acid-labile” refers to a bond which breaks in acidic conditions, e.g. a pH of <7.

As used herein, the term “direct bond” means that there are no intervening atoms. Thus, for example, a direct bond between a repeat unit and a drug means that a functional group of the drug is attached to an atom of the repeat unit, i.e. without the use of a linking group in-between.

As used herein, the term “C₁₋₂₀ hydrocarbyl” refers to any monovalent hydrocarbon radical comprising hydrogen and between 1 and 20 carbon atoms. Thus, hydrocarbyl groups consist of carbon and hydrogen. Examples of hydrocarbyl groups include alkyl, cycloalkyl, aryl, aralkyl, alkenyl, and alkynyl groups.

As used herein, the term “alkyl” refers to a linear or branched saturated monovalent hydrocarbon radical having the number of carbon atoms indicated in the prefix. Thus, the term “C₁₋₄ alkyl” refers to a linear saturated monovalent hydrocarbon radical of one to four carbon atoms or a branched saturated monovalent hydrocarbon radical of three or four carbon atoms, e.g. methyl, ethyl, n-propyl, iso-propyl, n-butyl, iso-butyl and tert-butyl. Preferably, an alkyl group is a C₁₋₂₀ alkyl group, more preferably a C₁₋₁₂ alkyl group, yet more preferably a C₁₋₈ alkyl group, and most preferably a C₁₋₄ alkyl group.

As used herein, the term “alkylene” refers to a linear saturated divalent hydrocarbon radical or a branched saturated divalent hydrocarbon radical having the number of carbon atoms indicated in the prefix, e.g. methylene, ethylene, propylene, 1-methylpropylene, 2-methylpropylene, butylene, pentylene, and the like. Preferably, an alkylene group is a C₁₋₂₀ alkylene group, more preferably a C₁₋₁₂ alkylene group, yet more preferably a C₁₋₈ alkylene group, and most preferably a C₁₋₄ alkylene group.

As used herein, the term “alkenyl” refers to a linear or branched saturated monovalent hydrocarbon radical having the number of carbon atoms indicated in the prefix and containing at least one double bond. Thus, the term “C₂₋₆ alkenyl” refers to a linear saturated monovalent hydrocarbon radical of two to six carbon atoms having at least one double bond, or a branched saturated monovalent hydrocarbon radical of three to six carbon atoms having at least one double bond, e.g. ethenyl, propenyl, 1,3-butadienyl, (CH₂)₂CH═C(CH₃)₂, CH₂CH═CHCH(CH₃)₂, and the like. Preferably, an alkenyl group is a C₂₋₂₀ alkenyl group, more preferably a C₂₋₁₂ alkenyl group, yet more preferably a C₂₋₈ alkenyl group, and most preferably a C₂₋₄ alkenyl group.

As used herein, the term “alkenylene” refers to a linear saturated divalent hydrocarbon radical or a branched saturated divalent hydrocarbon radical having the number of carbon atoms indicated in the prefix and containing at least one double bond, e.g. ethenylene, propenylene, 1-methylpropenylene, 2-methylpropenylene, butenylene, pentenylene, and the like. Preferably, an alkenylene group is a C₂₋₂₀ alkenylene group, more preferably a C₂₋₁₂ alkenylene group, yet more preferably a C₂₋₈ alkenylene group, and most preferably a C₂₋₄ alkenylene group.

As used herein, the term “alkynyl” refers to a linear or branched saturated monovalent hydrocarbon radical having the number of carbon atoms indicated in the prefix and containing at least one triple bond. Thus, the term “C₂₋₆ alkynyl” refers to a linear saturated monovalent hydrocarbon radical of two to six carbon atoms having at least one triple bond, or a branched saturated monovalent hydrocarbon radical of four to six carbon atoms having at least one double bond, e.g. ethynyl, propynyl, and the like. Preferably, an alkynyl group is a C₂₋₂₀ alkynyl group, more preferably a C₂₋₁₂ alkynyl group, yet more preferably a C₂₋₈ alkynyl group, and most preferably a C₂₋₄ alkynyl group.

As used herein, the term “alkynylene” refers to a linear saturated divalent hydrocarbon radical or a branched saturated divalent hydrocarbon radical having the number of carbon atoms indicated in the prefix and containing at least one triple bond, e.g. ethynylene, propynylene, 1-methylpropynylene, 2-methylpropynylene, butynylene, pentynylene, and the like. Preferably, an alkynylene group is a C₂₋₂₀ alkynylene group, more preferably a C₂₋₁₂ alkynylene group, yet more preferably a C₂₋₈ alkynylene group, and most preferably a C₂₋₄ alkynylene group.

As used herein, the term “cycloalkyl” refers to a cyclic saturated monovalent hydrocarbon radical of three to ten carbon atoms, e.g. cyclopropyl, cyclobutyl, cyclopentyl, or cyclohexyl, and the like.

As used herein, the term “cycloalkylene” refers to a cyclic saturated divalent hydrocarbon radical of three to ten carbon atoms, e.g. cyclopropylene, cyclobutylene, cyclopentylene, or cyclohexylene, and the like. Preferably, a cycloalkylene group is a C₃₋₁₀ cycloalkylene group, more preferably a C₃₋₈ cycloalkylene group, and most preferably a C₃₋₆ cycloalkylene group. As used herein, the term “heterocycyl” refers to a saturated or unsaturated monovalent monocyclic group of 4 to 8 ring atoms in which one or two ring atoms are heteroatoms selected from N, O, or S(O)ₙ, where n is an integer from 0 to 2, the remaining ring atoms being C. The heterocyclyl ring is optionally fused to a (one) aryl or heteroaryl ring as defined herein provided the aryl and heteroaryl rings are monocyclic. Additionally, one or two ring carbon atoms in the heterocyclyl ring can optionally be replaced by a —CO— group. More specifically the term heterocyclyl includes, but is not limited to, pyrrolidino, piperidino, homopiperidino, 2-oxopyrrolidinyl, 2-oxopiperidinyl, morpholino, piperazino, tetrahydropyranyl, thiomorpholino, and the like. When the heterocyclyl ring is unsaturated it can contain one or two ring double bonds, provided that the ring is not aromatic.

As used herein, the term “heterocyclylene” refers to a saturated or unsaturated divalent monocyclic group of 4 to 8 ring atoms in which one or two ring atoms are heteroatoms selected from N, O, or S(O)ₙ, where n is an integer from 0 to 2, the remaining ring atoms being C. The heterocyclylene ring is optionally fused to a (one) aryl or heteroaryl ring as defined herein provided the aryl and heteroaryl rings are monocyclic. Additionally, one or two ring carbon atoms in the heterocyclylene ring can optionally be replaced by a —CO— group. More specifically the term heterocyclylene includes, but is not limited to, pyrrolidinylene, piperidinylene, homopiperidinylene, 2-oxopyrrolidinylene, 2-oxopiperidinylene, morpholinylene, piperazinylene, tetrahydropyranylene, thiomorpholinylene, and the like. When the heterocyclylene ring is unsaturated it can contain one or two ring double bonds, provided that the ring is not aromatic.

As used herein, the term “aryl” refers to a monovalent monocyclic or bicyclic aromatic hydrocarbon radical of 6 to 10 ring atoms, e.g. phenyl or naphthyl, and the like.

As used herein, the term “arylene” refers to a divalent monocyclic or bicyclic aromatic hydrocarbon radical of 6 to 10 ring atoms, e.g. phenyl or naphthyl, and the like. Preferably, the arylene group is phenylene or naphthylene.

As used herein, the term “aralkyl” refers to an -(alkylene)-R radical where R is aryl as defined above. Preferably, the alkylene group is a C₁₋₂₀ alkylene group, more preferably a C₁₋₁₂ alkylene group, yet more preferably a C₁₋₈ alkylene group, and most preferably a C₁₋₄ alkylene group.

As used herein, the term “aralkylene” refers to an -(alkylene)-R divalent radical where R is arylene as defined above. Preferably, the aralkylene group is a C₇₋₂₀ aralkylene group, more preferably a C₇₋₁₄ aralkylene group, and most preferably a C₇₋₁₀ aralkylene group.

As used herein, the term “heteroaryl” refers to a monovalent monocyclic or bicyclic aromatic radical of 5 to 10 ring atoms where one or more, preferably one, two, or three, ring atoms are heteroatom selected from N, O, or S, the remaining ring atoms being carbon. Representative examples include, but are not limited to, pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, and the like.

As used herein, the term “heteroarylene” refers to a divalent monocyclic or bicyclic aromatic radical of 5 to 10 ring atoms where one or more, preferably one, two, or three, ring atoms are heteroatom selected from N, O, or S, the remaining ring atoms being carbon. Representative examples include, but are not limited to, pyrrolylene, thienylene, thiazolylene, imidazolylene, furanylene, indolylene, isoindolylene, oxazolylene, isoxazolylene, benzothiazolylene, benzoxazolylene, quinolinylene, isoquinolinylene, pyridinylene, pyrimidinylene, pyrazinylene, pyridazinylene, triazolylene, tetrazolylene, and the like.

As used herein, the term “heteroaralkyl” refers to an -(alkylene)-R radical where R is heteroaryl as defined above. Preferable alkylene groups are as defined for aralkyl groups above.

As used herein, the term “heteroaralkylene” refers to an -(alkylene)-R divalent radical where R is heteroarylene as defined above. Preferably, the heteroaralkylene group is a C₆₋₂₀ heteroaralkylene group, more preferably a C₆₋₁₄ heteroaralkylene group, and most preferably a C₆₋₁₀ heteroaralkylene group.

Optional substituents that may be present on alkyl, alkylene, alkenyl, alkenylene, alkylnyl, alkynylene, cycloalkyl, cycloalkylene, heterocyclyl, heterocyclylene, aryl, aryl ene, aralkyl, aralkylene, heteroaryl, heteroarylene, heteroaralkyl and heteroaralkylene groups include C₁₋₁₆ alkyl or C₁₋₁₆ cycloalkyl wherein one or more non-adjacent C atoms may be replaced with O, S, N, C═O and —COO—, substituted or unsubstituted C₅₋₁₄ aryl, substituted or unsubstituted C₅₋₁₄ heteroaryl, C₁₋₁₆ alkoxy, C₁₋₁₆ alkylthio, halo, cyano and aralkyl.

As used herein, the term “alkoxy” refers to an —OR radical where R is alkyl as defined above, e.g., methoxy, ethoxy, n-propoxy, iso-propoxy, n-butoxy, iso-butoxy, tert-butoxy and the like. Preferably, an alkoxy group is a C₁₋₂₀ alkoxy group, more preferably a C₁₋₁₂ alkoxy group, yet more preferably a C₁₋₈ alkoxy group, and most preferably a C₁₋₄ alkoxy group.

As used herein, the term “alkylthio” refers to an —SR radical where R is alkyl as defined above. Preferably, an alkylthio group is a C₁₋₂₀ alkylthio group, more preferably a C₁₋₁₂ alkylthio group, yet more preferably a C₁₋₈ alkylthio group, and most preferably a C₁₋₄ alkylthio group.

As used herein, the term “halo” refers to fluoro, chloro, bromo, or iodo, preferably fluoro or chloro.

As used herein, the term “keto group” refers to a carbonyl group, wherein the carbon atom of the carbonyl is also bonded to two carbon atoms.

As used herein, the term “hydrazine” refers to a group of the formula —NH—NH₂.

As used herein, the term “hydrazide” refers to a group of formulae R′(CO)—NH—NH₂ wherein R′ may be hydrogen or C₁₋₂₀ hydrocarbyl.

As used herein, the term “hydrazone” refers to a group of the formula ═N—NH—.

As used herein, the term “amine” refers to a group of the formula —NH₂, NHR or NR₂, wherein R is a C₁₋₂₀ hydrocarbyl group.

As used herein, the term “imine” refers to a group of the formula ═N—.

As used herein, the term “hydroxyl” refers to a group of the formula —OH.

As used herein, the term “ketal” refers to a group of the formula —C(OR)₂— wherein each R is C₁₋₂₀ hydrocarbyl or the two R groups together form a hydrocarbyl ring.

As used herein, the term “thiol” refers to a group of the formula —SH.

As used herein, the term “thioketal” refers to a group of the formula —C(SR)₂— wherein each R is C₁₋₂₀ hydrocarbyl or the two R groups together form a hydrocarbyl ring.

As used herein, the term “oxime” refers to a group of the formula ═N—O—.

As used herein, the term “aminoxy” or “hydroxylamine” refers to a group of the formula —O—NH₂. R—O—NH₂ refers to alkoxylamine.

As used herein, the term “Mₙ” as applied to a polymer refers to the number average molecular weight of the polymer.

As used herein, the term “M_{w}” as applied to a polymer refers to the weight average molecular weight of the polymer.

As used herein, the term “polydispersity” (also referred to as PD or Ð_{M}) refers to the ratio of the weight average molecular weight and the number average molecular weight of a polymer, i.e. Ð_{M}=M_{w}/Mₙ. It is a measure of the uniformity of a polymer sample. A low polydispersity indicates a narrow distribution of molecular mass within the polymer sample, and a high polydispersity indicates a broad distribution of molecular mass within the polymer sample.

Antibody-Drug Conjugates

The present invention relates to an antibody-drug conjugate comprising (i) an antibody or antigen-binding fragment thereof, (ii) a polymer comprising a particular repeat unit, which is covalently bound to one or more biologically active moieties, such as small molecule drugs, optionally via a linker, and (iii) a polymer-antibody linker moiety which is covalently bound to both the polymer and the antibody or antigen-binding fragment thereof. Linker groups for attaching biologically active moieties to a polymer repeat unit are well-known in the art. Advantageously the biologically active moiety is not released from the polymer until the covalent bond between the polymer and the biologically active moiety or between the linker group and the biologically active moiety is broken, e.g. hydrolysed. The location of release of the biologically active moiety and the rate of release of the biologically active moiety can therefore be controlled by selecting an antibody that directs the ADC to the site of action, and tailoring the nature of the bond between the polymer and the biologically active moiety, or between the linker group and the biologically active moiety.

The antibody-drug conjugate of the invention comprises:
(i) an antibody or antigen-binding fragment thereof;
(ii) a polymer comprising a repeat unit of Formula (I):

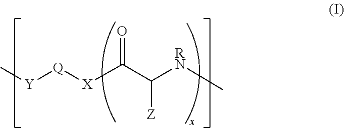

wherein:
X is selected from O, NH, NR^{A} and S;
Y is selected from C═O, C═NH, C═NR^{A} and C═S;
R is hydrogen or C₁₋₂₀ hydrocarbyl;
R^{A} is C₁₋₂₀ hydrocarbyl;
each Q is independently selected from —CH₂(NMe(C═O)CH₂)ₒ—, -T¹O(CH₂C₂O)ₛT²- and -T¹O(CH₂CH₂C₂O)ₛT²-, wherein T¹ is selected from a divalent methylene, ethylene, propylene or butylene radical, and T² is selected from a divalent methylene, ethylene, propylene or butylene radical;
o is an integer from 0 to 100;
s is an integer from 0 to 150;
x is an integer from 1 to 6; and
each Z is independently selected from a group of formula (i), (ii), (iii), (iv) or (v):

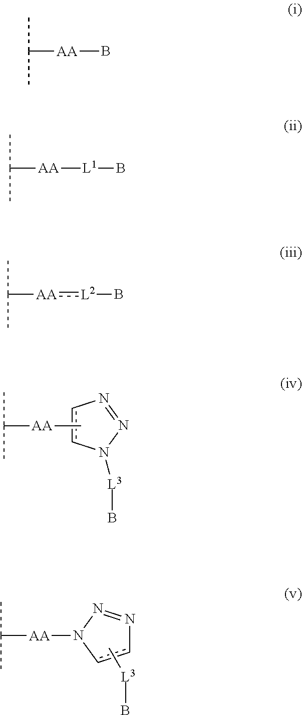

wherein,
when Z is a group of formula (i) or (ii):
   -AA- is a divalent moiety such that -AA-H represents the side chain of an amino acid;
   each L¹ is a linker group; and
   each B is a biologically active moiety;
when Z is a group of formula (iii):
   -AA= is a trivalent moiety such that -AA=O represents the side chain of an amino acid;
   each L² is a linker group;
   each dashed line represents a bond which is either present or absent; and
   each B is a biologically active moiety;
when Z is a group of formula (iv):
   -AA- is a divalent moiety such that -AA-CH═CH₂ or -AA-CCH represents the side chain of an amino acid;
   each L³ is a linker group;
   each dashed line represents a bond which is either present or absent; and
   each B is a biologically active moiety;
and when Z is a group of formula (v):
   -AA- is a divalent moiety such that -AA-N₃ represents the side chain of an amino acid;
   each L³ is a linker group;
   each dashed line represents a bond which is either present or absent; and
   each B is a biologically active moiety; and
(iii) a polymer-antibody linker which is covalently bonded to both the antibody and the polymer.

Structural Features of the Antibody

This section sets out the possible structural features of an antibody present in the antibody-drug conjugates of the invention.

The term “antibody” as referred to herein includes whole antibodies and any antigen-binding fragment (i.e., “antigen-binding portion”) or single chains thereof, as well as bispecific antibodies, and variants thereof. An antibody may also be referred to as an immunoglobulin (Ig). An antibody refers to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. Each light chain is comprised of a light chain variable region (abbreviated herein as VL) and a light chain constant region. The variable regions of the heavy and light chains contain a binding domain that interacts with an antigen. An antigen is any agent that causes the immune system of an animal body to produce an immune response, e.g. chemicals, bacteria, viruses or pollen. The VH and VL regions can be further subdivided into regions of hypervariability, termed complementarity determining regions (CDR), interspersed with regions that are more conserved, termed framework regions (FR). The constant regions of the antibodies may mediate the binding of the immunoglobulin to host tissues or factors, including various cells of the immune system (e.g., effector cells) and the first component (C1q) of the classical complement system.

The antibody may be a monoclonal antibody or a polyclonal antibody. Typically, the antibody is a monoclonal antibody. Alternatively, the antibody is a polyclonal antibody. Polyclonal antibodies are antibodies that are derived from different B cell lines. A polyclonal antibody may comprise a mixture of different immunoglobulin molecules that are directed against a specific antigen. The polyclonal antibody may comprise a mixture of different immunoglobulin molecules that bind to one or more different epitopes within an antigen molecule. Polyclonal antibodies may be produced by routine methods such as immunisation with the antigen of interest. For example a mouse or sheep capable of expressing antibodies may be immunised with an immunogenic conjugate. The animals may optionally be capable of expressing human antibody sequences. Blood may be subsequently removed and the Ig fraction purified to extract the polyclonal antibodies.

Monoclonal antibodies (mAbs) are immunoglobulin molecules that are identical to each other and have a single binding specificity and affinity for a particular epitope. Monoclonal bispecific antibodies (BsmAbs) are mAbs that can bind simultaneously to two different types of antigen. mAbs useful in the antibody-drug conjugates of the present invention can be produced by a variety of techniques, including conventional monoclonal antibody methodology, for example those disclosed in “Monoclonal Antibodies; A manual of techniques”, H Zola (CRC Press, 1988) and in “Monoclonal Hybridoma Antibodies: Techniques and Application”, SGR Hurrell (CRC Press, 1982).

The term “antigen-binding portion” of an antibody refers to a fragment of an antibody that retains the ability to specifically bind to an antigen, such as a protein, polypeptide or peptide. It has been shown that the antigen-binding function of an antibody can be performed by fragments of a full-length antibody. Examples of binding fragments encompassed within the term “antigen-binding portion” of an antibody include a Fab fragment, a F(ab′)₂ fragment, a Fab′ fragment, a Fd fragment, a Fv fragment, a dAb fragment and an isolated complementarity determining region (CDR). Single chain antibodies such as scFv and heavy chain antibodies such as VHH and camel antibodies are also intended to be encompassed within the term “antigen-binding portion” of an antibody. These antibody fragments may be obtained using conventional techniques known to those of skill in the art, and the fragments may be screened for utility in the same manner as intact antibodies.

Antibody “fragments” as defined herein may be made by truncation, e.g. by removal of one or more amino acids from its N and/or C-terminal ends. Up to 10, up to 20, up to 30, up to 40 or more amino acids may be removed from the N and/or C terminal in this way. Fragments may also be generated by one or more internal deletions. A fragment may comprise of at least 10, at least 15, at least 20, at least 25, at least 30, at least 35, at least 40, at least 45, at least 55, at least 60, at least 65, at least 70, at least 75, at least 80, at least 85, at least 90, at least 95, at least 100, at least 105, at least 120, at least 150, at least 200, at least 250, at least 300 or at least 400 consecutive amino acids from an antibody or antibody variant sequence.

Preferably, the antibody in the antibody-drug conjugate of the present invention is selected from Gemtuzumab hP67.6 humanized IgG4, Brentuximab Chimeric IgG1, Trastuzumab Humanized IgG1, Inotuzumab G5/44 Humanized IgG4, Glembatumumab Fully human IgG1, Anetumab Anti-mesothelin fully humana IgG1, Mirvetuximabb M9346A Humanized IgG1, Depatuxizumabb (ABT-806) Humanized IgG1, Rovalpituzumab (SC16) Humanized IgG1, and Vadastuximabb Humanized IgG1.

Structural Features of the Polymer

This section sets out the possible structural features of the polymer present in the antibody-drug conjugates of the invention.

The polymer of the antibody-drug conjugates of the present invention can be derived from:

(i) one or more compounds of Formula (IIa):

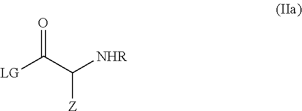

wherein LG is a leaving group under addition-elimination reaction conditions, and R and Z are as defined above for the repeat unit of Formula (I); and

(ii) a compound of Formula (IIb):

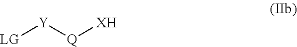

wherein LG is a leaving group under addition-elimination reaction conditions, and Q, X and Y are as defined above for the repeat unit of Formula (I).

Addition-elimination conditions are well-known to a person skilled in the art. Typically, addition-elimination conditions are any reaction conditions under which a nucleophilic (i.e. electron-rich) moiety can add to an unsaturated carbon atom to form a covalent σ-bond to that carbon atom, resulting in the disruption of a π-bond to the carbon atom, and the subsequent re-formation of said π-bond and the concomitant breaking of a σ-bond between said carbon atom and one of its other substituents, which is typically a net electron-withdrawing moiety, to eliminate that substituent.

In the polymer of the antibody-drug conjugates of the present invention, x may be 1, 2, 3, 4, 5 or 6. Preferably, however, x is 1, 2, 3, 4 or 5, still more preferably 1, 2, 3 or 4, yet more preferably 1, 2 or 3, even more preferably 1 or 2, and particularly preferably 1. Preferably, x is 1. Preferably therefore the polymer of the antibody-drug conjugates of the present invention comprises a repeat unit of Formula (Ia):

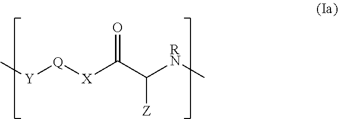

wherein Q, R, X, Y and Z are as defined above in relation to Formula (I).

The polymers are preferably derived from one or more compounds of Formula (IIa) in which R is hydrogen. More preferably, R is hydrogen in all the compounds of Formula (IIa) from which the polymer is derived.

The polymers are preferably derived from one or more compounds of Formula (IIa) and/or a compound of Formula (IIb) wherein LG is selected from Cl, OH, OR′, SH, SR′, NH₂, NHR′, NR′₂, O-2-Cl-Trt, ODmb, O-2-PhⁱPr, O-EDOTn-Ph, O—NHS, OFm, ODmab and OCam. Still more preferably LG is selected from OMe, OEt, O^{t}Bu, O-2-Cl-Trt, ODmb, O-2-PhⁱPr, O-EDOTn-Ph, O—NHS, OFm, ODmab and OCam. LG in the one or more compounds of Formula (IIa) and/or LG in Formula (IIb) may be the same or different.

As defined herein, 2-Cl-Trt refers to 2-chlorotrityl. As defined herein, Dmb refers to 2,4-dimethoxybenzyl. As defined herein, 2-PhⁱPr refers to 2-phenylisopropyl. As defined herein, Fm refers to 9-fluorenylmethyl. As defined herein, Dmab refers to 4-(N-[1-(4,4-dimethyl-2,6-dioxocyclohexylidene)-3-methylbutyl]-amino)benzyl. As defined herein, NHS refers to N-hydroxysuccinamide. As defined herein, Cam refers to carbamoylmethyl. As defined herein, aryl-EDOTn refers to a moiety having the following formula:

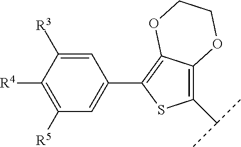

wherein R³ is H or OMe, R⁴ is H or OMe and R⁵ is H or OMe. Preferably, R³, R⁴ and R⁵ are selected such that (a) all of R³, R⁴ and R⁵ are H, (b) all of R³, R⁴ and R⁵ are OMe, (c) R³ and R⁴ are OMe and R⁵ is H, or (d) R³ and R⁴ are H and R⁵ is OMe.

When LG comprises a R′ group, R′ is preferably a C₁₋₂₀ alkyl, more preferably a C₁₋₁₂ alkyl, yet more preferably a C₁₋₈ alkyl and especially preferably a C₁₋₄ alkyl. Representative examples of suitable alkyl groups include methyl, ethyl, propyl, isopropyl, butyl, isobutyl and tert-butyl. Methyl, ethyl and tert-butyl are particularly preferred alkyl groups.

Typically, in the polymer of the antibody-drug conjugates of the present invention, Q is -T¹O(CH₂C₂O)ₛT²- or -T¹O(CH₂CH₂CH₂O)ₛT²-. In this embodiment, T¹ is preferably —CH₂—, —CH₂CH₂—, —CH₂CH₂CH₂— or —CH₂CH₂CH₂CH₂—, and is more preferably —CH₂CH₂— or —CH₂CH₂CH₂—. In this embodiment, T² is preferably —CH₂—, —CH₂CH₂—, —CH₂CH₂CH₂— or —CH₂CH₂CH₂CH₂—, and is more preferably —CH₂CH₂— or —CH₂CH₂CH₂—. T¹ and T² may be the same or different. Preferably, T¹ and T² are the same. Typically, both T¹ and T² are selected from —CH₂—, —CH₂CH₂—, —CH₂CH₂CH₂— and —CH₂CH₂CH₂CH₂—, preferably wherein both T¹ and T² are selected from —CH₂C₂— and —CH₂CH₂CH₂—, and more preferably wherein both T¹ and T² are —CH₂CH₂—.

Alternatively, in the polymer of the antibody-drug conjugates of the present invention, Q may be —CH₂(NMe(C═O)CH₂)ₒ—.

Each Q in Formula (I) may be the same or different. Preferably, each Q in Formula (I) is the same. Alternatively, each Q in Formula (I) is different.

For the avoidance of doubt, the left-hand side of the Q moiety as drawn is covalently bonded to the Y moiety in Formula (I), and the right-hand side of the Q moiety as drawn is covalently bonded to the X moiety in Formula (I).

In the polymers of the present antibody-drug conjugates, X is preferably O, NH, or NR′. Still more preferably X is O or NH. Yet more preferably, X is NH. In further preferred polymers, Y is (C═O). In a particularly preferable embodiment, X is NH and Y is (C═O).

In a further preferable embodiment, the compound of Formula (IIb) is derived from a polyethyleneglycol (PEG) or a polypropylene glycol. Preferably in this case, the compound of Formula (IIb) is derived from PEG 400, PEG 500, PEG 600, PEG 1000, PEG 1500, PEG 2000, PEG 3000, PEG 4000 and PEG 5000. Yet more preferably, X is NH, Y is C═O, Q is -T¹O(CH₂C₂O)ₛT²- or -T¹O(CH₂CH₂C₂O)ₛT²- and both T¹ and T² are —CH₂CH₂—. Most preferably, X is NH, Y is (C═O) and Q is —CH₂CH₂O(CH₂C₂O)ₛCH₂CH₂—. Preferably the compound of Formula (IIb) has a molecular weight of from 200 to 2200, and more preferably has a molecular weight of from 400 to 1200.

s is preferably an integer from 0 to 150, more preferably from 1 to 100, still more preferably from 1 to 50, yet more preferably from 3 to 35, and even more preferably from 7 to 23. Thus, in a particularly preferred embodiment, Q is —CH₂CH₂O(CH₂C₂O)ₛCH₂C₂— and s is an integer from 0 to 150, more preferably more preferably from 1 to 100, still more preferably from 1 to 50, yet more preferably from 3 to 35, and even more preferably from 7 to 23. In an even more preferred embodiment, X is NH, Y is (C═O), Q is —CH₂CH₂O(CH₂C₂O)ₛCH₂C₂— and s is an integer from 0 to 150, more preferably more preferably from 1 to 100, still more preferably from 1 to 50, yet more preferably from 3 to 35, and even more preferably from 7 to 23.

In another preferred embodiment, the compound of Formula (IIb) is derived from poly(sarcosine) or an ester thereof. In this embodiment, Q is —CH₂(NMe(C═O)CH₂)ₒ—. Yet more preferably, in this embodiment, X is NH or NR′, more preferably NR′ and still more preferably NMe. Even more preferably, Q is —CH₂(NMe(C═O)CH₂)ₒ—, X is NMe, and Y is (C═O). Still more preferably, Q is —CH₂(NMe(C═O)CH₂)ₒ—, X is NMe, Y is (C═O). Preferably the poly(sarcosine) or ester thereof has a molecular weight of from 350 to 1800. o is preferably an integer from 0 to 100, more preferably from 1 to 75, still more preferably from 2 to 50, and most preferably from 5 to 25. Thus, in a particularly preferred embodiment, Q is —CH₂(NMe(C═O)CH₂)ₒ—, X is NMe, Y is (C═O) and o is an integer from 0 to 100, more preferably from 1 to 75, still more preferably from 2 to 50, and most preferably from 5 to 25.

In the polymers of the antibody-drug conjugates, each Z is independently selected from a group of formula (i), (ii), (iii), (iv) or (v):

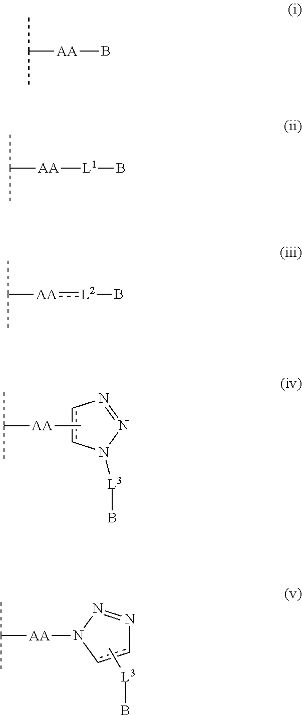

For the avoidance of doubt, the left-hand terminus of each of formulae (i) to (v) as drawn is attached to a carbon atom of the polymer backbone. Thus, in a repeat unit of Formula (I), the moiety -AA- is directly covalently bound to a carbon atom of the polymer backbone.

Thus, in one embodiment, Z is a group of formula (i). In this embodiment, there is no linker group between the amino acid side chain of the polymer and the biologically active moiety. In this embodiment, -AA- is a divalent moiety such that -AA-H represents the side chain of an amino acid. Typically, the biologically active moiety B is covalently bound to the -AA- moiety via a heteroatom on -AA-. Preferably, therefore, in this embodiment -AA-H represents the side chain of an amino acid comprising a heteroatom in its side chain. More preferably, -AA-H represents the side chain of an amino acid selected from serine, cysteine, threonine, asparagine, glutamine, aspartic acid, glutamic acid, lysine, arginine, tyrosine, tryptophan, histidine, ornithine, hydroxytryptophan, homoserine, homocysteine, allothreonine, selenocysteine, and selenohomocysteine, α-aminoglycine, diaminoacetic acid, 2,3-diaminopropionic acid and α,γ-diaminobutyric acid. In another preferable aspect of this embodiment, -AA-H is —(CH₂)ₙ—NH₂, wherein n is an integer from 0 to 10, preferably from 1 to 8, more preferably from 2 to 6, and most preferably 3 or 4. Yet more preferably, -AA-H represents the side chain of an amino acid selected from serine, cysteine, threonine, lysine and ornithine. Most preferably, -AA-H represents the side chain of lysine.

In another embodiment, Z is a group of formula (ii). In this embodiment, there is a linker group L¹ between the amino acid side chain of the polymer and the biologically active moiety. In other words, typically the antibody-drug conjugates of the present invention comprise a linker between the amino acid side chain of the polymer backbone and the biologically active moiety.

In this embodiment, -AA- is a divalent moiety such that -AA-H represents the side chain of an amino acid. Typically, the linker group I) is covalently bound to the -AA- moiety via a heteroatom on -AA-. Preferably, therefore, in this embodiment -AA-H represents the side chain of an amino acid comprising a heteroatom in its side chain. More preferably, -AA-H represents the side chain of an amino acid selected from serine, cysteine, threonine, asparagine, glutamine, aspartic acid, glutamic acid, lysine, arginine, tyrosine, tryptophan, histidine, ornithine, hydroxytryptophan, homoserine, homocysteine, allothreonine, selenocysteine, and selenohomocysteine, α-aminoglycine, diaminoacetic acid, 2,3-diaminopropionic and α,γ-diaminobutyric acid. In another preferable aspect of this embodiment, -AA-H is —(CH₂)ₙ—NH₂, wherein n is an integer from 0 to 10, preferably from 1 to 8, more preferably from 2 to 6, and most preferably 3 or 4. Yet more preferably, -AA-H represents the side chain of an amino acid selected from serine, cysteine, threonine, lysine and ornithine. Most preferably, -AA-H represents the side chain of lysine.

In this embodiment where Z is a group of formula (ii), the linker group L¹ may be any linker group suitable for connecting a biologically active moiety to the polymer backbone via covalent linkages. Such linker groups are well-known in the art. Preferably, L¹ has a molecular weight of from 14 to 4000 Da, more preferably from 28 to 2000 Da, still more preferably from 50 to 1000 Da, and yet more preferably from 100 to 500 Da. The linker group L¹ may, for example, comprise a hydrazone moiety, an oxime moiety, an imine moiety, a ketal moiety, a thioketal moiety, a carbamate moiety, a thiosemicarbozone moiety, a thiazolidine moiety, a thioester moiety, a disulfide moiety, a thioether moiety, an amide moiety or a tetrahydro-1H-pyrido[3,4-b]indole moiety. Thus, the linker group L¹ may be formed, for example, in a condensation reaction, an oxidation reaction, a Pictet-Spengler reaction, a native ligation reaction, a trapped Knoevenagel reaction, or a tandem Knoevenagel condensation-Michael addition.

The linker group L¹ is preferably a group of formula —V-L′-V²—, wherein:
V¹ is selected from

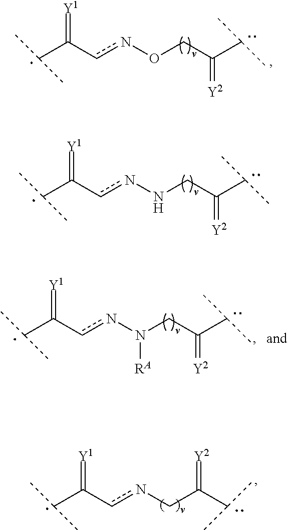

wherein
   • denotes the point of attachment to -AA-;
   •• denotes the point of attachment to -L′-;
   Y¹ is selected from O, S and NH, and is preferably O;
   Y² is selected from O, S and NH, and is preferably O;
   R^{A} is C₁₋₂₀ hydrocarbyl;
   v is an integer from 1 to 100, preferably from 1 to 50, more preferably from 1 to 20, yet more preferably from 1 to 12, still more preferably from 2 to 8, and most preferably from 2 to 6; and
   a dashed line represents an optionally present bond;
L′ is selected from a bond, C₁₋₂₀ alkylene, C₁₋₂₀ alkenylene, C₁₋₂₀ alkynylene, C₆₋₁₀ arylene (e.g. phenylene or naphthylene), C₇₋₂₀ aralkylene, C₃₋₁₀ cycloalkylene, C₄₋₈ heterocycloalkylene, C₅₋₁₀ heteroarylene, C₆₋₂₀ heteroaralkylene, —(O—K)ᵢ—, —(NH—K)ᵢ—, —(NR′—K)ᵢ—, a polyester having a molecular weight of from 116 to 2000 Da, a polyamide having a molecular weight of from 114 to 2000 Da, and a moiety —W— wherein H—W—OH is an amino acid or a peptide containing from two to twenty naturally-occurring or synthetic amino acid subunits;
V² is selected from —OV—, —NHV—, —NR^{A}V—, —SV—, —S—, —VS—, —OVS—, —NHVS—, —NR^{A}VS—, —SVS—, —V—(C═O)—, —V—O(C═O)—, —V—NH(C═O)—, —V—NR^{A}(C═O)—, —V—S(C═O)—, —V—(C═NH)—, —V—O(C═NH)—, —V—NH(C═NH)—, —V—NR^{A}(C═NH)—, —V—S(C═NH)—, —V—(C═NR^{A})—, —V—O(C═NR^{A})—, —V—NH(C═NR^{A})—, —V—NR^{A}(C═NR^{A})—, —V—S(C═NR^{A})—, —OV—(C═O)—, —OV—O(C═O)—, —OV—NH(C═O)—, —OV—NR^{A}(C═O)—, —OV—S(C═O)—, —OV—(C═NH)—, —OV—O(C═NH)—, —OV—NH(C═NH)—, —OV—NR^{A}(C═NH)—, —OV—S(C═NH)—, —OV—(C═NR^{A})—, —OV—O(C═NR^{A})—, —OV—NH(C═NR^{A})—, —OV—NR^{A}(C═NR^{A})—, —OV—S(C═NR^{A})—, —NHV—(C═O)—, —NHV—O(C═O)—, —NHV—NH(C═O)—, —NHV—NR^{A}(C═O)—, —NHV—S(C═O)—, —NHV—(C═NH)—, —NHV—O(C═NH)—, —NHV—NH(C═NH)—, —NHV—NR^{A}(C═NH)—, —NHV—S(C═NH)—, —NHV—(C═NR^{A})—, —NHV—O(C═NR^{A})—, —NHV—NH(C═NR^{A})—, —NHV—NR^{A}(C═NR^{A})—, —NHV—S(C═NR^{A})—, —NR^{A}V—(C═O)—, —NR^{A}V—O(C═O)—, —NR^{A}V—NH(C═O)—, —NR^{A}V—NR^{A}(C═O)—, —NR^{A}V—S(C═O)—, —NR^{A}V—(C═NH)—, —NR^{A}V—O(C═NH)—, —NR^{A}V—NH(C═NH)—, —NR^{A}V—NR^{A}(C═NH)—, —NR^{A}V—S(C═NH)—, —NR^{A}V—(C═NR^{A})—, —NR^{A}V—O(C═NR^{A})—, —NR^{A}V—NH(C═NR^{A})—, —NR^{A}V—NR^{A}(C═NR^{A})—, —NR^{A}V—S(C═NR^{A})—, —SV—(C═O)—, —SV—O(C═O)—, —SV—NH(C═O)—, —SV—NR^{A}(C═O)—, —SV—S(C═O)—, —SV—(C═NH)—, —SV—O(C═NH)—, —SV—NH(C═NH)—, —SV—NR^{A}(C═NH)—, —SV—S(C═NH)—, —SV—(C═NR^{A})—, —SV—O(C═NR^{A})—, —SV—NH(C═NR^{A})—, —SV—NR^{A}(C═NR^{A})—, —SV—S(C═NR^{A})—, -J-O(C═O)—, —O-J-O(C═O)—, —S-J-O(C═O)—, —NH-J-O(C═O)—, —NR^{A}-J-O(C═O)—, a polyether e.g. poly(alkylene glycol) having a molecular weight of from 76 to 2000 Da, a polyamine having a molecular weight of from 75 to 2000 Da, a polyester having a molecular weight of from 116 to 2000 Da, a polyamide having a molecular weight of from 114 to 2000 Da, and a moiety —W— wherein H—W—OH is an amino acid or a peptide containing from two to twenty naturally-occurring or synthetic amino acid subunits;
V is selected from C₁₋₂₀ alkylene, C₁₋₂₀ alkenylene, C₁₋₂₀ alkynylene, C₆₋₁₀ arylene (e.g. phenylene or naphthylene), C₇₋₂₀ aralkylene, C₃₋₁₀ cycloalkylene, C₄₋₈ heterocycloalkylene, C₅₋₁₀ heteroarylene, and C₆₋₂₀ heteroaralkylene;
J is a phenyl group which carries a sugar substituent and, para or ortho to the sugar substituent, a methylene group or a moiety —(CH═CH)ₖ—CH₂—, wherein k is an integer from 1 to 10, further wherein the methylene group or moiety —(CH═CH)ₖ13 CH₂— is directly bonded to the —O(C═O)— group proximal to the biologically active moiety B, and a carbon of the phenyl ring is directly bonded to the remainder of the linker group distal to the biologically active moiety B;
each K is the same or different and represents C₁₋₁₀ alkylene;
i is an integer from 1 to 100, preferably from 1 to 50, and more preferably from 2 to 20; and
R^{A} is C₁₋₂₀ hydrocarbyl.

Preferably, the moiety —V¹-L′-V²— terminates at the right-hand side in a nucleophilic heteroatom (such as —NH—, —O— or —S—), or in a carbonyl derivative (such as —(C═O)—, —(C═S)—, —(C═NH)— or —(C═NR^{A})—, and preferably —(C═O)—).

More preferably, the linker group L¹ is —(C═O)—C(H)═N—O—(CH₂)ᵥ—(C═O)-L′-V²—, —(C═O)—C(H)═N—NH—(CH₂)ᵥ—(C═O)-L′-V²— or —(C═O)—C(H)═N—(CH₂)ᵥ—(C═O)-L′-V², wherein L′ is as defined above and V² is selected from —V—(C═O)—, —V—O(C═O)—, —V—NH(C═O)—, —V—NR′(C═O)—, —V—S(C═O)—, —OV—(C═O)—, —OV—O(C═O)—, —OV—NH(C═O)—, —OV—NR′(C═O)—, —OV—S(C═O)—, —NHV—(C═O)—, —NHV—O(C═O)—, —NHV—NH(C═O)—, —NHV—NR′(C═O)—, —NHV—S(C═O)—, —NR′V—(C═O)—, —NR′V—O(C═O)—, —NR′V—NH(C═O)—, —NR′V—NR′(C═O)—, —NR′V—S(C═O)—, —SV—(C═O)—, —SV—O(C═O)—, —SV—NH(C═O)—, —SV—NR′(C═O)—, —SV—S(C═O)—, -J-O(C═O)—, —O-J-O(C═O)—, —S-J-O(C═O)—, —NH-J-O(C═O)—, —NR′-J-O(C═O)—, a polyester having a molecular weight of from 116 to 2000 Da, a polyamide having a molecular weight of from 114 to 2000 Da, and a moiety —W—, or, when L′ is a moiety —W—, V² may additionally be a bond. Preferably, the linker group L¹ is —(C═O)—C(H)═N—O—(CH₂)ᵥ—(C═O)-L′-V²—, —(C═O)—C(H)═N—NH—(CH₂)ᵥ—(C═O)-L′-V²— or —(C═O)—C(H)═N—(CH₂)ᵥ—(C═O)-L′-V² and the end of the linker distal to the -AA- moiety terminates in a carbonyl group.

A particularly preferred linker group L¹ is selected from —(C═O)—C(H)═N—NH—CH₂—(C═O)-Val-Cit-PAB-(C═O)—, —(C═O)—C(H)═N—O—CH₂—(C═O)-Val-Cit-PAB-(C═O)—, —(C═O)—C(H)═N—CH₂—(C═O)-Val-Cit-PAB-(C═O)—, —(C═O)—C(H)—NH—NH—CH₂—(C═O)-Val-Cit-PAB-(C═O)—, —(C═O)—C(H)—NH—O—CH₂—(C═O)-Val-Cit-PAB-(C═O)— and —(C═O)—C(H)—NH—CH₂—(C═O)-Val-Cit-PAB-(C═O)—, wherein -Val-Cit-PAB- has the following structure:

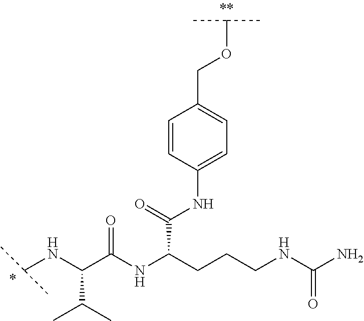

wherein * denotes the point of attachment to V¹ and ** denotes the point of attachment to —(C═O)—B.

This is a well-known linker group in the field of antibody-drug conjugates.

Most preferably, the linker group L¹ is —(C═O)—C(H)═N—O—CH₂—(C═O)-Val-Cit-PAB-(C═O)—.

Preferably, the moiety J is a phenyl group which carries a methylene group para or ortho to the sugar substituent. More preferably, the methylene group is para to the sugar substituent. Even more preferably, the sugar substituent in the moiety J is bound to the phenyl group via an oxygen atom that is also directly bonded to the anomeric carbon atom of the sugar. Yet more preferably, the sugar substituent is a six-carbon sugar. Still more preferably, the sugar substituent is selected from a sugar substituent which can be converted to a hydroxyl substituent by the action of an enzyme, such as glucuronic acid (which can be cleaved by the action of β-glucuronidase). Most preferably, the moiety J has the following structure:

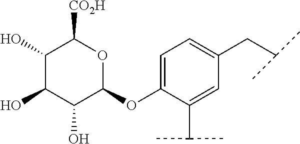

A particularly preferred linker group comprising a moiety J is selected from the following structures:

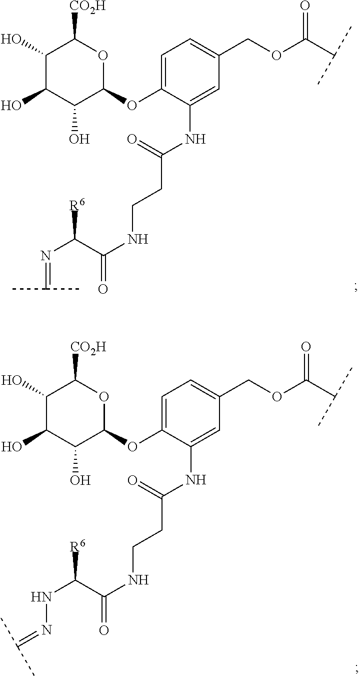

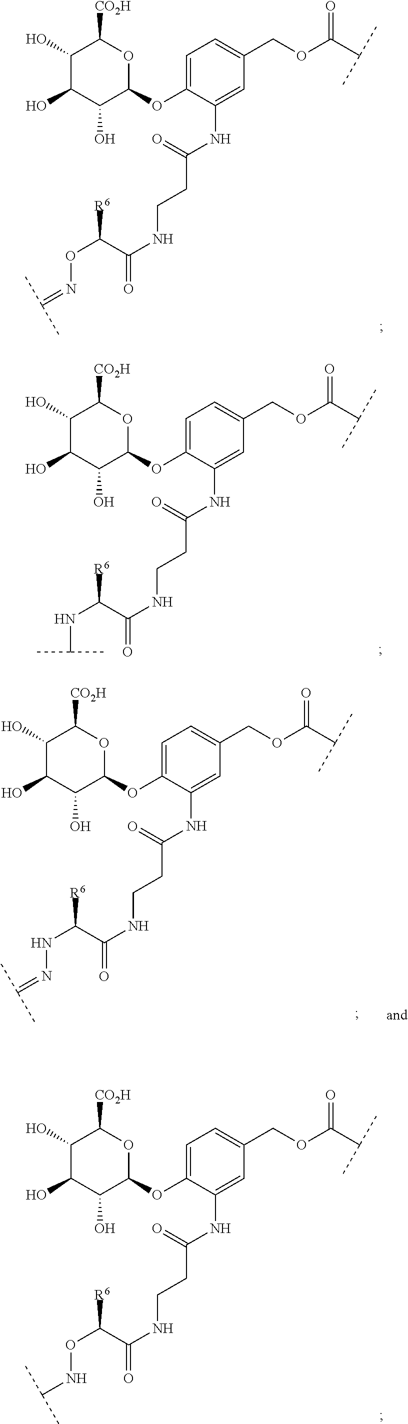

wherein R⁶ is selected from any amino acid R group or derivative thereof, e.g. H, CH₃, CH(CH₃)₂, CH₂CH(CH₃)₂, CH(CH₃)CH₂CH₃, CH₂Ph, CH₂NH₂, CH₂OH, CH₂SH, CH(OH)CH₃, CH₂CH₂SCH₃, CH₂CONH₂, CH₂CH₂CONH₂, CH₂COOH, CH₂CH₂COOH, (CH₂)₃NH(CN)NH₂, (CH₂)₄NH₂, (CH₂)₃NH₂,

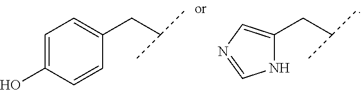

Preferably, R⁶ is selected from H, CH₃ and CH₂NH₂, and is more preferably CH₂NH₂.

Polymer-drug conjugates having a linker group L¹ selected from —(C═O)—CH₂—NH—NH—(CH₂)ᵥ—(C═O)-L′-V²—, —(C═O)—CH₂—NH—O—(CH₂)ᵥ—(C═O)-L′-V²— and —(C═O)—CH₂—NH—(CH₂)ᵥ—(C═O)-L′-V²— may be obtained by the reduction of polymer-drug conjugates having a linker group L¹ of formula —(C═O)—CH═NH—NH—(CH₂)ᵥ—(C═O)-L′-V²—, —(C═O)—CH═NH—O—(CH₂)ᵥ—(C═O)-L′-V²— and —(C═O)—CH═NH—(CH₂)ᵥ—(C═O)-L′-V²—, respectively.

In another embodiment, Z is a group of formula (iii). In this embodiment, there is a linker group L² between the amino acid side chain of the polymer and the biologically active moiety.

In this embodiment, -AA= is a trivalent moiety such that -AA=O represents the side chain of an amino acid. Typically, the linker group L² is covalently bound to the -AA- moiety via a carbon atom on -AA-. Typically, the linker group L² is covalently bound to the -AA- moiety via a double bond. Alternatively, the linker group L² is covalently bound to the -AA- moiety via a single bond. Alternatively, the linker group L² may be covalently bound to the -AA- moiety via two separate single bonds, e.g. the linker group L² may comprise a ketal or thioketal moiety. Typically, the linker group L² is covalently bound to the -AA- moiety via a double bond to a carbon atom on -AA-. Alternatively, the linker group L² is covalently bound to the -AA- moiety via a single bond to a carbon atom on -AA-. Alternatively, the linker group L² is covalently bound to the -AA- moiety via two separate single bonds to a carbon atom on -AA-.

Preferably, therefore, in this embodiment -AA=O represents the side chain of an amino acid comprising an aldehyde or a ketone in its side chain. More preferably, -AA=O represents the side chain of an amino acid selected from amino-2-keto-butyric acid, 4-acetylphenylalanine and formylglycine.

In this embodiment where Z is a group of formula (iii), the linker group L² may be any linker group suitable for connecting a biologically active moiety to the polymer backbone via covalent linkages. Such linker groups are well-known in the art. Preferably, L² has a molecular weight of from 14 to 4000 Da, more preferably from 28 to 2000 Da, still more preferably from 50 to 1000 Da, and yet more preferably from 100 to 500 Da. The linker group L² may, for example, comprise a hydrazone moiety, an oxime moiety, an imine moiety, a ketal moiety or a thioketal moiety, or a tetrahydro-1H-pyrido[3,4-b]indole moiety. Thus, the linker group L² may be formed, for example, in a condensation reaction, a Pictet-Spengler reaction, a trapped Knoevenagel reaction, or a tandem Knoevenagel condensation-Michael addition.

The linker group L² is preferably a group of formula ═V³-L′-V²—, wherein:
V³ is selected from

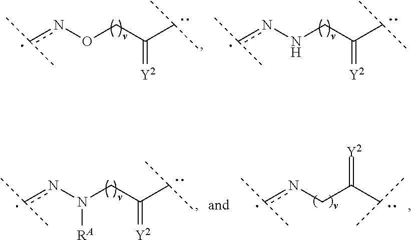

wherein •, ••, Y², R^{A} and v and a dashed line are as defined for V¹ in L¹ above;
L′ is as defined in L¹ above; and
V² is as defined in L¹ above.

Preferably, the moiety —V³-L′-V²— terminates at the right-hand side in a nucleophilic heteroatom (such as —NH—, —O— or —S—), or in a carbonyl derivative (such as —(C═O)—, —(C═S)—, —(C═NH)— or —(C═NR^{A})—, and preferably —(C═O)—).

More preferably, the linker group L² is ═N—O—(CH₂)ᵥ—(C═O)-L′-V²—, ═N—NH—(CH₂)ᵥ—(C═O)-L′-V²— or ═N—(CH₂)ᵥ—(C═O)-L′-V², wherein L′ is as defined in L¹ above and V² is selected from —V—(C═O)—, —V—O(C═O)—, —V—NH(C═O)—, —V—NR′(C═O)—, —V—S(C═O)—, —OV—(C═O)—, —OV—O(C═O)—, —OV—NH(C═O)—, —OV—NR′(C═O)—, —OV—S(C═O)—, —NHV—(C═O)—, —NHV—O(C═O)—, —NHV—NH(C═O)—, —NHV—NR′(C═O)—, —NHV—S(C═O)—, —NR′V—(C═O)—, —NR′V—O(C═O)—, —NR′V—NH(C═O)—, —NR′V—NR′(C═O)—, —NR′V—S(C═O)—, —SV—(C═O)—, —SV—O(C═O)—, —SV—NH(C═O)—, —SV—NR′(C═O)—, —SV—S(C═O)—, -J-O(C═O)—, —O-J-O(C═O)—, —S-J-O(C═O)—, —NH-J-O(C═O)—, —NR′-J-O(C═O)—, a polyester having a molecular weight of from 116 to 2000 Da, a polyamide having a molecular weight of from 114 to 2000 Da, and a moiety —W—, or, when L′ is a moiety —W—, V² may additionally be a bond. Preferably, the linker group L² is ═N—O—(CH₂)ᵥ—(C═O)-L′-V²—, ═N—NH—(CH₂)ᵥ—(C═O)-L′-V²— or ═N—(CH₂)ᵥ—(C═O)-L′-V² and the end of the linker distal to the -AA- moiety terminates in a carbonyl group.

A particularly preferred linker group L² is selected from ═N—NH—CH₂—(C═O)-Val-Cit-PAB-(C═O)—, ═N—O—CH₂—(C═O)-Val-Cit-PAB-(C═O)—, ═N—CH₂—(C═O)-Val-Cit-PAB-(C═O)—, —NH—NH—CH₂—(C═O)-Val-Cit-PAB-(C═O)—, —NH—O—CH₂—(C═O)-Val-Cit-PAB-(C═O)— and —NH—CH₂—(C═O)-Val-Cit-PAB-(C═O)—. Polymer-drug conjugates having a linker group L² selected from —NH—NH—(CH₂)ᵥ—(C═O)-L′-V²—, —NH—O—(CH₂)ᵥ—(C═O)-L′-V²— and —NH—(CH₂)ᵥ—(C═O)-L′-V²— may be obtained by the reduction of polymer-drug conjugates having a linker group L² of formula ═NH—NH—(CH₂)ᵥ—(C═O)-L′-V²—, ═NH—O—(CH₂)ᵥ—(C═O)-L′-V²— and ═NH—(CH₂)ᵥ—(C═O)-L′-V²—, respectively.

Most preferably, the linker group L² is ═N—O—CH₂—(C═O)-Val-Cit-PAB-(C═O)—.

In another embodiment, Z is a group of formula (iv). In this embodiment, there is a linker group L³ between the amino acid side chain of the polymer and the biologically active moiety.

In this embodiment, -AA- is a divalent moiety such that -AA-CH═CH₂ or -AA-CCH represents the side chain of an amino acid. Typically, the moiety -AA- and the linker group L³ are each covalently bound to adjacent atoms in the triazole ring; that is to say that L³ is bound at the 1-position of the 1,2,3-triazole and -AA- is bound at the 5-position of the 1,2,3-triazole. Alternatively, the moiety -AA- and the linker group are each covalently bound to non-adjacent atoms in the triazole ring; that is to say that L³ is bound at the 1-position of the 1,2,3-triazole and -AA- is bound at the 4-position of the 1,2,3-triazole. Typically, the optional double bond in the triazole ring is present. In this case, -AA- is a divalent moiety such that -AA-CCH represents the side chain of an amino acid. Alternatively, the optional double bond in the triazole ring is absent, i.e. the triazole ring is a 4,5-dehydro-1H-1,2,3-triazole ring. In this case, -AA- is a divalent moiety such that -AA-CH═CH₂ represents the side chain of an amino acid.

In this embodiment, -AA-CH═CH₂ represents the side chain of an amino acid comprising an alkene in its side chain, and -AA-CCH represents the side chain of an amino acid comprising an alkyne in its side chain. In this embodiment, when -AA-CH═CH₂ represents the side chain of an amino acid comprising an alkene in its side chain, the amino acid is preferably homoallylglycine. In this embodiment, when -AA-CCH represents the side chain of an amino acid comprising an alkyne in its side chain, the amino acid is preferably selected from 4-ethynylphenylalanine, 4-propargyloxyphenylalanine, propargylglycine, 4-(2-propynyl)proline, 2-amino-6-({[(1R,8S)-bicyclo[6.1.0]non-4-yn-9-ylmethoxy]carbonyl}amino)hexanoic acid and homopropargylglycine.

In this embodiment where Z is a group of formula (iv), the linker group L³ may be any linker group suitable for connecting a biologically active moiety to the polymer backbone via covalent linkages. Such linker groups are well-known in the art. Preferably, L³ has a molecular weight of from 14 to 4000 Da, more preferably from 28 to 2000 Da, still more preferably from 50 to 1000 Da, and yet more preferably from 100 to 500 Da.

The linker group L³ is preferably a group of formula —V⁴-L′-V²—, wherein:
V⁴ is —(CH₂)ᵥ—(C═Y²), wherein v and Y² are as defined for V¹ in L¹ above;
L′ is as defined in L¹ above; and
V² is as defined in L¹ above.

Preferably, the moiety —V⁴-L′-V²— terminates at the right-hand side in a nucleophilic heteroatom (such as —NH—, —O— or —S—), or in a carbonyl derivative (such as —(C═O)—, —(C═S)—, —(C═NH)— or —(C═NR^{A})—, and preferably —(C═O)—).

A particularly preferred linker group L³ is —(CH₂)ᵥ—(C═O)-Val-Cit-PAB-(C═O).

In another embodiment, Z is a group of formula (v). In this embodiment, there is a linker group L³ between the amino acid side chain of the polymer and the biologically active moiety.

In this embodiment, -AA- is a divalent moiety such that -AA-N₃ represents the side chain of an amino acid. Typically, the moiety -AA- and the linker group L³ are each covalently bound to adjacent atoms in the triazole ring; that is to say that L³ is bound at the 5-position of the 1,2,3-triazole and -AA- is bound at the 1-position of the 1,2,3-triazole. Alternatively, the moiety -AA- and the linker group are each covalently bound to non-adjacent atoms in the triazole ring; that is to say that L³ is bound at the 4-position of the 1,2,3-triazole and -AA- is bound at the 1-position of the 1,2,3-triazole. Typically, the optional double bond in the triazole ring is present. Alternatively, the optional double bond in the triazole ring is absent, i.e. the triazole ring is a 4,5-dehydro-1H-1,2,3-triazole ring.

In this embodiment, -AA-N₃ represents the side chain of an amino acid comprising an azide in its side chain, wherein the amino acid is preferably selected from 4-azidolysine, azidoornithine, azidonorleucine, azidoalanine, azidohomoalanine, 4-azidophenylalanine and 4-azidomethylphenylalanine.

In this embodiment where Z is a group of formula (v), the linker group L³ is as defined above in the case of formula (iv).

In the embodiments where Z is a group of formula (iv) or (v), the triazole ring between the -AA- and L³ moieties is typically formed in an azide-alkyne or azide-alkene cyclisation reaction.

Typically, Z is a group of formula (ii), (iii), (iv) or (v). Preferably, Z is a group of formula (ii) or (iii). Most preferably, Z is a group of formula (ii).

For the avoidance of doubt, in the above definitions of a linker group L¹ to L³, the left-hand side of the linker group as drawn attaches to the -AA- moiety, and the right-hand side of the linker group as drawn attaches to the biologically active moiety B. In the above depiction of the linker -Val-Cit-PAB-, the left-hand side shows the external bond to valine (Val) and the top shows the external bond to para-amino benzyl alcohol (PAB). In the above depiction of preferred linker groups comprising a moiety J, the bottom left shows the attachment to -AA-, and the top right shows the attachment to the biologically active moiety B.

In moiety Z, B is a biologically active moiety. A biologically active moiety is a moiety derived from a biologically active molecule (e.g. a drug) once that molecule has formed a covalent bond to either the backbone of the polymer repeat unit or, if present, a linker group. When the bond between -AA- or the linker group and B is hydrolysed, a compound B—H or B—OH is released, which is a biologically active molecule. B—OH is an example of a broader class of electrophilic biologically active molecules, designated as B-LG, where LG is any leaving group under addition-elimination reaction conditions defined herein. Thus, as used herein, a “biologically active molecule” is a said biologically active moiety which is attached to a hydrogen atom rather than to the polymer repeat unit or linker group.

Each biologically active moiety —B may be the same or different. Thus, each biologically active molecule B—H or B-LG may be the same or different. Thus. each biologically active moiety B in the antibody-drug conjugates of the present invention may be the same. However, preferably, the antibody-drug conjugate of the invention contains at least two different biologically active moieties, for example 2, 3 or 4 different biologically active moieties.

The biologically active molecule B—H or B-LG is typically independently selected from small molecule drugs, peptides, proteins, peptide mimetics, antibodies, antigens, DNA, mRNA, small interfering RNA, small hairpin RNA, microRNA, PNA, foldamers, carbohydrates, carbohydrate derivatives, non-Lipinski molecules, synthetic peptides and synthetic oligonucleotides, preferably small molecule drugs. Preferred biologically active molecules are drugs selected from anti-infective, antibiotics, antibacterial, antimicrobial, anti-inflammatory, analgesic, antihypertensive, antifungal, anti-tubercular, antiviral, anticancer, antiplatelet, antimalarial, anticonvulsant, cardio protective, antihelmintic, antiprotozoal, anti-trypanosomal, antischistosomiasis, antineoplastic, antiglaucoma, tranquilizers, hypnotics, anticonvulsants, antiparkinson, antidepressant, antihistaminic, antidiabetic, antiallurgics or proteolysis-targeting chimeras (PROTACs).

Non-limiting examples of biologically active molecules include a drug is selected from isoniazid, carbidopa, endralazine, dihydralazine, hydralazine, hydracarbazine, pheniprazine, pildralazine, octamoxin, a synthetic peptide, a synthetic oligonucleotide, a carbohydrate, a peptide mimetic, an antibody, hydrazine, Alteplase, Adalimumab, Bivalirudin, Chloroprocaine, Daptomycin, Doxazosin, Efavirenz, Hydroflumethiazide, Indapamide, Insulin Detemir, Lisinopril,peptide mimetics, Prazosin, Saxagliptin, small interfering RNA, Sulfamethylthiazole, Sulfametrole, Sulfisomidine, Tripamide, 2-p-Sulfanilylanilinoethanol, 3-Amino-4-hydroxybutyric Acid, 3-Aminopyridine-2-carboxaldehyde thiosemicarbazone (3-AP)/3-Aminopyridine-4-methyl-2-carboxaldehyde thiosemicarbazone (3-AMP/Triapine/OCX-191/OCX-0191), 4,4′-Sulfinyldianiline, 4′-(Methylsulfamoyl)sulfanilanilide, 4′-Sulfanilylsulfanilamide, 4-Amino-3-hydroxybutyric Acid, 4-Sulfanilamidosalicylic acid, 5-Hydroxytryptophan, 6-Diazo-5-oxo-L-norleucine (DON), 9-Aminoacrindine, 9-Aminocamptothecin, Abacavir, Abatacept, Acediasulfone, Acetosulfone sodium, Acyclovir, Adefovir, Alfuzosin, Amantadine, Amfenac, Amidinomycin, Amikacin, Aminolevulinic Acid, Amlodipine, Amoxicillin, Amphetamine, Amphomycin, Amphotericin B, Ampicillin, Amprenavir, Ancitabine, antibodies, antigens, Arbekacin, Aspoxicillin, Azacitidine, Azaserine, Bacampicillin, Bacitracin, BenexateHCl, Benserazide, Benzocaine, Benzylsulfamide, Bevacizumab, Bleomycins, Brodioprim, Bropirimine, Bunazosin, Butirosin, Capreomycin, carbohydrates, Carboplatin, Carubicin, Carumonam, Caspofungin, Cefaclor, Cefadroxil, Cefatrizine, Cefcapene, Cefclidin, Cefdinir, Cefditoren, Cefepime, Cefetamet, Cefinenoxime, Cefixime, Cefminox, Cefodizime, Ceforanide, Cefoselis, Cefotaxime, Cefotiam, Cefozopran, Cefpirome, Cefpodoxime, Cefprozil, Cefroxadine, Ceftazidime, Cefteram, Ceftibuten, Ceftizoxime, Ceftriaxone, Cefuzonam, Celecoxib, Cephalexin, Cephaloglycin, Cephalosporin C, Cephradine, Certolizumab, Cetoxime, Cetraxate, Cetuximab, Chlorproguanil, Cidofovir, Cilastatin, Cladribine, Clinafloxacin, Clopamide, Colesevelam, Colistin, Cyclacillin, Cycloguanil, Cyclopenthiazide, Cycloserine, Cytarabine, Dapsone, Darbepoetin Alfa, Darunavir, Daunorubicin, Decitabine, Denosumab, Dextroamphetamine, Dezocine, Dibekacin, Dideoxyadenosine, Disoproxil, DNA, Dornase Alfa, Doxorubicin, Doxycycline, Ebrotidine, Edatrexate, Eflornithine, Emtricitabine, Entecavir, Enviomycin, Epicillin, Epinastine, Epirubicin, Epoetin Alfa, Etanercept, Ethambutol, Exenatide, Famciclo Imiquimodvir, Famotidine, Filgrastim, Fingolimod, Flucytosine, Fluvoxamine, foldamers, Folic acid, Forimicins, Gabapentin, gama-Aminobutyric acid, Gemcitabine, Gemifloxacin, Gentamicin, Glatiramer Acetate, Golimumab, Histamine, Human Papilloma Quadrivalent, Hydrochlorothiazide, Idarubicin, Immune Globulin, Infliximab, Insulin Aspart, Insulin Glargine, Insulin Lispro, Interferon beta-la, Interferon beta-lb, Ipilimubab, Irsogladine, Isepamicin, Kanamycin(s), Lamivudine, Lamotrigine, Lanreotide, L-DOPA, Lenalidomide, Lenampicillin, Levodopa, Levothyroxine, Liraglutide, Lisdexamfetamine, Loracarbef, Lymecycline, Mafenide, Mantadine, Meclocycline, Melphalan, Memantine, Mesalamine, Mesalazine, Metformin, Methacycline, Methotrexate, Methyl Aminolevulinate, Methyldopa, Miboplatin, Micronomicin, microRNA, Mikamycin, Milnacipran, Minocycline, Mitoguazone, Morphazinamide, mRNA, N4-beta-D-Glucosyl sulfanilamide, Natalizumab, Natamycin, Negamycin, Neomycin, Netilmicin, Nimustine, Nolatrexed, Nomifensine, Non-Lipinski molecules, Noprysulfamide, N-Sulfanilyl-3,4-xylamide, Nystatin, Ocreotide Acetate, Omalizumab, Oseltamivir, Oxaliplatin, Palivizumab, p-Aminosalicylic acid, p-Aminosalicylic acid hydrazide, Paromomycin, Parsalmide, Pazufloxacin, Pegfilgrastim, Peginterferon alfa-2a, Pemetrexed, Penciclovir, Peplomycin, Peptide, Protein, Pexiganan, Phenyl aminosalicylate, Picloxydine, Pirarubicin, Piritrexim, Pivampicillin, Pivcefalexin, pivoxil, PNA, Polymyxin, Pralatrexate, Pregabalin, Pregabelin, Primaquine, Procaine, Proparacaine, Propoxycaine, Proxetil, p-Sulfanilylbenzylamine, Puromycin, pyrimethamine, Quinocide, Ramoplanin, Ranibizumab, Regadenoson, Remacemide, Resiquimod, Ribostamycin, Rimantadine, Ristocetin, Rituximab, Rotraxate, S-Adenosylmethionine, Salacetamide, Sampatrilat, Sevelamer, Sisomicin, Sitafloxacin, Sitagliptin, small hairpin RNA, S-Methylmethionine, Somatropin, Sparfloxacin, Streptonigrin, Succisulfone, Suclofenide, Sulfabenzamide, Sulfacetamide, Sulfachlorpyridazine, Sulfachrysoidine, Sulfacytine, Sulfadiazine, Sulfadicramide, Sulfadimethoxine, Sulfadoxine, Sulfaethidole, Sulfaguanidine, Sulfaguanole, Sulfalene, Sulfamerazine, Sulfameter, Sulfamethazine, Sulfamethizole, Sulfamethoxazole, ulfamethoxypyridazine, Sulfamidochrysoidine, Sulfamoxole, Sulfanilamide, Sulfanilic acid, Sulfanilylurea, Sulfaperine, Sulfaphenazole, Sulfaproxyline, Sulfapyrazine, Sulfasomizole, Sulfasymazine, Sulfathiazole, Sulfathiourea, Sulfatolamide, Sulfisoxazole, Sulfonamide, Sulframethomidine, Sultamicillin, Sulthiame, synthetic oligonucleotides, synthetic peptide, Tafenoquine, Talampanel, Talampicillin, Teicoplanin, Tenofovir, Terazosin, Teriparatide, Tetroxoprim, Thiamiprine, Thioguanine, Tigemonam, Tinoridine, Tirapazamine, Tobramycin, Topiramate, Tosufloxacin, Tranylcypromine, Trastuzumab, Trimazosin, Trimethoprim, Trimetrexate, Tritoqualine, Trovafloxacin, Troxacitabine, Tuberactinomycin, Tubercidin, Tyrocidine, Ustekinumab, Valacyclovir, Valdecoxib, Valganciclovir, Vancomycin, Vidarabine, Vigabatrin, Vindesine, Viomycin, Zalcitabine, Zonisamide, 2,4,6-Tribromo-m-cresol, 21-Acetoxypregnenolone, 2-p-Sulfanilylanilinoethanol, 3-Amino-4-hydroxybutyric Acid, 4-Amino-3-hydroxybutyric Acid, 4-Hexylresorcinol, 4-Sulfanilamidosalicylic acid, 5-(methylamino)-2-deoxyuridine (MADU), 5-Bromosalicylhydroxamic acid, 5-Hydroxytryptophan, 9-Aminocamptothecin, Abacavir, Abatacept, Abiraterone, Acebutolol, Acetaminophen, Acetaminosalol, Aclacinomycins, Acyclovir, Adalimumab, Ajmaline, Alclometasone, alfa-Bisabolol, all erythromycin ester derivatives, Alprenolol, Alteplase, Aluminum bis(acetylsalicylate), Amikacin, Aminochlorthenoxazin, Aminopropylon, amodiaquine, Amosulalol, Amoxicillin, Amprenavir, Ancitabine, Anidulafungin, Anileridine, Anthramycin, antibodies, antigens, Apalcillin, Apicycline, Arbekacin, Arotinolol, Artemisinin alcohol, Arzoxifene, Aspoxicillin, Atazanavir, Atenolol, Atrolactamide, Azacitidine, Azidamfenicol, Azithromycin, Bambermycins, Batimastat, Bebeerines, Beclomethasone Dipropionate, Befloxatone, Benserazide, Benzoylpas, Benzylmorphine, Betamethasone, Betaxolol, Bevacizumab, Biapenem, Bimatoprost, Bisoprolol, Bleomycins, Bosentan, Bromosalicylchloranilide, Broxuridine, Bucetin, Bucindolol, Budesonide, Bufeniode, Bufexamac, Bunitrolol, Bupranolol, Buprenorphine, Bupropion, Buramate, Buserelin, Butirosin, Butofilolol, Butorphanol, Cadralazine, Calusterone, Capecitabine, Capreomycin, Capsaicine, Carazolol, Carbidopa, carbohydrates, Carbomycin, Carteolol, Carubicin, Carvedilol, Caspofungin, CC-1065, Cefadroxil, Cefamandole, Cefatrizine, Cefbuperazone, Cefonicid, Cefoperazone, Cefoselis, Cefpiramide, Cefprozil, Celiprolol, Cephapirin sodium, Certolizumab, Cetuximab, Chloramphenicol, Chlorobutanol, Chloroxylenol, Chlorozotocin, Chlorphenesin, Chlorquinadol, Chlortetracycline Dalfopristin, Chromomycins, Cicletanine, Ciclopirox, Ciclosporine, Cidofovir, Cinchonidine, Cinchonine, Ciramadol, Cladribine, Clarithromycin, clavulanic acid, Clindamycin, Clobetasone, Clofoctol, Clomocycline, Cloxyquin, Codeine, Colesevelam, Colistin, Cyclosporin, Cytarabine, Darbepoetin Alfa, Darunavir, Dasatinib, Daunorubicin, Decitabine, Deflazacort, Delmostatin, Demeclocycline, Denosumab, Deoxydihydrostreptomycin, Desomorphine, Desonide, Desoximetasone, Desvenlafaxine, Dexamethasone, Dezocine, Diathymosulfone, Dibekacin, Didanosine, Dideoxyadenosine, Diethylstilbestrol, Diflorasone, Diflucortolone, Diflunisal, Gentisic acid, Difluprednate, Dihydroartemisithn, Dihydrocodeine, Dihydromorphine, Dihydrostreptomycin, Dihydroxyaluminum acetylsalicylate, Dilevalol, Dimepheptanol, Dirithromycin, Ditazol, DNA, Docetaxel, Dornase Alfa, Doxifluridine, Doxorubicin, Doxycycline, Droloxifene, Dromostanolone, Ecteinascidins, Edoxudine, Emtricitabine, Enocitabine, Enoxaparin, Enoxolone, Enprostil, Entacapone, Entecavir, Enviomycin, Epanolol, Epinephrine, Epirubicin, Epitiostanol, Epoetin Alfa, Eptazocine, Ertapenem, Erythromycin, Estramustine, Etanercept, Etanidazole, Ethinyl Estradiol, Ethoxazene, Ethylmorphine, Etofenamate, Etonogestrel, Etoposide, Eugenol, Everolimus, Exenatide, Ezetimibe, Fendosal, Fenoldopam Fenpentadiol, Fenretinide, Fepradinol, Fexofenadine, Filgrastim, Filipin, Flavopiridol, Flipirtine, Floctafenine, Flomoxef, Floxuridine, Fluazacort, Fluconazole, Fludrocortisone, Flumethasone, Fluocinolone, Fluocinonide, Fluocortin Butyl, Fluocortolone, Fluprednidene Acetate, Fluticasone Propionate, foldamers, Forimicins, Formestane, Formoterol, Foscarnet sodium, Fosfestrol, Fropenem, Fulvestrant, Fungichromin, Furonazide, Fusidic acid, Galantamine, Ganciclovir, Gemcitabine, Gentamicin, Glafenine, Glucametacin, Glucosulfone sodium, Glyconiazide, Golimumab. Balsalazide, Goserelin, Gramicidin(s), Guamecycline, Halcinonide, Halobetasol Propionate, Halofantrine, Halometasone, Halopredone Acetate, Human Papilloma Quadrivalent, Hydrocortisone, Hydromorphone, Hydroxypethidine, Hypericin, Ibuproxam, Idarubicin, Idoxuridine, Imipenem, Immune Globulin, Indenolol, Indinavir, Infliximab, Insulin Aspart, Insulin Detemir, Insulin Glargine, Insulin Lispro, Interferon beta-la, Interferon beta-lb, Ipilimubab, Ipratropium, Irinotecan, Isepamicin, Isoxicam, Kanamycin(s), Kethoxal, Ketobemidone, Labetalol, Lamivudine, Latanoprost, L-DOPA, Leuprolide, Levcromakalim, Levodopa, Levonorgestrel, Levorphanol, Levothyroxine, Lincomycin, Liraglutide, Lopinavir, Lornoxicam, Losartan, Loteprednol Etabonate, Lumefantrine, Lymecycline, Mannomustine, Marimastat, Mazipredone, Meclocycline, Mefloquine, Melengestrol, Meloxicam, Memetasone, Menogaril, Mepindolol, Meptazinol, Merbromin, Meropenem, Mesalamine, Mesalazine, Metazocine, Methacycline, Methyldopa, Methylprednisolone, Metipranolol, Metopon, Metoprolol, Metronidazole, Micronomicin, microRNA, Mikamycin, Miltefosine, Minocycline, Misoprostol, Mitobronitol, Mitolactol, Mitoxantrone, Mometasone Furoate, Montelukast, Mopidamol, Moprolol, Morphine, Moxalactam, mRNA, N4-beta-D-Glucosyl sulfanilamide, Nadifloxacin, Nadolol, Naftopidil, Nalbuphine, Natalizumab, Nebivolol, Negamycin, Nelfinavir, Neomycin, Netilmicin, N-Hydroxyethylpromethazine Chloride, Nifurpirinol, Nifurtoinol, Nitracrine, Nitroxoline, Nogalamycin, non-Lipinski molecules, Nordihydroguaiaretic Acid, Norlevorphanol, Normorphine, Novobiocin, Oleandomycin, Olivomycins, Olmesartan, Olsalazine, Omalizumab, Opipramol, Ornoprostil, Oryzanol A. Ganaxolone, Oxaceprol, Oxametacine, Oxycodone Pentazocine, Oxycodone, Oxymorphone, Oxyphenbutazone, Oxytetracycline, Paclitaxel and other known paclitaxel analogs, Paclitaxel, Paliperidone Palmitate, Paliperidone, Palivizumab, p-Aminosalicylic acid hydrazide, p-Aminosalicylic acid, Panipenem, Paromomycin, Pecilocin, Pegfilgrastim, Peginterferon alfa-2a, Penbutolol, Penciclovir, Pentostatin, Peplomycin, peptide mimetics, peptide, Perisoxal, Phenactropinium chloride, Phenazocine, Phenazopyridine, Phenocoll, Phenoperidine, Phentolamine, Phenyl aminosalicylate, Phenylramidol, Phenyl salicylate, Pildralazine, Pimecrolimus, Pindolol, Pipacycline, Pirarubicin, Piroxicam, p-Lactophenetide, Plaunotol, Plicamycin, PNA, Podophyllotoxin, Polymyxin, Posaconazole, Prednisolone, Prednisone, Primycin, Pristinamycin, Propranolol, protein, Protoveratrines, Puromycin, Pyrisuccideanol, Quetiapine, Ezetimibe, Quinine, Quinupristin, Raloxifene, Raltegravir, Ramoplanin, Ranibizumab, Ranimustine, Ranolazine, Ravuconazole, Rescimetol, Resiquimod, Retinoic acid (including all trans-retinioc acid), Ribavirin, Ribostamycin, Rifabutin, Rifalazil, Rifamide, Rifampicin, Rifamycin SV, Rifapentine, Rifaximin, Rimexolone, Rioprostil, Risedronic Acid, Ristocetin, Ritipenem, Ritonavir, Rituximab, Rolitetracycline, Roquinimex, Rosaprostol, Roxarsone, Roxindole, Roxithromycin, Rubijervine, Rubitecan, S-Adenosylmethionine, Salazosulfadimidine, Salicin, Tramadol, Salicylamide, Salicylanilide, Salinazid, Salmeterol, Salsalate, Sampatrilat, Sancycline, Saquinavir, Saxagliptin, Seocalcitol, Sevelamer, Siccanin, Simvastatin, Sirolimus, Sisomicin, small hairpin RNA, small interfering RNA, Somatropin, Sorivudine, Spectinomycin, Stavudine, Streptolydigin, Streptomycin, Streptonicozid, Streptozocin, Sulfasalazine, Sulfinalol, synthetic oligonucleotides, synthetic peptide, Tacrolimus, Tacrolimus. Talinolol, Teicoplanin, Telithromycin. Temoporfin, Teniposide, Tenoxicam, Tenuazonic Acid, Terfenadine, Teriparatide, Terofenamate, Tertatolol, Testosterone, Thiamphenicol, Thiostrepton, Tiazofurin, Timolol, Tiotropium, Tipranavir, Tobramycin, Tolcapone, Toloxatone, Tolterodine, Topotecan, Trans-Resveratrol [(E)-3,4′,5-trihydroxystilbene), Trastuzumab, Travoprost, Triamcinolone, Trifluridine, Trimazosin, Trimoprostil, Trospectomycin, Troxacitabine, Tuberactinomycin, Tyrocidine, Ustekinumab, Valdecoxib, Valganciclovir, Valrubicin, Vancomycin, Venlafaxine, Vidarabine, Viminol, Vinblastine, Vincristine, Vindesine, Viomycin, Virginiamycin, Voriconazole, Xanthocillin, Xibomol, Ximoprofen, Yingzhaosu A, Zalcitabine, Zanamivir, Zidovudine, Zoledronic Acid, Zolendronic Acid, Zorubicin, Zosuquidar, a peptide, protein, carbohydrate, peptide mimetic, antibody, antigen, synthetic oligonucleotide, Adalimumab, Etanercept, Pegfilgrastim, Rituximab, Bevacizumab, Insulin Glargine, Epoetin Alfa, Trastuzumab, Interferon beta-1a, Ranibizumab, Insulin Detemir, Insulin Aspart, Insulin Lispro, Filgrastim, Darbepoetin Alfa, Interferon beta-1b, Abatacept, Liraglutide, Palivizumab, Cetuximab, Ustekinumab, Denosumab, Human Papilloma Quadrivalent, Peginterferon alfa-2a, Ipilimubab, Immune Globulin, Dornase Alfa, Certolizumab, Natalizumab, Somatropin, Alteplase and Golimumab.

Particularly preferred biologically active molecules are auristatins (e.g. monomethyl auristatin E (MMAE) and MMAF), dolastatins, maytansinoids (e.g. DM1 and DM4), tubulysins, calicheamicins, duocarmycins, benzodiazepines, camptothecin, camptothecin derivatives and analogues (e.g. SN-38), amatoxin, doxorubicin, and α-amanitin.

Typically, the bond(s) between either -AA- or the linker group and B, or within the linker group, is/are acid-labile. Preferably in this case, the bond(s) is/are hydrolysed in the acidic and/or hydrolytic environment of cell compartments such as lysosome, endosome, phagosome, phagolysosome and autophagosome found in various cells such as macrophages. Preferably in this case, the bond(s) between either -AA- or the linker group and B, or at least one bond within the linker group, is/are hydrolysed in a pH of <6 and still more preferably in a pH of <5. An example of a bond hydrolysed in an acidic environment is a hydrazone bond.

Alternatively, the bond(s) between either -AA- or the linker group and B, or within the linker group, is/are labile in neutral conditions. Preferably in this case, the bond(s) between either -AA- or the linker group and B, or at least one bond within the linker group, is/are hydrolysed at a neutral pH, preferably a pH of from 6.5 to 7.5.

Alternatively, the bond(s) between either -AA- or the linker group and B, or within the linker group, is/are base-labile. Preferably the bond(s) between either -AA- or the linker group and B, or at least one bond within the linker group, is/are hydrolysed at a pH of >8 and still more preferably in a pH of >9.

The optimum pH at which the bond(s) is/are hydrolysed will depend on the precise chemical nature of the relevant bond(s).

Alternatively, the bond(s) between either -AA- or the linker group and B, or within the linker group, is/are hydrolysed in the presence of an enzyme. Preferably in this case, the bond(s) between either -AA- or the linker group and B, or at least one bond within the linker group, is/are hydrolysed by cathepsin B. An example of a bond hydrolysed enzymatically by cathepsin B is a peptide bond.

Alternatively, the bond(s) between either -AA- or the linker group and B, or within the linker group, is/are resistant to hydrolysis. For example, the bond(s) between either -AA- or the linker group and B, or at least one bond within the linker group, may be cleaved through disulfide exchange with an intracellular thiol (e.g. glutathione). An example of a bond that can be cleaved in this manner is a disulfide bond. Alternatively, the bond(s) between either -AA- or the linker group and B, or at least one bond within the linker group, may be cleaved through intracellular proteolytic degradation. An example of a bond that can be cleaved in this manner is a thioether bond.

The cleavage of the bond(s) between either -AA- or the linker group and B releases the said biologically active molecule (e.g. a drug). Preferably, there is a linker group between -AA- and the moiety B.

Typically, the biologically active molecule from which the polymer repeat unit is derived comprises a nucleophilic functional group, such as an amine, alcohol or thiol. Typically the biologically active moiety in Formula (I) is bound to -AA- or the linker group through a heteroatom in this nucleophilic functional group. In this case, the biologically active molecule has a formula B—H. Alternatively, the biologically active molecule from which the polymer repeat unit is derived may comprise an electrophilic functional group, such as a carboxylic acid, ester, thioester or α,β-unsaturated carbonyl. Typically the biologically active moiety in Formula (I) is bound to -AA- or the linker group through a carbon atom in this electrophilic functional group. In this case, the biologically active molecule has a formula B-LG, where LG is any leaving group under addition-elimination reaction conditions defined herein.

In one embodiment, the linker group L¹, L² or L³ further comprises a shielding group. Without wishing to be bound by any particular theory, such a shielding group is thought to improve the solubility of the antibody-drug conjugates of the present invention, and/or reduce agglomeration of the antibody-drug conjugates. Said shielding group is typically derived from a poly(ethylene glycol), poly(propylene glycol) or a poly(sarcosine) moiety. Thus, in a particular embodiment, Z is a group of formula (ii) wherein the group of formula (ii) is a group of formula (vi):

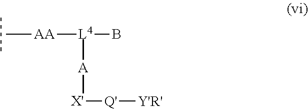

wherein:
-AA- and B are as defined in formula (ii);
each L⁴ is a linker group;
each A is independently selected from a bond, an amino acid, a peptide, a sulfonate, or a pyrophosphate diester;
each X′ is independently selected from O, NH, NR^{A′} and S;
each R′ is independently hydrogen or C₁₋₂₀ hydrocarbyl;
each R^{A′} is independently C₁₋₂₀ hydrocarbyl;
each Q′ is independently selected from —CH₂(NMe(C═O)CH₂)_{o′}—, —T′¹O(CH₂C₂O)_{s′}T′²- and -T′¹O(CH₂CH₂C₂O)_{s′}T′²-, wherein each T′¹ is independently selected from a divalent methylene, ethylene, propylene or butylene radical, and each T′² is independently selected from a divalent methylene, ethylene, propylene or butylene radical;
each o′ is independently an integer from 0 to 100;
each s′ is independently an integer from 0 to 150; and
when Q′ is -T′¹O(CH₂C₂O)_{s′}T′²- and -T′¹O(CH₂CH₂C₂O)_{s′}T′²-, each Y′ is independently selected from O, NH, NR^{A′} and S, and when Q′ is —CH₂(NMe(C═O)CH₂)_{o′}—, each Y′ is independently selected from —(C═O)—O—, —(C═O)—S—, —(C═O)—NH and —(C═O)—NR^{A′}—.

The left-hand side of the Q′ moiety as drawn is covalently bonded to the Y′ moiety in formula (vi), and the right-hand side of the Q′ moiety as drawn is covalently bonded to the X′ moiety in formula (vi).

In formula (vi), Q′ is typically -T′¹O(CH₂C₂O)ₛT′²- or -T′¹O(CH₂CH₂C₂O)ₛT′²-. Typically, is —CH₂—, —CH₂CH₂—, —CH₂CH₂CH₂— or —CH₂CH₂CH₂CH₂—, more preferably —CH₂CH₂— or —CH₂CH₂CH₂—. Typically, T′² is —CH₂—, —CH₂CH₂—, —CH₂CH₂CH₂— or —CH₂CH₂CH₂CH₂—, more preferably —CH₂CH₂— or —CH₂CH₂CH₂—. T′¹ and T′² may be the same or different. Preferably, T′¹ and T′² are the same. Typically, both T′¹ and T′² in formula (vi) are selected from —CH₂—, —CH₂CH₂—, —CH₂CH₂C₂— and —CH₂CH₂CH₂CH₂—, preferably wherein both and T′² are selected from —CH₂CH₂— and —CH₂CH₂CH₂—, and more preferably wherein both T′¹ and T′² are —CH₂CH₂—. When Q′ is -T′¹O(CH₂C₂O)ₛT′²- or -T′¹O(CH₂CH₂C₂O)ₛT′²-, X′ in formula (vi) is preferably O or NH. Yet more preferably, X′ is NH. When Q′ is -T′¹O(CH₂C₂O)ₛT′²- or -T′¹O(CH₂CH₂C₂O)ₛT′²-, Y′ in formula (vi) is preferably O or NH. Yet more preferably, Y′ is O. When Q′ is -T′¹O(CH₂C₂O)ₛT′²- or -T′¹O(CH₂CH₂C₂O)ₛT′²-, R′ in formula (vi) is preferably hydrogen, methyl or ethyl. Yet more preferably, R′ is methyl. In a particularly preferable embodiment, X′ is NH, Y′ is O and R′ is methyl.

In a further preferable embodiment, the moiety X′-Q′-Y′ in formula (vi) is derived from a polyethyleneglycol (PEG) or a polypropylene glycol. Preferably in this case, the moiety X′-Q′-Y′ is derived from PEG 400, PEG 500, PEG 600, PEG 1000, PEG 1500, PEG 2000, PEG 3000, PEG 4000 and PEG 5000. Yet more preferably, in formula (vi) X′ is NH, Y′ is O and both T′¹ and T′² are —CH₂CH₂—. Most preferably, X′ is NH, Y′ is O and Q′ is —CH₂CH₂O(CH₂C₂O)ₛCH₂CH₂—. Preferably the moiety X′-Q′-Y′ has a molecular weight of from 200 to 2200, and more preferably has a molecular weight of from 400 to 1200.

s′ is preferably an integer from 0 to 150, more preferably from 1 to 100, still more preferably from 1 to 50, yet more preferably from 3 to 35, and even more preferably from 7 to 23. Thus, in a particularly preferred embodiment, Q′ is —CH₂CH₂O(CH₂C₂O)ₛCH₂C₂— and s′ is an integer from 0 to 150, more preferably more preferably from 1 to 100, still more preferably from 1 to 50, yet more preferably from 3 to 35, and even more preferably from 7 to 23. In an even more preferred embodiment, X′ is NH, Y′ is O, Q′ is —CH₂CH₂O(CH₂C₂O)ₛCH₂CH₂— and s′ is an integer from 0 to 150, more preferably more preferably from 1 to 100, still more preferably from 1 to 50, yet more preferably from 3 to 35, and even more preferably from 7 to 23. In this embodiment, yet more preferably, R′ is methyl.

In another preferred embodiment of formula (vi), Q′ is CH₂(NMe(C═O)CH₂)ₒ—. Yet more preferably, in this embodiment, X′ is NH or NR^{A′}, more preferably NR^{A′} and still more preferably NMe. Even more preferably, Q′ is —CH₂(NMe(C═O)CH₂)ₒ—, X′ is NMe, and Y′ is —(C═O)—O—. Still more preferably, Q′ is —CH₂(NMe(C═O)CH₂)ₒ—, X′ is NMe, Y′ is —(C═O)—O— and R′ is hydrogen or methyl. In this case, the moiety X′-Q′-Y′ is derived from poly(sarcosine) or an ester thereof. Preferably the poly(sarcosine) has a molecular weight of from 350 to 1800.

o′ is preferably an integer from 0 to 100, more preferably from 1 to 75, still more preferably from 2 to 50, and most preferably from 5 to 25. Thus, in a particularly preferred embodiment, Q is —CH₂(NMe(C═O)CH₂)ₒ—, X is NMe, Y is —(C═O)—O— and o′ is an integer from 0 to 100, more preferably from 1 to 75, still more preferably from 2 to 50, and most preferably from 5 to 25. In this embodiment, yet more preferably, R′ is hydrogen or methyl.

In formula (vi), each A is independently selected from a bond, an amino acid, a peptide, a sulfonate, or a pyrophosphate diester. Preferably, A is a bond. Alternatively, A is an amino acid, a peptide, a sulfonate, a sulfonamide, or a pyrophosphate diester. When A is a sulfonate, A has the structure:

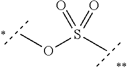

wherein * is the point of attachment to L⁴, and ** is the point of attachment to X′-Q′-Y′R′. When A is a sulfonamide, A has the structure:

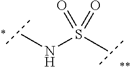

wherein * is the point of attachment to L⁴, and ** is the point of attachment to X′-Q′-Y′R′. When A is a pyrophosphate diester, A has the structure:

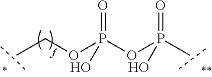

wherein * is the point of attachment to L⁴, ** is the point of attachment to X′-Q′-Y′R′, and f is an integer from 0 to 10, preferably from 1 to 6.

In formula (vi), L⁴ is typically a linker moiety of formula (x) or (xi):

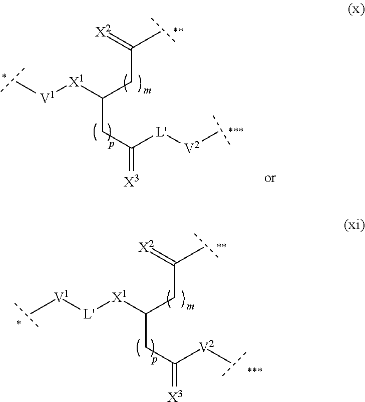

wherein:
* denotes the point of attachment to -AA-;
** denotes the point of attachment to -A-X′-Q′-Y′R′;
*** denotes the point of attachment to —B;
V¹, L′ and V² are as defined in formula (ii) above;
X¹ is selected from O, S and NH;
X² is selected from O, S and NH;
X³ is selected from O, S and NH;
R^{A} is C₁₋₂₀ hydrocarbyl;
m is an integer from 0 to 6; and
p is an integer from 0 to 6.

Thus, in formula (vi), L⁴ is typically a linker moiety of formula (x). Alternatively, L⁴ may be a linker moiety of formula (xi).

In formula (x), X¹ is preferably O or NH, more preferably NH. In formula (x), X² is preferably O. In formula (x), X³ is preferably O. More preferably, in formula (x), X¹ is NH, X² is O, and X³ is O. In formula (xi), X¹ is preferably O or NH, more preferably NH. In formula (xi), X² is preferably O. In formula (xi), X³ is preferably O. More preferably, in formula (xi), X¹ is NH, X² is O, and X³ is O.

In formula (x), preferably one of m and p is either 2 or 3, and the other is 0. In this embodiment, formula (x) is derived from aspartic acid or glutamic acid. In formula (xi), preferably one of m and p is either 2 or 3, and the other is 0. In this embodiment, formula (xi) is derived from aspartic acid or glutamic acid.

In another embodiment, Z is a group of formula (iii) wherein the group of formula (iii) is a group of formula (vii):

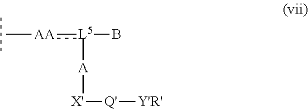

wherein:
-AA- and B are as defined in formula (iii);
each L⁵ is a linker group;
each A, X′, Y′, R′, R^{A′} and Q′ are as defined (including preferable embodiments) in formula (vi); and
each dashed line represents a bond which is either present or absent.

In formula (vii), L⁵ is typically a linker moiety of formula (xii) or (xiii):

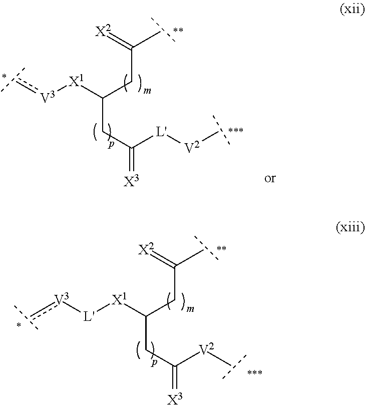

wherein *, **, ***, L′, V², X¹, X², X³ R^{A}, m and p are as defined in formula (x) or formula (xi), V³ is as defined in formula (iii), and each dashed line is a bond which is either present or absent.

Thus, in formula (vii), L⁵ is typically a linker moiety of formula (xii). Alternatively, L⁵ may be a linker moiety of formula (xiii).

In another embodiment, Z is a group of formula (iv) wherein the group of formula (iv) is a group of formula (viii):

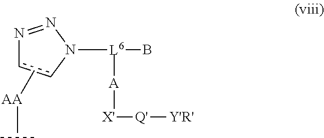

wherein:
-AA- and B are as defined in formula (iv);
each L⁶ is a linker group;
each A, X′, Y′, R′, R^{A′} and Q′ are as defined (including preferable embodiments) in formula (vi); and
each dashed line represents a bond which is either present or absent.

In formula (vii), L⁶ is typically a linker moiety of formula (xiv) or (xv):

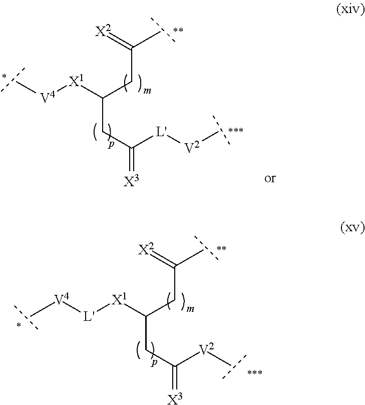

wherein *, **, ***, L′, V², X¹, X², X³ R^{A}, m and p are as defined in formula (x) or formula (xi), and V⁴ is as defined in formula (iv).

Thus, in formula (viii), L⁶ is typically a linker moiety of formula (xiv). Alternatively, L⁶ may be a linker moiety of formula (xv).

In another embodiment, Z is a group of formula (v) wherein the group of formula (v) is a group of formula (ix):

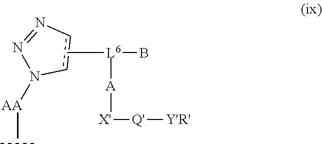

wherein:
-AA- and B are as defined in formula (v);
each L⁶ is a linker group as defined in formula (viii);
each A, X′, Y′, R′, R^{A′} and Q′ are as defined (including preferable embodiments) in formula (vi); and
each dashed line represents a bond which is either present or absent.

Structure of Polymer-Antibody Linker Moieties

This section sets out the possible structural features of the linker moiety present in the antibody-drug conjugates of the invention.

The linker moiety in the antibody-drug conjugates of the present invention may derive from any suitable compound which has at least two separate reactive functional groups: one functional group which reacts with the polymer to form a covalent bond, and a further functional group which reacts with the antibody to form a covalent bond. The antibody-drug linker moiety may be the same or different to any linker group used to attach the polymer backbone to the biologically active moiety (when such a linker group is present). Preferably, the antibody-drug linker moiety is different to the linker group used to attach the polymer backbone to the biologically active moiety.

Typically, the polymer-antibody linker is covalently bound to the polymer through the carbon atom of the —Y— moiety in the repeat unit of Formula (I), or the —NR— group in the amino acid-derived portion of the repeat unit of Formula (I). Typically, the polymer-antibody linker is covalently bound to the polymer at one of the polymer termini.

Typically, the polymer-antibody linker is covalently bound to the antibody through a reactive amino acid side chain of the antibody, e.g. the thiol group of a cysteine residue, the amino group of a lysine residue, the carboxylic acid group of a glutamic acid residue or an aspartic acid residue, the selenol group of a selenocysteine residue, or through the N-terminus of the backbone of one of the polypeptides in the antibody, or through a hydroxyl group of an oligosaccharide present in the fragment crystallisable (Fc) region of the antibody, or through aldehyde or hydroxylamine groups of glycans or non-natural residues, or through alkyne or azide groups of glycans or non-natural residues.

The polymer and the antibody may independently be covalently bound to the same atom of the linker moiety or they may be independently covalently bound to different atoms of the linker moiety. Preferably, the polymer and the antibody are independently covalently bound to different atoms of the linker moiety.

Suitable linker moieties for use in antibody-drug conjugates of the present invention include, but are not limited to, linkers derived from thiols, maleimide, monobromomaleimide, maleimide analogues, vinyl sulfones, bis(sulfone)s (e.g. Thiobridge®), allenamides, vinyl-pyridines, divinylpyrimidine, dehydroalanine, alkenes, perfluoroaromatic molecules, sulfone reagents that are Julia-Kocienski like, N-hydroxysuccinamide-ester activated carboxylate species, aldehydes, ketones, hydroxylamines, alkynes and azides.

Thus, reaction of thiols, maleimide, monobromomaleimide, maleimide analogues, vinyl sulfones, bis(sulfone)s (e.g. Thiobridge®), allenamides, vinyl-pyridines, divinylpyridine, dehydroalanine, alkenes, perfluoroaromatic species, sulfone reagents that are Julia-Kocienski like, N-hydroxysuccinamide-ester activated carboxylate species, aldehydes, ketones, hydroxylamines, alkynes and azides with both (a) the polymer backbone and (b) the antibody results in a suitable linker group. Bis(sulfones) act in this context as (bis-alkylating) reagents. Linkers can be derived from alkenes by e.g. a light-initiated thiol-ene reaction. Thus, a thiol group on an antibody can react with alkene functionality to generate a covalent link. Reaction with dehydroalanine may occur e.g. by Michael addition-elimination with a thiol group on an antibody. N-hydroxysuccinamide-ester activated carboxylate species may react with lysine groups in an antibody. Ketones, aldehydes and/or hydroxylamines may be conjugated to a glycan-modified antibody or non-natural residue via oxime bond formation or by hydrazino-Pictet-Spengler (HIPS) ligation. Alkynes and azides may be conjugated to a glycan-modified antibody or non-natural residue via click chemistry (azide-alkyne cycloaddition).

Structure of Antibody-Drug Conjugates

Most preferably, the antibody-drug conjugate of the present invention has Formula (III) or (IV):

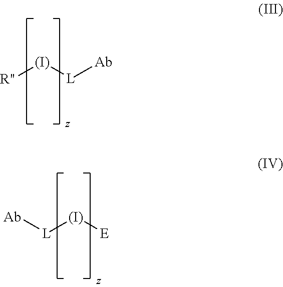

wherein:
(I) is a repeat unit of the Formula (I), as defined in any of the previous claims;
Ab is an antibody or antigen-binding fragment thereof;
L is a polymer-antibody linker as defined above;
R″ is selected from OH, OR^{A}, SH, SR^{A}, NH₂, NHR^{A} and NR^{A}₂;
E is selected from H and R^{A};
R^{A} is as defined in Formula (I); and
z is an integer from 1 to 50.

Thus, typically, the antibody-drug conjugate of the present invention has Formula (IIIa) or Formula (IVa):

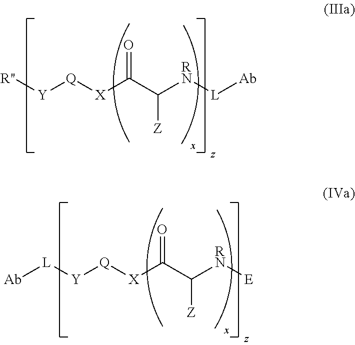

Preferably, z is an integer from 1 to 30, more preferably from 2 to 20, even more preferably from 2 to 15, and most preferably from 2 to 12.

The polymer in an antibody-drug conjugate of the present invention typically has a weight average molecular weight of 500 to 500 000 Da, more preferably 1000 to 200 000 Da, and still more preferably 1500 to 36 000 Da. Preferably, the polymer has a number average molecular weight of 500 to 500 000 Da, more preferably 1000 to 200 000 Da, still more preferably 1500 to 25 000 Da and yet more preferably 2000 to 20 000 Da. Preferably, the polymer has a polydispersity of 1 to 5, more preferably 1.05 to 4.8, still more preferably 1.1 to 2.4 and yet more preferably 1.1 to 1.5. Alternatively, the polymer has a polydispersity of from 0.9 to 1.1, preferably from 0.95 to 1.05, and most preferably about 1, i.e. preferably, the polymer is monodisperse.

The biologically active moiety present in the antibody-drug conjugates of the present invention preferably has a molecular weight of 32 to 100 000 Da. The biologically active moiety may be a small molecule drug which may be a small organic molecule, i.e. non-polymeric, or polymeric. Preferably the antibody-drug conjugate of the present invention comprises 0.5 to 90 wt %, more preferably 0.75 to 70 wt %, still more preferably 1 to 60 wt %, yet more preferably 1.5 to 50 wt %, still more preferably 1.75 to 25 wt %, and most preferably 2 to 10 wt % biologically active moiety, based on the weight of the dry antibody-drug conjugate. A key advantage of the antibody-drug conjugates of the present invention is that relatively high amounts of biologically active molecule can be incorporated into the polymer. Further, multiple polymers may bind to a single antibody. These factors, in turn, mean that high biologically active molecule loadings may be achieved. Typically, the drug-to-antibody ratio (DAR) is 4:1 or greater, preferably 5:1 or greater, more preferably 8:1 or greater, yet more preferably 10:1 or greater, still more preferably 12:1 or greater, even more preferably 15:1 or greater, and most preferably 16:1 or greater, for example 20:1 or greater.

Typically, the antibody-drug conjugates of the present invention have a solubility in water of at least 10 mg/mL, preferably at least 30 mg/mL, more preferably at least 50 mg/mL, still more preferably at least 75 mg/mL, and most preferably at least 100 mg/mL.

The present invention also provides an antibody-drug conjugate as described herein, wherein release of the biologically active moiety from the polymer is pH sensitive and is dependent upon the nature of the bond between said biologically active moiety and the repeat unit of the polymer or the linker group to which it is covalently bound.

Alternatively, the antibody may be replaced by an alternative form of targeting agent. Thus, the present invention also provides a targeting agent-drug conjugate comprising:
(i) a targeting agent;
(ii) a polymer comprising a repeat unit of Formula (I):

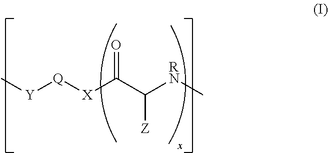

wherein:
X is selected from O, NH, NR^{A} and S;
Y is selected from C═O, C═NH, C═NR^{A} and C═S;
R is hydrogen or C₁₋₂₀ hydrocarbyl;
R^{A} is C₁₋₂₀ hydrocarbyl;
each Q is independently selected from —CH₂(NMe(C═O)CH₂)ₒ—, -T¹O(CH₂C₂O)ₛT²- and -T¹O(CH₂CH₂C₂O)ₛT²-, wherein T¹ is selected from a divalent methylene, ethylene, propylene or butylene radical, and T² is selected from a divalent methylene, ethylene, propylene or butylene radical;
o is an integer from 0 to 100;
s is an integer from 0 to 150;
x is an integer from 1 to 6; and
each Z is independently selected from a group of formula (i), (ii), (iii), (iv) or (v):

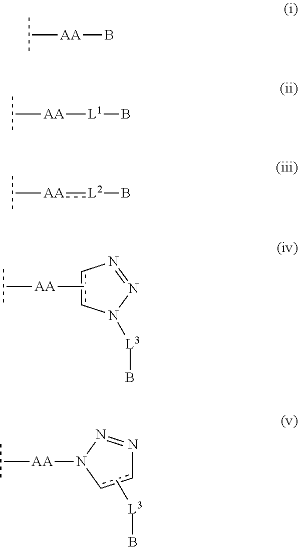

wherein,
when Z is a group of formula (i) or (ii):
   -AA- is a divalent moiety such that -AA-H represents the side chain of an amino acid;
   each L¹ is a linker group; and
   each B is a biologically active moiety;
when Z is a group of formula (iii):
   -AA= is a trivalent moiety such that -AA=O represents the side chain of an amino acid;
   each L² is a linker group;
   each dashed line represents a bond which is either present or absent; and
   each B is a biologically active moiety;
when Z is a group of formula (iv):
   -AA- is a divalent moiety such that -AA-CH═CH₂ or -AA-CCH represents the side chain of an amino acid;
   each L³ is a linker group;
   each dashed line represents a bond which is either present or absent; and
   each B is a biologically active moiety; and
when Z is a group of formula (v):
   -AA- is a divalent moiety such that -AA-N₃ represents the side chain of an amino acid;
   each L³ is a linker group;
   each dashed line represents a bond which is either present or absent; and
   each B is a biologically active moiety; and
(iii) a polymer-targeting agent linker which is covalently bonded to both the targeting agent and the polymer.

Preferable embodiments of Formula (I) are as for the antibody-drug conjugates described above.

The targeting agent is covalently bound to the polymer. Suitable targeting agents include biomolecules such as peptides, proteins, peptide mimetics, antibodies, antigens, DNA, mRNA, small interfering RNA, small hairpin RNA, microRNA, PNA, foldamers, carbohydrates, carbohydrate derivatives, non-Lipinski molecules, synthetic peptides and synthetic oligonucleotides.

The polymer-targeting agent linker may assume any of the same structures as the polymer-antibody linker that is defined above.

Most preferably, the targeting agent-drug conjugate of the present invention has Formula (V) or (VI):

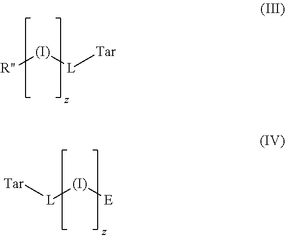

wherein:
(I) is a repeat unit of the Formula (I), as defined in any of the previous claims;
Tar is a targeting agent as defined above;
L is a polymer-antibody linker as defined above;
R″ is selected from OH, OR^{A}, SH, SR^{A}, NH₂, NHR^{A} and NR^{A}₂;
E is selected from H and R^{A};
R^{A} is as defined in Formula (I); and
z is an integer from 1 to 50.

Thus, typically, the antibody-drug conjugate of the present invention has Formula (Va) or Formula (VIa):

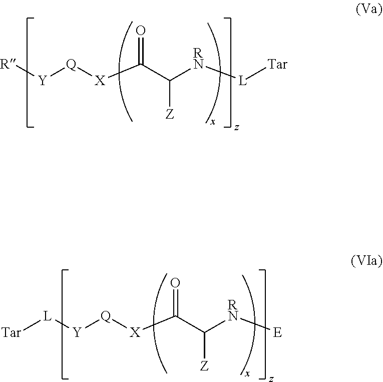

Preferably, z is an integer from 1 to 30, more preferably from 2 to 20, even more preferably from 2 to 15, and most preferably from 2 to 12. The polymer in a targeting agent-drug conjugate of the present invention typically has a weight average molecular weight of 500 to 500 000 Da, more preferably 1000 to 200 000 Da, and still more preferably 1500 to 36 000 Da. Preferably, the polymer has a number average molecular weight of 500 to 500 000 Da, more preferably 1000 to 200 000 Da, still more preferably 1500 to 25 000 Da and yet more preferably 2000 to 20 000 Da. Preferably, the polymer has a polydispersity of 1 to 5, more preferably 1.05 to 4.8, still more preferably 1.1 to 2.4 and yet more preferably 1.1 to 1.5.

The biologically active moiety present in the targeting agent-drug conjugates of the present invention preferably has a molecular weight of 32 to 100 000 Da. The biologically active moiety may be a small molecule drug which may be a small organic molecule, i.e. non-polymeric, or polymeric. Preferably the targeting agent-drug conjugate of the present invention comprises 0.5 to 90 wt %, more preferably 0.75 to 70 wt %, still more preferably 1 to 60 wt %, yet more preferably 1.5 to 50 wt %, even more preferably 1.75 to 25 wt %, and most preferably 2 to 10 wt % biologically active moiety, based on the weight of the dry antibody-drug conjugate. A key advantage of the targeting agent-drug conjugates of the present invention is that relatively high amounts of biologically active molecule can be incorporated into the polymer. Further, multiple polymers may bind to a single targeting agent. These factors, in turn, mean that high biologically active molecule loadings may be achieved. Typically, the drug-to-targeting agent ratio is 4:1 or greater, preferably 5:1 or greater, more preferably 8:1 or greater, yet more preferably 10:1 or greater, still more preferably 12:1 or greater, even more preferably 15:1 or greater, and most preferably 16:1 or greater, for example 20:1 or greater.

Each biologically active moiety B in the targeting agent-drug conjugates of the present invention may be the same. Alternatively, the targeting agent-drug conjugate of the invention contains at least two different biologically active moieties, for example 2, 3 or 4 different biologically active moieties. Preferred biologically active moieties present in the targeting-drug conjugates of the present invention are as described above in relation to antibody-drug conjugates.

Typically, the targeting agent-drug conjugates of the present invention have a solubility in water of at least 30 mg/mL, preferably at least 50 mg/mL, more preferably at least 75 mg/mL, and most preferably at least 100 mg/mL.

Methods for Manufacture of Antibody-Drug Conjugates

The present invention also relates to a method of producing an antibody-drug conjugate according to the invention.

In the below methods, each leaving group LG is preferably selected from from Cl, OH, OR′, SH, SR′, NH₂, NHR′, NR′₂, O-2-Cl-Trt, ODmb, O-2-Ph¹Pr, O-EDOTn-Ph, O—NHS, OFm, ODmab and OCam. Still more preferably LG is selected from OMe, OEt, O^{t}Bu, O-2-Cl-Trt, ODmb, O-2-Ph¹Pr, O-EDOTn-Ph, O—NHS, OFm, ODmab and OCam. LG in the one or more compounds of Formula (IIa) and/or Formula (lIIb) and/or Formula (IIc) and/or Formula (IId) and/or Formula (IIf) and/or Formula (IIg) and/or Formula (IIh) and/or Formula (IIj) and/or B-LG may be the same or different.

Typically, such a method comprises the steps of:
(a) reacting a compound of Formula (IIa):

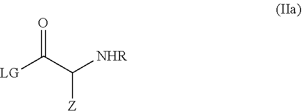

with a compound of Formula (IIb):

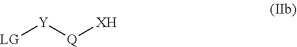

wherein Q, R, X, Y, Z and LG are as defined above;
(b) reacting the product of step (a) with a polymer-antibody linker; and
(c) reacting the product of step (b) with an antibody or antigen-binding fragment thereof.

Alternatively, the method comprises the steps of:
(a) reacting an antibody or antigen-binding fragment thereof with a polymer-antibody linker;
(b) separately, reacting a compound of Formula (IIa):

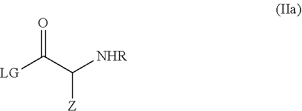

with a compound of Formula (IIb):

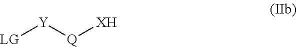

wherein Q, R, X, Y, Z and LG are as defined above; and
(c) reacting the product of step (a) with the product of step (b).

Alternatively, Z is a group of formula (i), and the method comprises the steps of:
(a) reacting a compound of Formula (IIc):

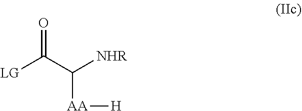

with a compound of Formula (IIb):

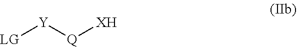

wherein Q, R, X, Y, AA and LG are as defined above;
(b) reacting the product of step (a) with a polymer-antibody linker;
(c) reacting the product of step (b) with a biologically active molecule B-LG; and
(d) reacting the product of step (c) with an antibody or antigen-binding fragment thereof.

Alternatively, Z is a group of formula (i), and the method comprises the steps of:
(a) reacting a compound of Formula (IIc):

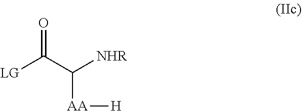

with a compound of Formula (IIb):

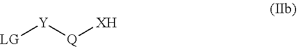

wherein Q, R, X, Y, AA and LG are as defined above;
(b) reacting the product of step (a) with a biologically active molecule B-LG;
(c) reacting the product of step (b) with a polymer-antibody linker; and
(d) reacting the product of step (c) with an antibody or antigen-binding fragment thereof.

Alternatively, Z is a group of formula (i), and the method comprises the steps of:
(a) reacting a compound of Formula (IIc):

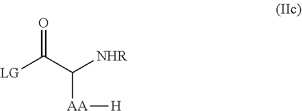

with a compound of Formula (IIb):

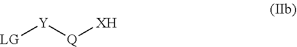

and a biologically active molecule B-LG,
wherein Q, R, X, Y, AA and LG are as defined above;
(b) reacting the product of step (a) with a polymer-antibody linker; and
(c) reacting the product of step (b) with an antibody or antigen-binding fragment thereof.

Alternatively, Z is a group of formula (i), and the method comprises the steps of:
(a) reacting an antibody or antigen-binding fragment thereof with a polymer-antibody linker;
(b) separately, reacting a compound of Formula (IIc):

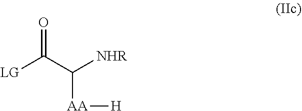

with a compound of Formula (IIb):

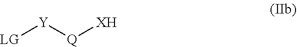

wherein Q, R, X, Y, AA and LG are as defined above;
(c) reacting the product of step (a) with the product of step (b); and
(d) reacting the product of step (c) with a biologically active molecule B-LG.

Alternatively, Z is a group of formula (i), and the method comprises the steps of:
(a) reacting an antibody or antigen-binding fragment thereof with a polymer-antibody linker;
(b) separately, reacting a compound of Formula (IIc):

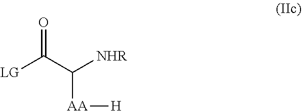

with a compound of Formula (IIb):

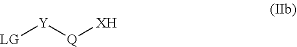

and a biologically active molecule B-LG,
wherein Q, R, X, Y, AA and LG are as defined above; and
(c) reacting the product of step (a) with the product of step (b).

Alternatively, Z is a group of formula (ii), and the method comprises the steps of:
(a) reacting a compound of Formula (IId):

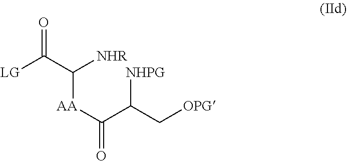

with a compound of Formula (IIb):

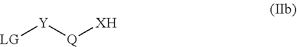

wherein Q, R, X, Y, AA and LG are as defined above, and PG and PG′ are each independently a protecting group;
(b) reacting the product of step (a) with a polymer-antibody linker;
(c) removing the protecting groups PG and PG′ under suitable reaction conditions;
(d) performing an oxidative cleavage to provide a 1,2-dicarbonyl species comprising the repeat unit Formula (IIe):

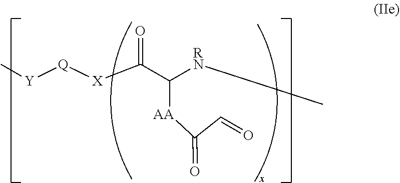

wherein x is as defined above;
(e) reacting the product of step (d) with a linker moiety H-L²-LG, wherein L² and LG are as defined above;
(f) reacting the product of step (d) with a biologically active moiety B—H; and
(g) reacting the product of step (f) with an antibody or antigen-binding fragment thereof.

Alternatively, Z is a group of formula (ii), and the method comprises the steps of:
(a) reacting a compound of Formula (IId):

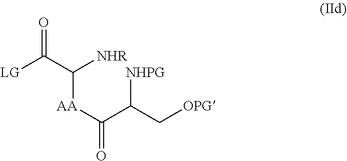

with a compound of Formula (IIb):

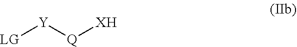

wherein Q, R, X, Y, AA and LG are as defined above, and PG and PG′ are each independently a protecting group;
(b) removing the protecting groups PG and PG′ under suitable reaction conditions;
(c) reacting the product of step (b) with a polymer-antibody linker;
(d) performing an oxidative cleavage to provide a 1,2-dicarbonyl species comprising the repeat unit Formula (IIe):

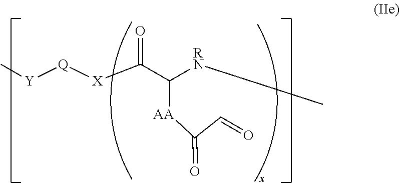

wherein x is as defined above;
(e) reacting the product of step (d) with a linker moiety H-L²-LG, wherein L² and LG are as defined above;
(f) reacting the product of step (d) with a biologically active moiety B—H; and
(g) reacting the product of step (f) with an antibody or antigen-binding fragment thereof.

Alternatively, Z is a group of formula (ii), and the method comprises the steps of:
(a) reacting a compound of Formula (IId):

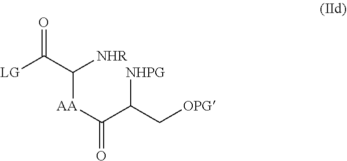

with a compound of Formula (IIb):

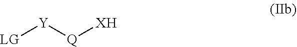

wherein Q, R, X, Y, AA and LG are as defined above, and PG and PG′ are each independently a protecting group;
(b) removing the protecting groups PG and PG′ under suitable reaction conditions;
(c) performing an oxidative cleavage to provide a 1,2-dicarbonyl species comprising the repeat unit Formula (IIe):

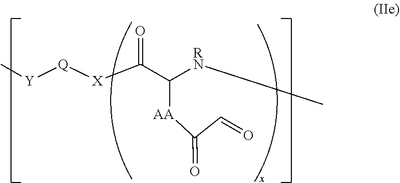

wherein x is as defined above;
(d) reacting the product of step (c) polymer-antibody linker;
(e) reacting the product of step (d) with a linker moiety H-L²-LG, wherein L² and LG are as defined above;
(f) reacting the product of step (d) with a biologically active moiety B—H; and
(g) reacting the product of step (f) with an antibody or antigen-binding fragment thereof.

Alternatively, Z is a group of formula (ii), and the method comprises the steps of:
(a) reacting a compound of Formula (IId):

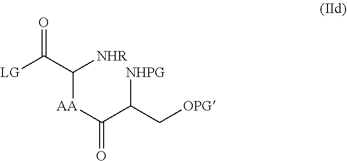

with a compound of Formula (IIb):

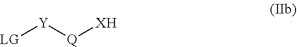

wherein Q, R, X, Y, AA and LG are as defined above, and PG and PG′ are each independently a protecting group;
(b) reacting the product of step (a) with a polymer-antibody linker;
(c) removing the protecting groups PG and PG′ under suitable reaction conditions;
(d) performing an oxidative cleavage to provide a 1,2-dicarbonyl species comprising the repeat unit Formula (IIe):

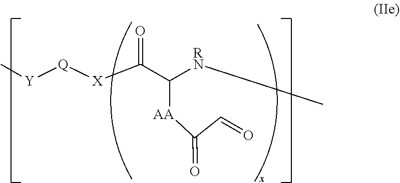

wherein x is as defined above;
(e) separately, reacting a linker moiety H-L²-LG, wherein L² and LG are as defined above, with a biologically active moiety B—H;
(f) reacting the product of step (d) with the product of step (e); and
(g) reacting the product of step (f) with an antibody or antigen-binding fragment thereof.

Alternatively, Z is a group of formula (ii), and the method comprises the steps of:
(a) reacting a compound of Formula (IId):

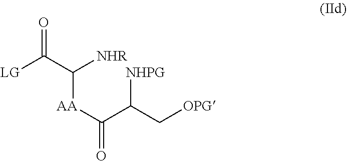

with a compound of Formula (IIb):

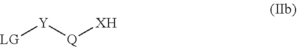

wherein Q, R, X, Y, AA and LG are as defined above, and PG and PG′ are each independently a protecting group;
(b) removing the protecting groups PG and PG′ under suitable reaction conditions;
(c) reacting the product of step (b) with a polymer-antibody linker;
(d) performing an oxidative cleavage to provide a 1,2-dicarbonyl species comprising the repeat unit Formula (IIe):

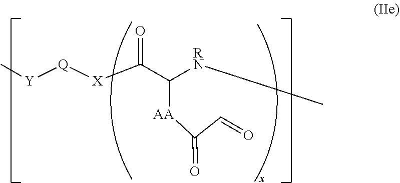

wherein x is as defined above;
(e) separately, reacting a linker moiety H-L²-LG, wherein L² and LG are as defined above, with a biologically active moiety B—H;
(f) reacting the product of step (d) with the product of step (e); and
(g) reacting the product of step (f) with an antibody or antigen-binding fragment thereof.

Alternatively, Z is a group of formula (ii), and the method comprises the steps of:
(a) reacting a compound of Formula (IId):

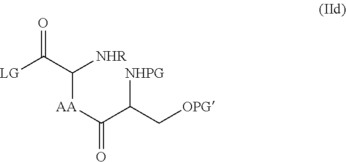

with a compound of Formula (IIb):

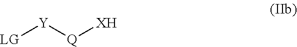

wherein Q, R, X, Y, AA and LG are as defined above, and PG and PG′ are each independently a protecting group;
(b) removing the protecting groups PG and PG′ under suitable reaction conditions;
(c) performing an oxidative cleavage to provide a 1,2-dicarbonyl species comprising the repeat unit Formula (IIe):

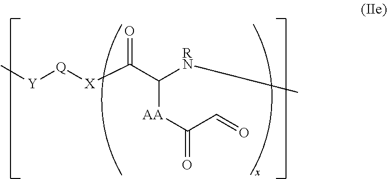

wherein x is as defined above;
(d) reacting the product of step (c) with a polymer-antibody linker;
(e) separately, reacting a linker moiety H-L²-LG, wherein L² and LG are as defined above, with a biologically active moiety B—H;
(f) reacting the product of step (d) with the product of step (e); and
(g) reacting the product of step (f) with an antibody or antigen-binding fragment thereof.

Alternatively, Z is a group of formula (ii), and the method comprises the steps of:
(a) reacting a compound of Formula (IId):

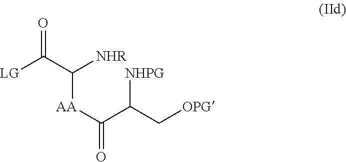

with a compound of Formula (IIb):

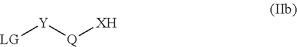

wherein Q, R, X, Y, AA and LG are as defined above, and PG and PG′ are each independently a protecting group;
(b) removing the protecting groups PG and PG′ under suitable reaction conditions;
(c) performing an oxidative cleavage to provide a 1,2-dicarbonyl species comprising the repeat unit Formula (IIe):

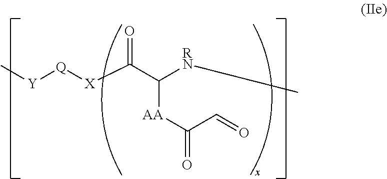

wherein x is as defined above;
(d) separately, reacting a linker moiety H-L²-LG, wherein L² and LG are as defined above, with a biologically active moiety B—H;
(e) reacting the product of step (c) with the product of step (d);
(f) reacting the product of step (e) with a polymer-antibody linker;
(g) reacting the product of step (f) with an antibody or antigen-binding fragment thereof.

Alternatively, Z is a group of formula (ii), and the method comprises the steps of:
(a) reacting an antibody or antigen-binding fragment thereof with a polymer-antibody linker;
(b) separately, reacting a compound of Formula (IId):

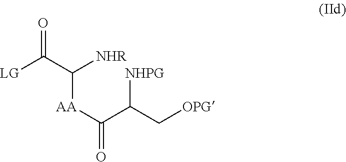

with a compound of Formula (IIb):

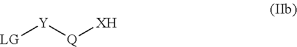

wherein Q, R, X, Y, AA and LG are as defined above, and PG and PG′ are each independently a protecting group;
(c) removing the protecting groups PG and PG′ under suitable reaction conditions;
(d) reacting the product of step (a) the product of step (c);
(e) performing an oxidative cleavage to provide a 1,2-dicarbonyl species comprising the repeat unit Formula (IIe):

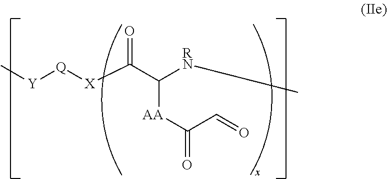

wherein x is as defined above;
reacting the product of step (e) with a linker moiety H-L²-LG, wherein L² and LG are as defined above; and
(g) reacting the product of step (f) with a biologically active molecule B—H.

Alternatively, Z is a group of formula (ii), and the method comprises the steps of:
(a) reacting an antibody or antigen-binding fragment thereof with a polymer-antibody linker;
(b) separately, reacting a compound of Formula (IId):

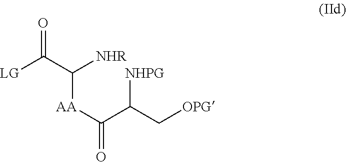

with a compound of Formula (IIb):

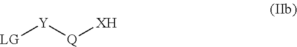

wherein Q, R, X, Y, AA and LG are as defined above, and PG and PG′ are each independently a protecting group;
(c) removing the protecting groups PG and PG′ under suitable reaction conditions;
(d) performing an oxidative cleavage to provide a 1,2-dicarbonyl species comprising the repeat unit Formula (IIe):

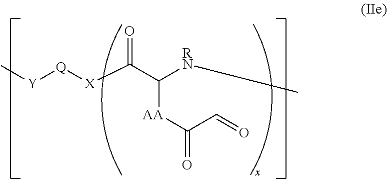

wherein x is as defined above;
(e) reacting the product of step (a) with the product of step (d);
(f) reacting the product of step (e) with a linker moiety H-L²-LG, wherein L² and LG are as defined above; and
(g) reacting the product of step (f) with a biologically active molecule B—H.

Alternatively, Z is a group of formula (ii), and the method comprises the steps of:
(a) reacting an antibody or antigen-binding fragment thereof with a polymer-antibody linker;
(b) separately, reacting a compound of Formula (IId):

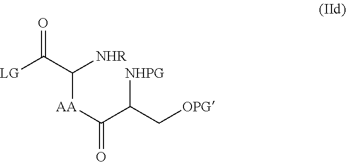

with a compound of Formula (IIb):

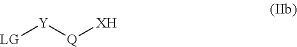

wherein Q, R, X, Y, AA and LG are as defined above, and PG and PG′ are each independently a protecting group;
(c) removing the protecting groups PG and PG′ under suitable reaction conditions;
(d) reacting the product of step (a) with the product of step (c);
(e) performing an oxidative cleavage to provide a 1,2-dicarbonyl species comprising the repeat unit Formula (IIe):

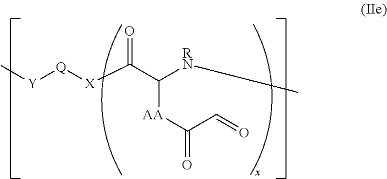

wherein x is as defined above;
(f) separately, reacting a linker moiety H-L²-LG, wherein L² and LG are as defined above, with a biologically active molecule B—H; and
(g) reacting the product of step (e) with the product of step (f).

Alternatively, Z is a group of formula (ii), and the method comprises the steps of:
(a) reacting an antibody or antigen-binding fragment thereof with a polymer-antibody linker;
(b) separately, reacting a compound of Formula (IId):

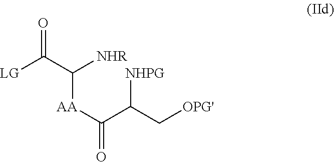

with a compound of Formula (IIb):

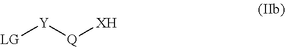

wherein Q, R, X, Y, AA and LG are as defined above, and PG and PG′ are each independently a protecting group;
(c) removing the protecting groups PG and PG′ under suitable reaction conditions;
(d) performing an oxidative cleavage to provide a 1,2-dicarbonyl species comprising the repeat unit Formula (IIe):

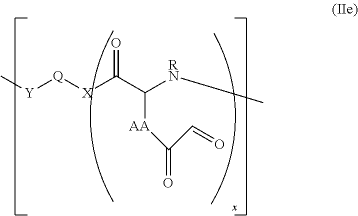

wherein x is as defined above;
(e) reacting the product of step (a) with the product of step (d);
(f) separately, reacting a linker moiety H-L²-LG, wherein L² and LG are as defined above, with a biologically active molecule B—H; and
(g) reacting the product of step (e) with the product of step (f).

Alternatively, Z is a group of formula (ii), and the method comprises the steps of:
(a) reacting an antibody or antigen-binding fragment thereof with a polymer-antibody linker;
(b) separately, reacting a compound of Formula (IId):

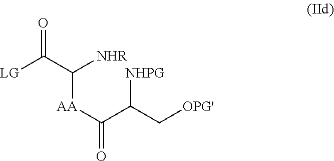

with a compound of Formula (IIb):

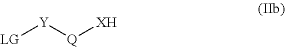

wherein Q, R, X, Y, AA and LG are as defined above, and PG and PG′ are each independently a protecting group;
(c) removing the protecting groups PG and PG′ under suitable reaction conditions;
(d) performing an oxidative cleavage to provide a 1,2-dicarbonyl species comprising the repeat unit Formula (IIe):

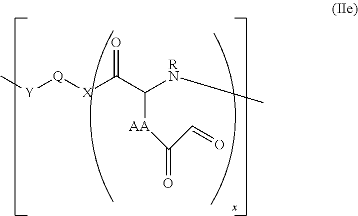

wherein x is as defined above;
(e) reacting the product of step (d) with a linker moiety H-L²-LG, wherein L² and LG are as defined above; and
(f) reacting the product of step (e) with a biologically active molecule B—H; and
(g) reacting the product of step (a) with the product of step (f).

Alternatively, Z is a group of formula (ii), and the method comprises the steps of:
(a) reacting an antibody or antigen-binding fragment thereof with a polymer-antibody linker;
(b) separately, reacting a compound of Formula (IId):

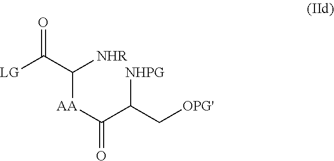

with a compound of Formula (IIb):

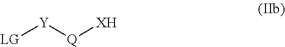

wherein Q, R, X, Y, AA and LG are as defined above, and PG and PG′ are each independently a protecting group;
(c) removing the protecting groups PG and PG′ under suitable reaction conditions;
(d) performing an oxidative cleavage to provide a 1,2-dicarbonyl species comprising the repeat unit Formula (IIe):

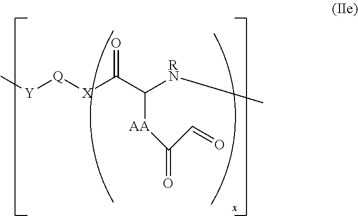

wherein x is as defined above;
(e) separately, reacting a linker moiety H-L²-LG, wherein L² and LG are as defined above, with a biologically active molecule B—H; and
(f) reacting the product of step (d) with the product of step (e); and
(g) reacting the product of step (f) with the product of step (a).

Alternatively, Z is a group of formula (iii), and the method comprises the steps of:
(a) reacting a compound of Formula (IIf):

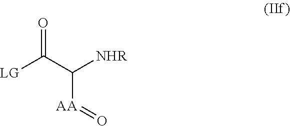

with a compound of Formula (IIb):

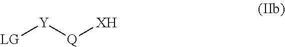

wherein Q, R, X, Y, AA and LG are as defined above;
(b) reacting the product of step (a) with a polymer-antibody linker;
(c) reacting the product of step (b) with a linker moiety H-L²-LG, wherein L² and LG are as defined above;
(d) reacting the product of step (c) with a biologically active molecule B—H; and
(e) reacting the product of step (d) with an antibody or antigen-binding fragment thereof.

Alternatively, Z is a group of formula (iii), and the method comprises the steps of:
(a) reacting a compound of Formula (IIf):

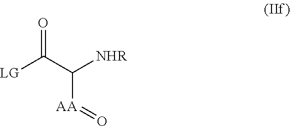

with a compound of Formula (IIb):

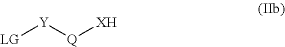

wherein Q, R, X, Y, AA and LG are as defined above;
(b) reacting the product of step (a) with a linker moiety H-L²-LG, wherein L² and LG are as defined above;
(c) reacting the product of step (b) with a biologically active molecule B—H;
(d) reacting the product of step (c) with a polymer-antibody linker; and
(e) reacting the product of step (d) with an antibody or antigen-binding fragment thereof.

Alternatively, Z is a group of formula (iii), and the method comprises the steps of:
(a) reacting a compound of Formula (IIf):

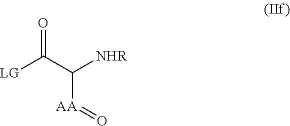

with a compound of Formula (IIb):

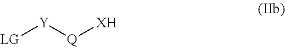

wherein Q, R, X, Y, AA and LG are as defined above;
(b) reacting the product of step (a) with a polymer-antibody linker;
(c) separately, reacting a linker moiety H-L²-LG, wherein L² and LG are as defined above, with a biologically active molecule B—H;
(d) reacting the product of step (b) with the product of step (c); and
(e) reacting the product of step (d) with an antibody or antigen-binding fragment thereof.

Alternatively, Z is a group of formula (iii), and the method comprises the steps of:
(a) reacting a compound of Formula (IIf):

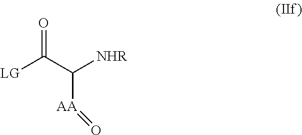

with a compound of Formula (IIb):

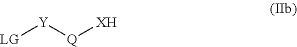

wherein Q, R, X, Y, AA and LG are as defined above;
(b) separately, reacting a linker moiety H-L²-LG, wherein L² and LG are as defined above, with a biologically active molecule B—H;
(c) reacting the product of step (a) with the product of step (b);
(d) reacting the product of step (c) with a polymer-antibody linker; and
(e) reacting the product of step (d) with an antibody or antigen-binding fragment thereof.

Alternatively, Z is a group of formula (iii), and the method comprises the steps of:
(a) reacting an antibody or antigen-binding fragment thereof with a polymer-antibody linker;
(b) separately, reacting a compound of Formula (IIf):

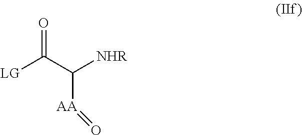

with a compound of Formula (IIb):

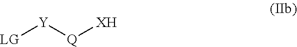

wherein Q, R, X, Y, AA and LG are as defined above;
(c) reacting the product of step (a) with the product of step (b);
(d) reacting the product of step (c) with a linker moiety H-L²-LG, wherein L² and LG are as defined above;
(e) reacting the product of step (d) with a biologically active molecule B—H.

Alternatively, Z is a group of formula (iii), and the method comprises the steps of:
(a) reacting an antibody or antigen-binding fragment thereof with a polymer-antibody linker;
(b) separately, reacting a compound of Formula (IIf):

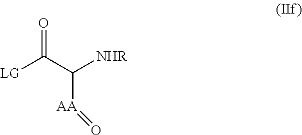

with a compound of Formula (IIb):

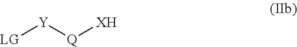

wherein Q, R, X, Y, AA and LG are as defined above;
(c) reacting the product of step (b) with a linker moiety H-L²-LG, wherein L² and LG are as defined above;
(d) reacting the product of step (c) with a biologically active molecule B—H; and
(e) reacting the product of step (a) with the product of step (d).

Alternatively, Z is a group of formula (iii), and the method comprises the steps of:
(a) reacting an antibody or antigen-binding fragment thereof with a polymer-antibody linker;
(b) separately, reacting a compound of Formula (IIf):

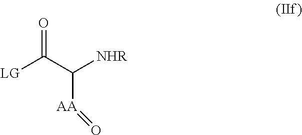

with a compound of Formula (IIb):

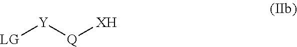

wherein Q, R, X, Y, AA and LG are as defined above;
(c) reacting the product of step (a) with the product of step (b);
(d) separately, reacting a linker moiety H-L²-LG, wherein L² and LG are as defined above, with a biologically active molecule B—H; and
(e) reacting the product of step (c) with the product of step (d).

Alternatively, Z is a group of formula (iii), and the method comprises the steps of:
(a) reacting an antibody or antigen-binding fragment thereof with a polymer-antibody linker;
(b) separately, reacting a compound of Formula (IIf):

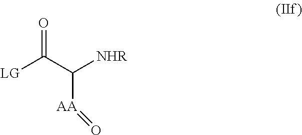

with a compound of Formula (IIb):

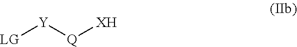

wherein Q, R, X, Y, AA and LG are as defined above;
(c) separately, reacting a linker moiety H-L²-LG, wherein L² and LG are as defined above, with a biologically active molecule B—H;
(d) reacting the product of step (b) with the product of step (c); and
(e) reacting the product of step (a) with the product of step (d).

Alternatively, Z is a group of formula (iv), and the method comprises the steps of:
(a) reacting a compound of Formula (IIg) or Formula (IIh):

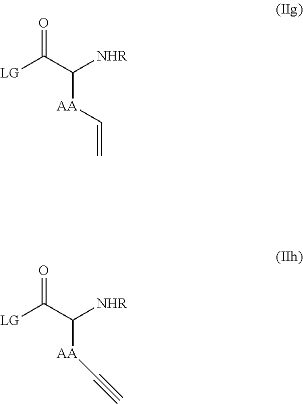

with a compound of Formula (IIb):

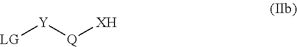

wherein Q, R, X, Y, AA and LG are as defined above;
(b) reacting the product of step (a) with a polymer-antibody linker;
(c) reacting the product of step (b) with a linker moiety N₃-L³-LG, wherein L³ and LG are as defined above;
(d) reacting the product of step (c) with a biologically active molecule B—H; and
(e) reacting the product of step (d) with an antibody or antigen-binding fragment thereof.

Alternatively, Z is a group of formula (iv), and the method comprises the steps of:
(a) reacting a compound of Formula (IIg) or Formula (IIh):

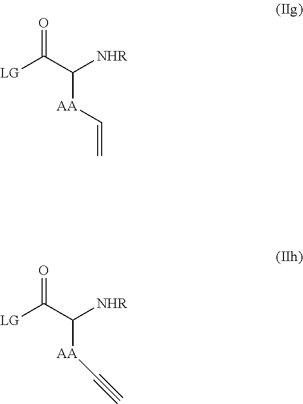

with a compound of Formula (IIb):

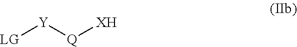

wherein Q, R, X, Y, AA and LG are as defined above;
(b) reacting the product of step (a) with a linker moiety N₃-L³-LG, wherein L³ and LG are as defined above;
(c) reacting the product of step (b) with a biologically active molecule B—H;
(d) reacting the product of step (c) with a polymer-antibody linker; and
(e) reacting the product of step (d) with an antibody or antigen-binding fragment thereof.

Alternatively, Z is a group of formula (iv), and the method comprises the steps of:
(a) reacting a compound of Formula (IIg) or Formula (IIh):

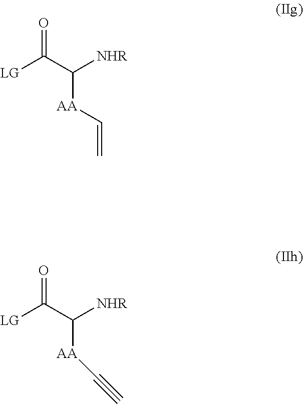

with a compound of Formula (IIb):

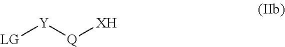

wherein Q, R, X, Y, AA and LG are as defined above;
(b) reacting the product of step (a) with a polymer-antibody linker;
(c) separately, reacting a linker moiety N₃-L³-LG, wherein L³ and LG are as defined above, with a biologically active molecule B—H;
(d) reacting the product of step (b) with the product of step (c); and
(e) reacting the product of step (d) with an antibody or antigen-binding fragment thereof.

Alternatively, Z is a group of formula (iv), and the method comprises the steps of:
(a) reacting a compound of Formula (IIg) or Formula (IIh):

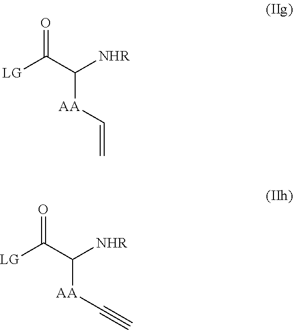

with a compound of Formula (IIb):

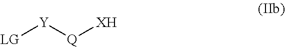

wherein Q, R, X, Y, AA and LG are as defined above;
(b) separately, reacting a linker moiety N₃-L³-LG, wherein L³ and LG are as defined above, with a biologically active molecule B—H;
(c) reacting the product of step (a) with the product of step (b);
(d) reacting the product of step (c) with a polymer-antibody linker; and
(e) reacting the product of step (d) with an antibody or antigen-binding fragment thereof.

Alternatively, Z is a group of formula (iv), and the method comprises the steps of:
(a) reacting an antibody or antigen-binding fragment thereof with a polymer-antibody linker;
(b) separately, reacting a compound of Formula (IIg) or Formula (IIh):

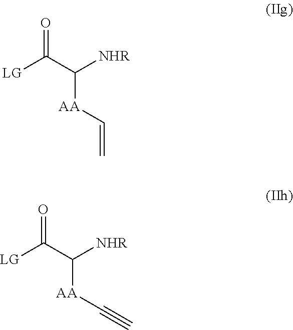

with a compound of Formula (IIb):

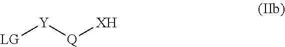

wherein Q, R, X, Y, AA and LG are as defined above;
(c) reacting the product of step (a) with the product of step (b);
(d) reacting the product of step (c) with a linker moiety N₃-L³-LG, wherein L³ and LG are as defined above;
(e) reacting the product of step (d) with a biologically active molecule B—H.

Alternatively, Z is a group of formula (iv), and the method comprises the steps of:
(a) reacting an antibody or antigen-binding fragment thereof with a polymer-antibody linker;
(b) separately, reacting a compound of Formula (IIg) or Formula (IIh):

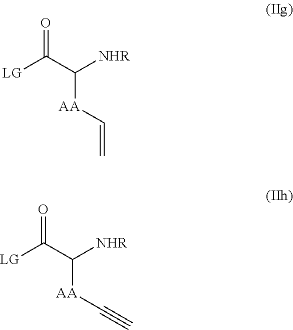

with a compound of Formula (IIb):

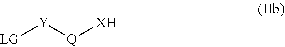

wherein Q, R, X, Y, AA and LG are as defined above;
(c) reacting the product of step (b) with a linker moiety N₃-L³-LG, wherein L³ and LG are as defined above;
(d) reacting the product of step (c) with a biologically active molecule B—H; and
(e) reacting the product of step (a) with the product of step (d).

Alternatively, Z is a group of formula (iv), and the method comprises the steps of:
(a) reacting an antibody or antigen-binding fragment thereof with a polymer-antibody linker;
(b) separately, reacting a compound of Formula (IIg) or Formula (IIh):

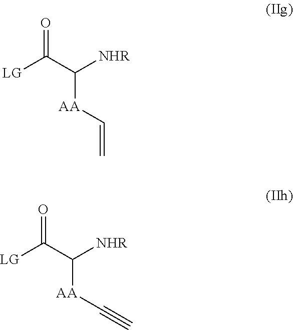

with a compound of Formula (IIb):

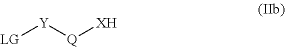

wherein Q, R, X, Y, AA and LG are as defined above;
(c) reacting the product of step (a) with the product of step (b);
(d) separately, reacting a linker moiety N₃-L³-LG, wherein L³ and LG are as defined above, with a biologically active molecule B—H; and
(e) reacting the product of step (c) with the product of step (d).

Alternatively, Z is a group of formula (iv), and the method comprises the steps of:
(a) reacting an antibody or antigen-binding fragment thereof with a polymer-antibody linker;
(b) separately, reacting a compound of Formula (IIg) or Formula (IIh):

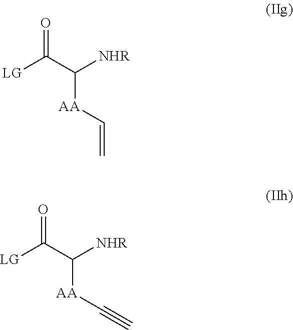

with a compound of Formula (IIb):

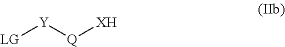

wherein Q, R, X, Y, AA and LG are as defined above;
(c) separately, reacting a linker moiety N₃-L³-LG, wherein L³ and LG are as defined above, with a biologically active molecule B—H;
(d) reacting the product of step (b) with the product of step (c); and
(e) reacting the product of step (a) with the product of step (d).

Alternatively, Z is a group of formula (v), and the method comprises the steps of:
(a) reacting a compound of Formula (IIj):

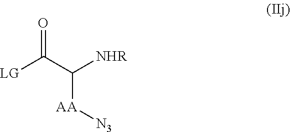

with a compound of Formula (IIb):

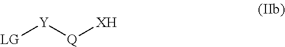

wherein Q, R, X, Y, AA and LG are as defined above;
(b) reacting the product of step (a) with a polymer-antibody linker;
(c) reacting the product of step (b) with a linker moiety HC≡C-L³-LG or H₂C═CH-L³-LG, wherein L³ and LG are as defined above;
(d) reacting the product of step (c) with a biologically active molecule B—H; and
(e) reacting the product of step (d) with an antibody or antigen-binding fragment thereof.

Alternatively, Z is a group of formula (v), and the method comprises the steps of:
(a) reacting a compound of Formula (IIj):

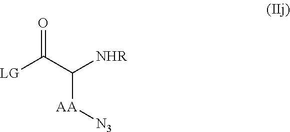

with a compound of Formula (IIb):

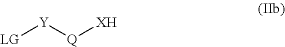

wherein Q, R, X, Y, AA and LG are as defined above;
(b) reacting the product of step (a) with a linker moiety HC≡C-L³-LG or H₂C═CH-L³-LG, wherein L³ and LG are as defined above;
(c) reacting the product of step (b) with a biologically active molecule B—H;
(d) reacting the product of step (c) with a polymer-antibody linker; and
(e) reacting the product of step (d) with an antibody or antigen-binding fragment thereof.

Alternatively, Z is a group of formula (v), and the method comprises the steps of:
(a) reacting a compound of Formula (IIj):

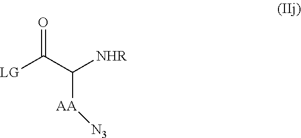

with a compound of Formula (IIb):

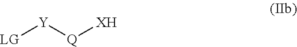

wherein Q, R, X, Y, AA and LG are as defined above;
(b) reacting the product of step (a) with a polymer-antibody linker;
(c) separately, reacting a linker moiety HC≡C-L³-LG or H₂C═CH-L³-LG, wherein L³ and LG are as defined above, with a biologically active molecule B—H;
(d) reacting the product of step (b) with the product of step (c); and
(e) reacting the product of step (d) with an antibody or antigen-binding fragment thereof.

Alternatively, Z is a group of formula (v), and the method comprises the steps of:
(a) reacting a compound of Formula (IIj):

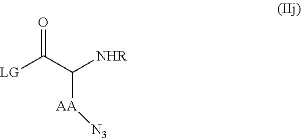

with a compound of Formula (IIb):

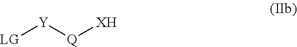

wherein Q, R, X, Y, AA and LG are as defined above;
(b) separately, reacting a linker moiety HC≡C-L³-LG or H₂C═CH-L³-LG, wherein L³ and LG are as defined above, with a biologically active molecule B—H;
(c) reacting the product of step (a) with the product of step (b);
(d) reacting the product of step (c) with a polymer-antibody linker; and
(e) reacting the product of step (d) with an antibody or antigen-binding fragment thereof.

Alternatively, Z is a group of formula (v), and the method comprises the steps of:
(a) reacting an antibody or antigen-binding fragment thereof with a polymer-antibody linker;
(b) separately, reacting a compound of Formula (IIj):

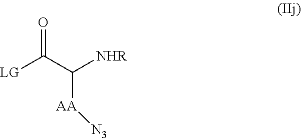

with a compound of Formula (IIb):

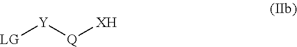

wherein Q, R, X, Y, AA and LG are as defined above;
(c) reacting the product of step (a) with the product of step (b);
(d) reacting the product of step (c) with a linker moiety HC≡C-L³-LG or H₂C═CH-L³-LG, wherein L³ and LG are as defined above;
(e) reacting the product of step (d) with a biologically active molecule B—H.

Alternatively, Z is a group of formula (v), and the method comprises the steps of:
(a) reacting an antibody or antigen-binding fragment thereof with a polymer-antibody linker;
(b) separately, reacting a compound of Formula (IIj):

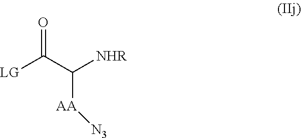

with a compound of Formula (IIb):

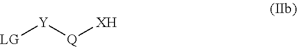

wherein Q, R, X, Y, AA and LG are as defined above;
(c) reacting the product of step (b) with a linker moiety HC≡C-L³-LG or H₂C═CH-L³-LG, wherein L³ and LG are as defined above;
(d) reacting the product of step (c) with a biologically active molecule B—H; and
(e) reacting the product of step (a) with the product of step (d).

Alternatively, Z is a group of formula (v), and the method comprises the steps of:
(a) reacting an antibody or antigen-binding fragment thereof with a polymer-antibody linker;
(b) separately, reacting a compound of Formula (IIj):

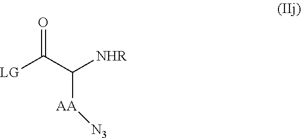

with a compound of Formula (IIb):

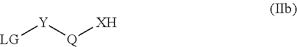

wherein Q, R, X, Y, AA and LG are as defined above;
(c) reacting the product of step (a) with the product of step (b);
(d) separately, reacting a linker moiety HC≡C-L³-LG or H₂C═CH-L³-LG, wherein L³ and LG are as defined above, with a biologically active molecule B—H; and
(e) reacting the product of step (c) with the product of step (d).

Alternatively, Z is a group of formula (v), and the method comprises the steps of:
(a) reacting an antibody or antigen-binding fragment thereof with a polymer-antibody linker;
(b) separately, reacting a compound of Formula (IIj):

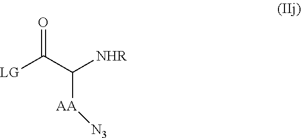

with a compound of Formula (IIb):

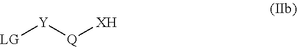

wherein Q, R, X, Y, AA and LG are as defined above;
(c) separately, reacting a linker moiety HC≡C-L³-LG or H₂C═CH-L³-LG, wherein L³ and LG are as defined above, with a biologically active molecule B—H;
(d) reacting the product of step (b) with the product of step (c); and
(e) reacting the product of step (a) with the product of step (d).

In a particularly preferred method, Z is a group of formula (ii) and the method comprises the steps of:
(a) reacting a compound of Formula (IId):

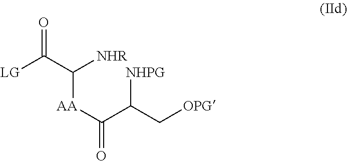

with a compound of Formula (IIb):

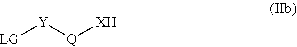

wherein Q, R, X, Y, AA and LG are as defined above, and PG and PG′ are each independently a protecting group;
(b) reacting the product of step (a) with a polymer-antibody linker;
(c) removing the protecting groups PG and PG′ under suitable reaction conditions;
(d) performing an oxidative cleavage to provide a 1,2-dicarbonyl species comprising the repeat unit Formula (IIe):

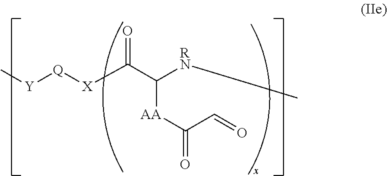

wherein x is as defined above;
(e) separately, reacting a linker moiety H-L²-LG, wherein L² and LG are as defined above, with a biologically active moiety B—H;
(f) reacting the product of step (d) with the product of step (e); and
(g) reacting the product of step (f) with an antibody or antigen-binding fragment thereof.

In preferred methods of the invention, the biologically active molecule is as defined herein or a protected version of a biologically active molecule as defined herein. Conventional protecting group strategies, as are well known in the art, may be employed during the polymerisation, functionalization and conjugation reactions. In further preferred methods of the invention, the antibody is as defined herein. In yet further preferred methods of the invention, the polymer-antibody linker moiety is as defined herein.

In particularly preferred methods wherein Z is a group of formula (ii), PG is any suitable amine protecting group. Preferably, PG is an acetal, benzoyl, tosyl, para-methyoxybenzyl, sulfonamide, or carbamate protecting group. Non-limiting examples of carbamate protecting groups include tert-butyloxycarbonyl (Boc), carboxybenyl (Cbz), or fluorenylmethyloxycarbonyl (Fmoc). In particularly preferred methods wherein Z is a group of formula (ii), PG′ is any suitable alcohol protecting group. Preferably, PG′ is an acetyl, benzoyl, benzyl, P-methoxyethoxymethyl ether (MEM), methoxymethyl ether (MOM), para-methoxybenzyl ether (PMB), pivaloyl (Piv), tetrahydropyranyl (THP), tetrahydrofuran (THF), trityl (Tr), silyl ether or ester protecting group. A particularly preferred protecting group PG′ is a tert-butyl ester. In some particularly preferred methods, PG and PG′ are cleaved under the same reaction conditions. Alternatively, in some methods, PG and PG′ are cleaved under orthogonal reaction conditions. In one particularly preferred method, PG is Boc and PG′ is tert-butyl ester. These groups may be simultaneously cleaved by the addition of acid, e.g. trifluoroacetic acid (TFA).

The polymerisation step in the methods of the invention is preferably carried out enzymatically, by solid phase peptide synthesis (SPPS), by polycondensation, by free radical chain growth polymerisation or by ring-opening polymerisation, most preferably enzymatically or by SPPS.

Any step in any method above that involves reacting a molecule H-L²-LG, HC≡C-L³-LG, H₂C═CH-L³-LG or N₃-L³-LG with a biologically active molecule B—H, can be replaced with any suitable alternative for creating the respective molecules H-L²-B, HC≡C-L³-B, H₂C═CH-L³-B or N₃-L³-B. This may include the condensation of two units to form a bond within the linker moiety L² or L³ as the final synthetic step. For example, when Z in the target product is a group of formula (ii) or (iii), a molecule H—V³-LG may be reacted with a molecule H-L′-V²—B to make a molecule H-L²-B. For instance, in a preferable method, a molecule H—V³—OH may be reacted with a molecule H—Val-Cit-PAB-(C═O)—B in order to form H-L²-B. Likewise, when Z in the target product is a group of formula (iv), a molecule N₃—V⁴-LG may be reacted with a molecule H-L′-V²—B to make a molecule N₃-L³-LG. Likewise, when Z in the target product is a group of formula (v), a molecule HC≡C—V⁴-LG or H₂C═CH—V⁴-LG may be reacted with a molecule H-L′-V²—B to make a molecule HC≡C-L³-LG.

Pharmaceutical Compositions

The antibody-drug conjugates of the present invention may be incorporated into pharmaceutical compositions. Thus, the present invention provides a pharmaceutical composition comprising an antibody-drug conjugate as defined herein, and one or more pharmaceutically acceptable carriers, diluents or excipients. Pharmaceutical compositions may be prepared in any conventional manner. A pharmaceutical composition may comprise one or more different antibody-drug conjugates as described herein. Suitable carriers, diluents and excipients are well known in the art.

Pharmaceutical compositions of the invention may be administered to a patient by any one or more of the following routes: oral, systemic (e.g. transdermal, intranasal, transmucosal or by suppository), or parenteral (e.g. intramuscular, intravenous or subcutaneous). Compositions of the invention can take the form of tablets, pills, capsules, semisolids, powders, sustained release formulations, solutions, suspensions, elixirs, aerosols, transdermal patches, bioadhesive films, or any other appropriate compositions. The choice of formulation depends on various factors such as the mode of drug administration (e.g. for oral administration, formulations in the form of tablets, pills or capsules are preferred) and the bioavailability of the drug substance.

The pharmaceutical compositions of the invention may additionally include common pharmaceutical excipients such as lubricating agents, thickening agents, wetting agents, emulsifying agents, suspending agents, preserving agents, fillers, binders, preservatives and adsorption enhancers, e.g. surface penetrating agents. Solubilizing and/or stabilizing agents may also be used, e.g. cyclodextrins (CD). A person skilled in the art will be able to select suitable excipients based on their purpose. Common excipients that may be used in the pharmaceutical products herein described are listed in various handbooks (e.g. D. E. Bugay and W. P. Findlay (Eds) Pharmaceutical excipients (Marcel Dekker, New York, 999), E-M Hoepfner, A. Reng and P. C. Schmidt (Eds) Fiedler Encyclopedia of Excipients for Pharmaceuticals, Cosmetics and Related Areas (Edition Cantor, Munich, 2002) and H. P. Fielder (Ed) Lexikon der Hilfsstoffe fur Pharmazie, Kosmetik and angrenzende Gebiete (Edition Cantor Aulendorf, 1989)).

The pharmaceutical compositions of the invention may be formulated so as to provide quick, sustained or delayed release of the antibody-drug conjugate after administration to the patient by employing procedures well known in the art. The concentration of the antibody-drug conjugates in the pharmaceutical compositions depends upon numerous factors including the nature of the polymer, the drug loading on the polymer, the identity of the antibody, the composition, the mode of administration, the condition to be treated or diagnosed, and the subject to which it is administered and may be varied or adjusted according to choice by techniques well-known to a person of skill in the art.

Medical Uses of the Antibody-Drug Conjugates

The antibody-drug conjugates and pharmaceutical compositions described herein are useful in medical applications. Thus, the present invention provides an antibody-drug conjugate as described herein for use in the treatment of a disease or condition in a patient in need thereof. Typically, the antibody-drug conjugates and pharmaceutical compositions described herein are for use in the treatment of a disease selected from inflammatory diseases (e.g. inflammatory bowel disease, rheumatoid arthritis and artherosclerosis), metabolic disorders (e.g. diabetes, insulin resistance, obesity), cancer, bacterial infections (e.g. Tuberculosis, pneumonia, endocarditis, septicaemia, salmonellosis, typhoid fever, cystic fibrosis, chronic obstructive pulmonary diseases), viral infections, cardiovascular diseases, neurodegenerative diseases, neurological disorders, behavioural and mental disorders, blood diseases, chromosome disorders, congenital and genetic diseases, connective tissue diseases, digestive diseases, ear, nose, and throat diseases, endocrine diseases, environmental diseases, eye diseases, female reproductive diseases, fungal infections, heart diseases, hereditary cancer syndromes, immune system diseases, kidney and urinary diseases, lung diseases, male reproductive diseases, mouth diseases, musculoskeletal diseases, myelodysplastic syndromes, nervous system diseases, newborn screening, nutritional diseases, parasitic diseases, rare cancers, and skin diseases.

In general, antibody-drug conjugates of the present invention are administered to a human patient so as to deliver to the patient a therapeutically effective amount of the biologically active molecule contained therein.

As used herein, the term “therapeutically effective amount” refers to an amount of the biologically active molecule which is sufficient to reduce or ameliorate the severity, duration, progression, or onset of a disorder being treated, prevent the advancement of a disorder being treated, cause the regression of, prevent the recurrence, development, onset or progression of a symptom associated with a disorder being treated, or enhance or improve the prophylactic or therapeutic effect(s) of another therapy. The precise amount of biologically active molecule administered to a patient will depend on the type and severity of the disease or condition and on the characteristics of the patient, such as general health, age, sex, body weight and tolerance to drugs. It will also depend on the degree, severity and type of the disorder being treated. The skilled artisan will be able to determine appropriate dosages depending on these and other factors.

As used herein, the terms “treat”, “treatment” and “treating” refer to the reduction or amelioration of the progression, severity and/or duration of a disorder being treated, or the amelioration of one or more symptoms (preferably, one or more discernible symptoms) of a disorder being treated resulting from the administration of a film according to the invention to a patient.

The present invention also provides a method of treating a disease or condition as described herein in a human patient, wherein said method comprises administration of at least one antibody-drug conjugate as described herein to a patient in need thereof.

The present invention also provides the use of an antibody-drug conjugate as described herein for the manufacture of a medicament for the treatment of a disease or condition as described herein in a human patient.

Any antibody-drug conjugate or antibody-drug conjugates of the present invention may also be used in combination with one or more other drugs or pharmaceutical compositions in the treatment of disease or conditions for which the ADCs of the present invention and/or the other drugs or pharmaceutical compositions may have utility.

The one or more other drugs or pharmaceutical compositions may be administered to the patient by any one or more of the following routes: oral, systemic (e.g. transdermal, intranasal, transmucosal or by suppository), or parenteral (e.g. intramuscular, intravenous or subcutaneous). Compositions of the one or more other drugs or pharmaceutical compositions can take the form of tablets, pills, capsules, semisolids, powders, sustained release formulations, solutions, suspensions, elixirs, aerosols, transdermal patches, bioadhesive films, or any other appropriate compositions. The choice of formulation depends on various factors such as the mode of drug administration (e.g. for oral administration, formulations in the form of tablets, pills or capsules are preferred) and the bioavailability of the drug substance.

The publications, patent publications and other patent documents cited herein are entirely incorporated by reference. Herein, any reference to a term in the singular also encompasses its plural. Where the term “comprising”, “comprise” or “comprises” is used, said term may substituted by “consisting of”, “consist of” or “consists of” respectively, or by “consisting essentially of”, “consist essentially of” or “consists essentially of” respectively. Any reference to a numerical range or single numerical value also includes values that are about that range or single value. Any reference to a polymer having a repeat unit of Formula (I) also encompasses a physiologically acceptable salt thereof unless otherwise indicated. Unless otherwise indicated, any % value is based on the relative weight of the component or components in question.

### EXAMPLES

The following are Examples that illustrate the present invention. However, these Examples are in no way intended to limit the scope of the invention.

### Example 1

### Preparation of Polymer (1)

A target polymer of formula (1) (Scheme 1) was synthesised via the following synthetic steps. The polymer (1) was built from monomers (2) and (3) (Scheme 2) using Solid Phase Synthesis (SPS) to enable construction of a polymer of a specific number of units. The polymer can then be cleaved from the resin to afford the product as a monodisperse polymer.

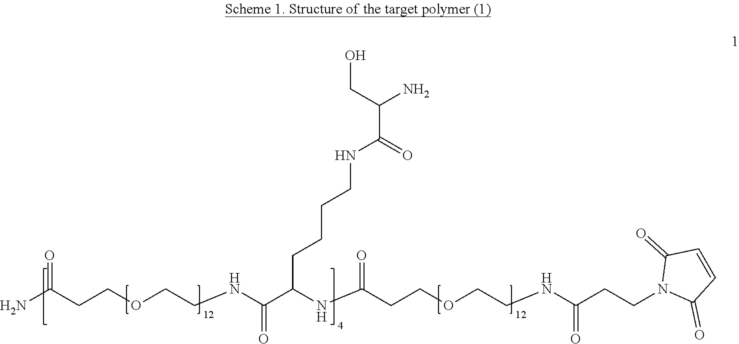

The Fmoc-protected PEG12-acid (2) was purchased from a commercial supplier and the amino acid derived monomer (3) was synthesised as described below. After building the polymer using SPS, the terminal amine group is capped by coupling with 3-maleimidopropionic acid, followed by a single cleavage and deprotection step using a cocktail of trifluoroacetic acid (TFA), triisopropylsilane (TIS) and water to release the polymer (1).

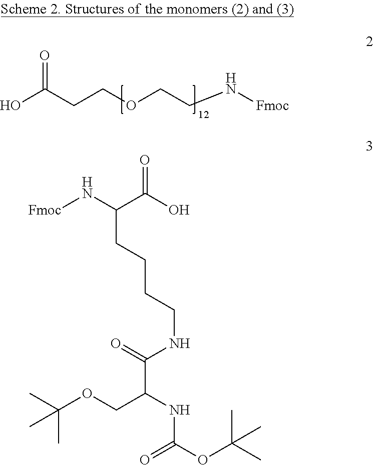

Step a: Preparation of Monomer (3)

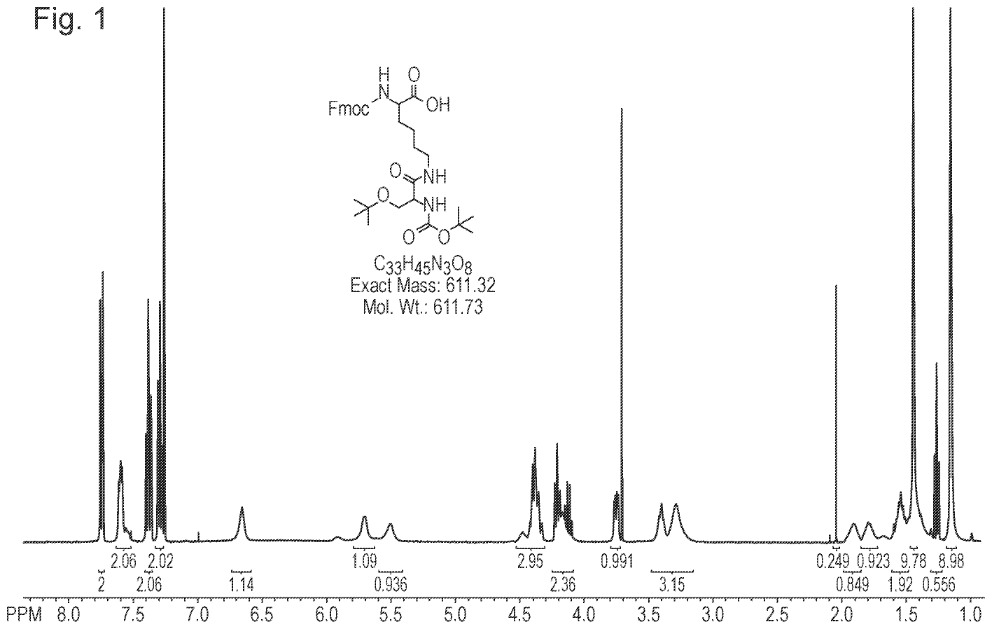

Boc-Ser(OtBu)-OH was activated by converting the acid group to the N-hydroxysuccinimide ester using DCC and N-hydroxysuccinimide in a mixture of ethyl acetate and 1,4-dioxane. The reaction resulted in 14.5 g of white solid from lOg of starting material (quantitative). The material was taken into the next step and reacted with Fmoc-Lys-OH.HCl in dichloromethane with diisopropyl ethylamine. The material isolated was a white solid with a 98% yield and the NMR showed the main product (3) (FIG. 1). HPLC analysis showed a purity of 90% at 214 nm and 95.2% at 254 nm.

Step b: Synthesis of Polymer (1) via SPS

The first step in the synthesis was an initial loading of the resin (750 mg) with the monomer (2), to achieve a loading of 0.3-0.4 mmol/g. A resin loading measurement by Fmoc cleavage was used in order to approximate the amount of substitution on the resin (0.36 mmol/g). After the remaining unsubstituted amino sites were capped by acylation with acetic anhydride, the polymer was built up by performing standard Fmoc deprotections (20% piperidine in DMF) and alternating the coupling/activation step (HATU and DIPEA in DMF) between monomer (3) and monomer (2). The procedure was used to build up a 4-unit polymer. Analysis was carried out at each stage of the reaction sequence. UV spectroscopy was used to monitor the deprotection of the Fmoc group at each phase of the reaction sequence. The absence of amine functionality at each coupling/activation stage by a Kaiser test suggested that the reactions were proceeding to completion. This data coupled with analysis from mass spectrometry (MALDI-ToF and ESI-MS) indicated polymer growth.

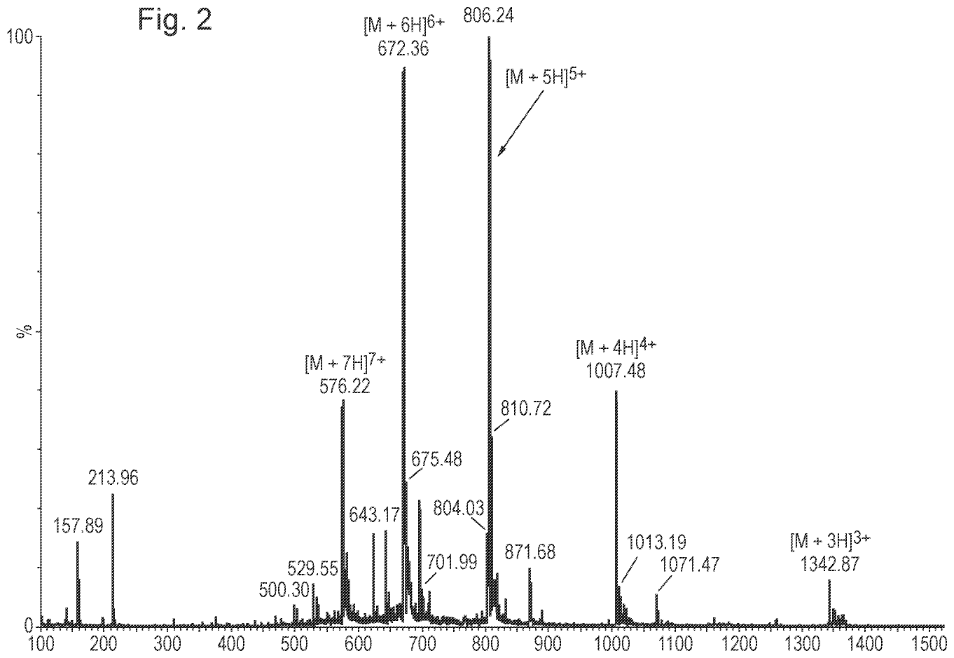

After building the polymer to 4-units, the amine was capped using a large excess of 3-maleimidopropionic acid using standard conditions, HATU as the coupling reagent and DIPEA as a base. A Kaiser test on the resin was negative for any amine residues, indicating complete capping of the polymer. Deprotection of the polymer and cleavage from the resin was performed, the crude residue obtained (1) was washed with diethyl ether and pentane. The polymer was dissolved in the minimum volume of DCM and pentane was added until the polymer came out of the solution. The organic solvent was removed carefully by pipette and this procedure was repeated. The residue was dissolved in DCM and the volatiles removed in vacuo at 35° C., the mass of crude (1) obtained was 852 mg. Polymer (1) was characterized by MS (FIG. 2).

### Example 2

### MMAE Drug Payload Attachment to the Polymer (1)

Step a: Synthesis of Polymer (4)

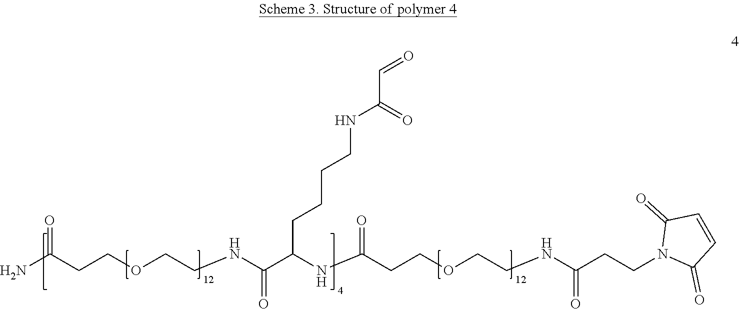

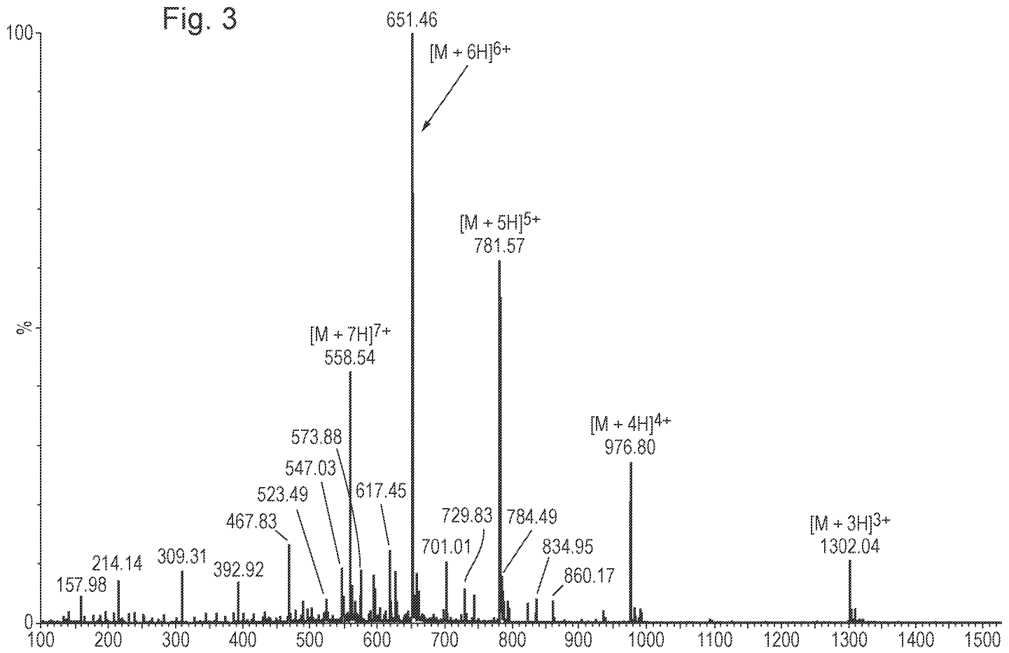

Oxidation with sodium periodate was performed on the crude polymer (1) to achieve the synthesis of polymer (4) (Scheme 3). To a solution of the crude polymer (1) (41 mg, 0.101 μmol) in mixture of Dulbecco's phosphate-buffered saline (712 μL) and acetonitrile (80 μL) was added NaIO₄ (40 mg, 187 μmol) as a solid in one portion. The reaction mixture was occasionally shaken over a period of 1 hour at ambient temperature. The reaction mixture was filtered by 0.45 μm, PTFE and purified immediately by prep-HPLC (C18) using a gradient of 15-45% MeCN in H₂O (0.05% TFA) over 35 min. Fractions were analysed by LC-MS and RP-UPLC. The fractions containing the desired product (4) were combined. Polymer (4) was characterized by MS (FIG. 3).

Step b: Synthesis of MMAE Reagent (5)

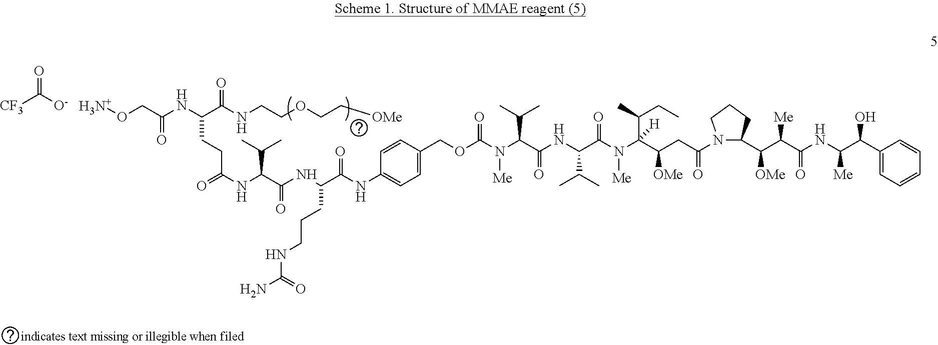

The synthesis of MMAE reagent (5) was achieved via the following steps.

### 1. Preparation of Fmoc-L-glutamide-(PEG₂₄-OMe)-γ-tert-butyl ester

A 40 mL vial with stir bar was charged with Fmoc-L-glutamic acid γ-tert-butyl ester α-N-hydroxysuccinimide ester and m-dPEG®₂₄-amine. DMF was then added via syringe and the material dissolved after agitation. DIPEA was then added via syringe and the contents agitated at room temperature for 2 hours. The reaction was quenched with 0.5 mL of AcOH and then the reaction mixture was concentrated to half the volume on a rotary evaporator. The crude reaction mixture was loaded onto a 150-gram ISCO Gold C18 column, equilibrated with 10% acetonitrile (ACN)/H₂O w/ 0.05% TFA. The material was eluted with ACN/H₂O w/ 0.05% TFA and fractions analyzed, collected, frozen and then lyophilized. After 2 days, the flask was removed from the lyophilizer to yield 2.62 grams (91.6% yield) of a white waxy solid.

### 2. Preparation of Fmoc-L-glutamide-(PEG₂₄-OMe)

In a 60 mL vial with stir bar was charged Fmoc-L-glutamide-(PEG₂₄-OMe)-γ-tert-butyl ester and DCM. The material dissolved by agitation and then cooled to 0 to −3° C. in an IPA/ice bath. TFA was then added via syringe over 15 minutes maintaining the temperature below 5° C. After complete addition of TFA, the contents were allowed to warm to room temperature and agitated for 1 hour. The vial contents were then concentrated on a rotary evaporator and the material used “as is” for subsequent transformations.

### 3. Preparation of Fmoc-L-glutamide-(PEG₂₄-OMe)-vc-PAB-MMAE

A 60 mL vial with stir bar was charged with vc-PAB-MMAE, HATU, Fmoc-L-glutamide-(PEG₂₄-OMe). DMF was added via syringe and the contents agitated to dissolve. Once a homogeneous solution was attained, DIPEA was added via syringe and the contents agitated at room temperature for 24 hours. The reaction was quenched with 1M aq. AcOH (10 mL) and then loaded onto a 275-gram ISCO Gold C18 column, equilibrated with 20% ACN/H₂O w/ 0.05% TFA. The material was eluted with ACN/H₂O w/ 0.05% TFA and fractions analyzed, collected, frozen and then lyophilized. After 5 days the flask was removed from the lyophilizer to yield 933 mg (65.4% yield) of a white solid.

### 4. Preparation of Boc-Aminooxyacetamide-L-glutamide-(PEG₂₄-OMe)-vc-PAB-MMAE

A 100 ml round bottom flask with stir bar was charged with Fmoc-L-glutamide-(PEG₂₄-OMe)-vc-PAB-MMAE and dissolved in methanol. Piperidine was then added via syringe and the contents agitated at room temperature for 18 hours. The reaction mixture was then concentrated on a rotary evaporator generating a solid. The concentrated reaction mixture was then dissolved in THF (20 mL) and then cooled in an ice bath to 2-5° C., followed by addition of DIPEA (3.5 mL). N-Boc-aminooxyacetic acid NHS ester was then added to the flask as a solid and the contents agitated at room temperature for 18 hours. The reaction mixture was then concentrated on a rotary evaporator then dissolved in DMF and acidified to pH 3 with 1 M HCl. The quenched mixture was then loaded onto a 150-gram ISCO Gold C18 column, equilibrated with 20% ACN/H₂O w/ 0.05% TFA. The material was eluted with ACN/H₂O w/ 0.05% TFA and fractions analyzed, collected, frozen and then lyophilized. After 5 days the flask was removed from the lyophilizer to yield 1.12 g (122% yield) of a clear glassy solid.

### 5. Preparation of Aminooxyacetamide-L-glutamide-(PEG₂₄-OMe)-vc-PAB-MMAE TFA (5)

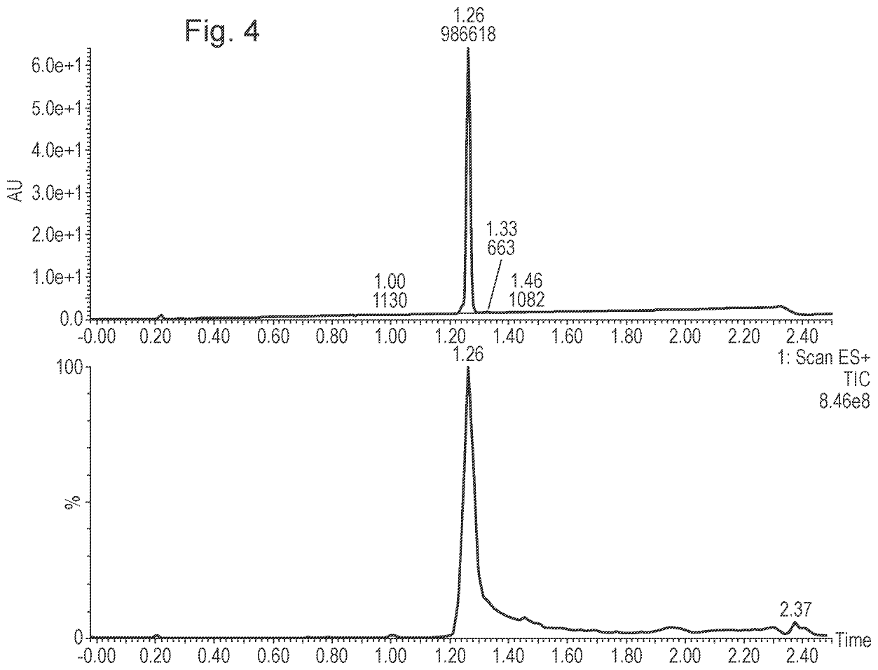

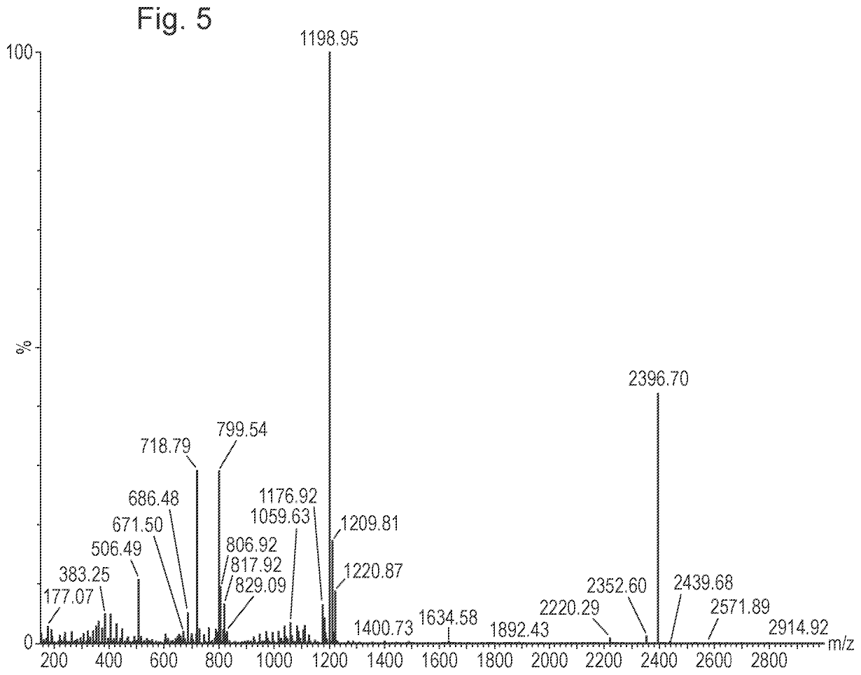

In a 100 mL round bottom flask with stir bar was charged Boc-Aminooxyacetamide-L-glutamide-(PEG₂₄-OMe)-vc-PAB-MMAE followed by addition of DCM (23 mL). The contents were agitated to dissolve and then the contents were cooled in an IPA/ice bath to −9.0° C. TFA was then added via syringe and the reaction mixture maintained between −9 and −14° C. for 5 hours. The reaction was quenched with 7 mL of N-methylmorpholine maintaining the temperature below 0° C. by controlled addition. The quenched mixture was then concentrated on a rotary evaporator at room temperature then dissolved in 2 mL of water. The solution was purified using an ISCO EZPrep instrument equipped with a 250×50 mm Luna C18 column equilibrated with 20% ACN/H₂O w/ 20 mmol NH₄OAc. The material was eluted with ACN/H₂O w/ 20 mmol NH₄OAc and fractions analyzed, collected, frozen and then lyophilized. After 3 days the flask was removed from the lyophilizer to yield 192 mg (34% yield) of a white solid of product (5) characterized by LC-MS (FIG. 4 and FIG. 5).

Step c: MMAE Reagent (5) Coupling to Polymer (4) to Generate MMAE Polymer Conjugate (6)

Oxime ligation was performed between the purified aldehyde-functionalised polymer (4) and hydroxylamine-vc-PAB-MMAE (5) to generate conjugate bearing 4 copies of drug payload MMAE (6) (Scheme 5).

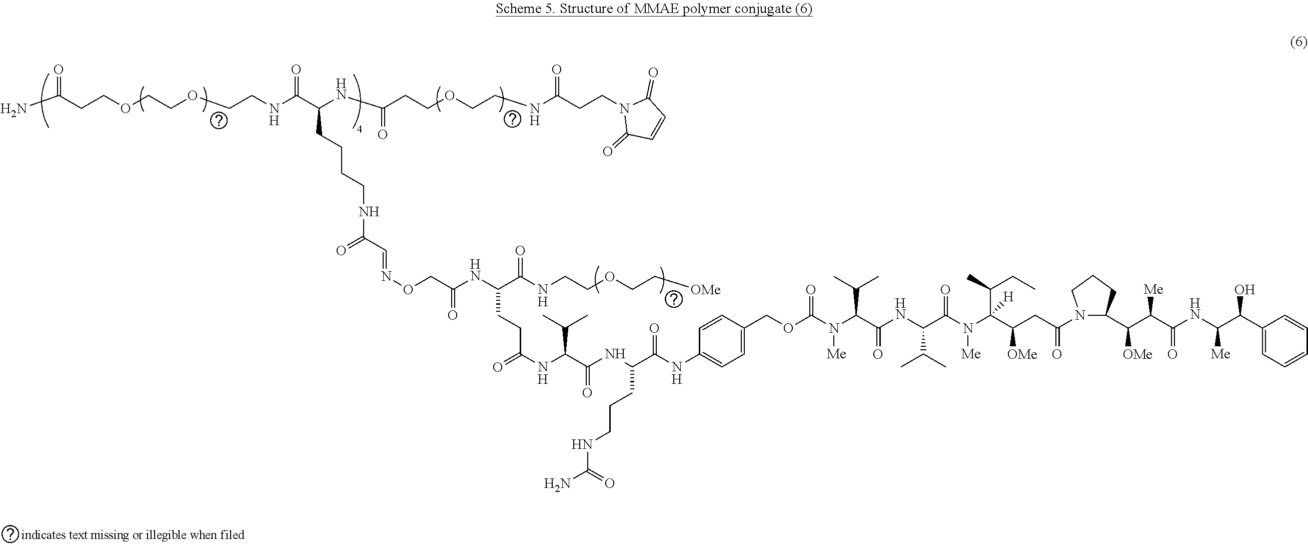

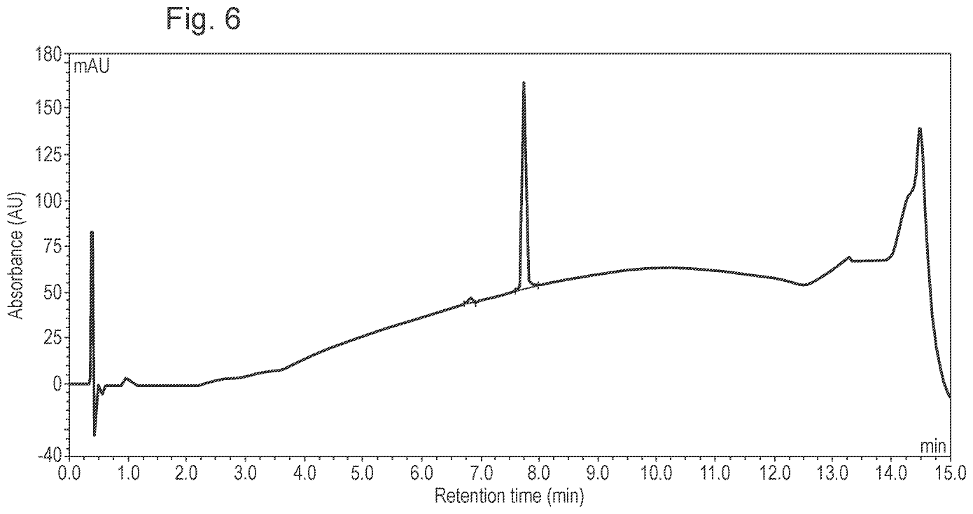

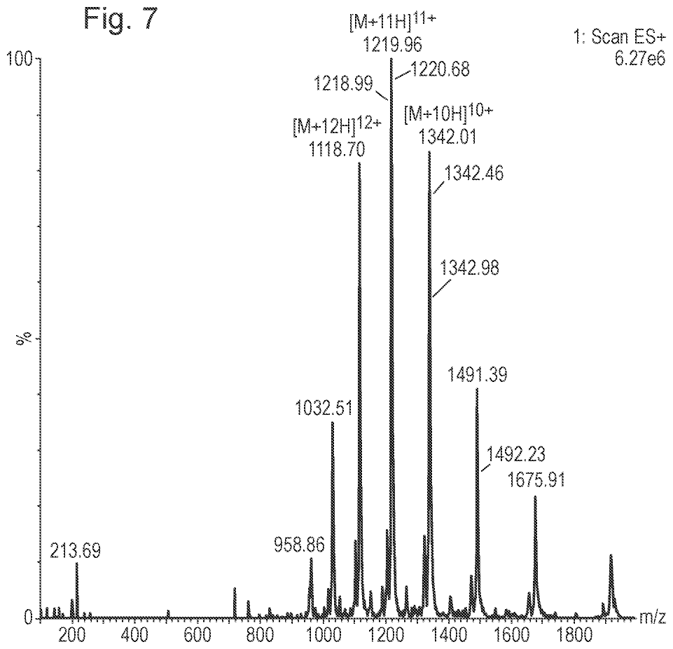

Aminooxyacetamide-L-glutamide-(PEG₂₄-OMe)-vc-PAB-MMAE TFA (5, 13 mg, 50.9 μmol) was dissolved in a mixture of MeCN:H₂O with 0.05% TFA, 1:1 v/v (250 μL) and added to the combined HPLC fractions of polymer (4). The resulting mixture was stirred at room temperature for 1 hour. Full conversion of the aldehyde polymer was observed by RP-UPLC analysis; the desired product formation confirmed by LC-MS. The reaction mixture was concentrated in vacuum and residue was directly purified by preparative RP-HPLC (C18) using a gradient of 30-80% MeCN in H₂O (0.05% TFA) over 25 min. Fractions of (6) were analysed by RP-UPLC and LC-MS (FIG. 6 and FIG. 7). The fractions containing the desired product were combined and lyophilized to give 6 mg of the desired product (6) as a white solid.

### Example 3

### MMAE ADC Preparation by Conjugation of MMAE Polymer Conjugate (6) to Trastuzumab

Trastuzumab at 10.6 mg/mL in reaction buffer: 20 mM sodium phosphate, pH 7.5, 150 mM NaCl, 20 mM EDTA (519 μL; 5.5 mg; 37 nmol; 1.0 eq.), was diluted with reaction buffer (381 μL) and warmed to 40° C. in a heating block for 10 min. A 5 mM solution of tris(2-carboxyethyl) phosphine hydrochloride (TCEP) in water was prepared by dilution from 0.5 M TCEP stock solution in water, pH 7, at 22° C., using endotoxin-free water. 5 mM TCEP solution (17.1 μL; 85.5 nmol; 2.3 eq.) was added to the trastuzumab solution at 40° C., resulting in a final trastuzumab concentration of 6 mg/mL. The trastuzumab solution was incubated at 40° C. for 2 h, after which it was allowed to cool down to 22° C.

A 26.0 mg/mL solution of MMAE polymer conjugate (6) in dimethyl sulfoxide (DMSO) was prepared by dissolving 6.0 mg of (6) (MW=13415 g.mol⁻¹) in 231 μL of DMSO. The (6) reagent solution in DMSO (163 μL; 315 nmol; 8.5 eq.) and reaction buffer (18 μL) were added to the trastuzumab solution, resulting in a final concentration of 15% (v/v) DMSO with a final antibody concentration of 5.0 mg/mL. The reaction was incubated at 22° C. for 1.5 h.

After 1.5 h at 22° C., the reaction mixture was purified by preparative SEC on a HiLoad 16/600 Superdex 200 pg column equilibrated with PBS, pH 7.2 containing 10% (v/v) glycerol. The flow rate was kept constant at 1.5 mL/min. Fractions were collected and analysed by analytical HIC and analytical SEC. Fractions containing monomeric ADC without free (6) reagent and displaying average DARs between 8-32 were pooled and concentrated to 3.0 mg/mL using Vivaspin 20 centrifugal concentrators (PES membrane, 30 kDa MWCO) equilibrated with PBS, pH 7.2 containing 10% (v/v) glycerol. Concentrated conjugate sample was sterile filtered through a 0.22 μm pore size, PVDF membrane filter.

A preliminary characterisation of the MMAE ADC was carried out by HIC, SEC, and quantified by UV and endotoxin levels were determined (analytical results shown in Table 1a). The ADC was not observed to undergo aggregation within the storage buffer solution at a concentration of 3.0 mg/mL, despite having a high average DAR of 15. Further, preliminary studies suggest that the ADC has an improved serum stability compared to a control ADC.

The HIC experiments were repeated and reveal that the average DAR of the MMAE ADC is 17.1 (see Table 1b).

TABLE 1a
Analytical summary of ADC from preliminary experiment

| Analysis | Results |
|---|---|
| DAR variants (HIC) | DAR 0: 0.6% |
| | DAR 4: 0.7% |
| | DAR 8: 27.4% |
| | DAR 12: 9.2% |
| | DAR 16: 27.6% |
| | DAR 20: 25.3% |
| | DAR 24: 9.2% |
| | Average DAR: 15 |
| % Purity (SEC) | >99% monomeric |
| Concentration-UV | 3.0 mg/mL |
| Amount (by UV Analysis) | 5.1 mg |
| Endotoxin (EU/mg) | 0.12 |

TABLE 1b
Repeat analytical HIC experiments on ADC

| Analysis | Results |
|---|---|
| DAR variants (HIC) | DAR 0: 0.6% |
| | DAR 4: 0.7% |
| | DAR 8: 27.4% |
| | DAR 16: 36.8% |
| | DAR 24: 25.3% |
| | DAR 32: 9.2% |
| | Average DAR: 17.1 |

### Example 4

### Cell Viability Assay with MMAE ADC

The CellTiter-Glo® luminescence viability assay was used to measure the inhibitory effect of the MMAE ADC prepared in Example 3 on cell growth. Any reduction in cell proliferation or metabolic activity is indicative of the cytotoxic and/or cytostatic properties of a compound.

Her2^{High} SK-BR-3 (human breast adenocarcinoma, ATCC® HTB-30, Manassas, Va., United States) were cultured in McCoy's 5A medium supplemented with 200 U/mL penicillin, 200m/mL streptomycin and 20% heat-inactivated fetal bovine serum. Her2^{Low} JIMT-1 (human breast carcinoma, ACC589, DSMZ, Braunschweig, Germany) were cultured in DMEM GlutaMax® medium supplemented with 200 U/mL penicillin, 200 μg/mL streptomycin and 10% heat-inactivated fetal bovine serum. Her2^{Negative} NCI-H520 (human lung squamous cell carcinoma, ATCC-HTB-182) were cultured in RPMI medium supplemented with 200 U/mL penicillin, 200 μg/mL streptomycin and 10% heat-inactivated fetal bovine serum.

SK-BR-3, JIMT-1 and NCI-H520 cells were seeded in 96-well plates at a density of 5×10³, 2×10³ and 2.5×10³ cells in 100 μL growth medium, respectively, and incubated for 24 hours at 37° C./5% CO₂. After 24 hours, growth medium was replaced with serial dilutions of test samples (ADC, Kadcyla® and free payload MMAE) in growth medium.

After 96 hours in the presence of ADCs or controls, viability was detected using the CellTiterGlo® luminescence assay. Assay plates were equilibrated at room temperature for 20 minutes before addition of 100 μL CellTiter-Glo® reagent per well. The plates were then mixed for 3 minutes at 300 rpm to assist cell lysis and incubated for a further 20 minutes at room temperature to stabilise the luminescence signal. Luminescence was recorded using a SpectraMax i3x plate reader with a default integration time of 0.5 s/well.

Data were then analysed using a four-parameter non-linear regression model. Viability was expressed as a percentage of untreated cells, 100% viability corresponding to the average luminescence of wells containing cells treated with complete medium only. The percentage viability (Y-axis) was plotted against the drug concentration in nM (X-axis) and the software was used to calculate the IC₅₀ values for all tested compounds.

A strong anti-proliferative effect was observed with both SK-BR-3 (Her2^{High}) and JIMT-1 (Her2^{Low}) cell lines for the ADC (Table 2). Minimal anti-proliferative effect was observed with NCI-H520 (Her2^{Negative}) cell line.

TABLE 2
Summary of the anti-proliferative effect (IC₅₀ values) of ADC
in comparison to Kadcyla ® and free payload MMAE
on SK-BR-3, JIMT-1 and NCI-H520 cells (n = 3).

| | IC₅₀ (nM) | | |
|---|---|---|---|
| Sample | SK-BR-3 | JIMT-1 | NCI-H520 |
| ADC | 0.008 ± 0 | 0.228 ± 0.099 | >100 |
| Kadcyla ® | 0.047 ± 0.021 | 5.921 ± 4.313 | 37.307 ± 16.075 |
| MMAE | 0.284 ± 0.138 | 0.126 ± 0.053 | 0.334 ± 0.114 |
| Trastuzumab | 0.141 ± 0.138 | >1000 | >1000 |

### Example 5

### In Vivo Efficacy Study of MMAE ADC

The objective of this study was to evaluate the in vivo anti-tumour efficacy of the MMAE ADC of Example 3 in the subcutaneous NCI-N87 human gastric cancer CDX model in female BALB/c Nude mice.

Experimental Design

TABLE 3
Description of experimental design for efficacy study

| | | | Dosing | | |
|---|---|---|---|---|---|
| | | Dose | Volume | Dosing | |
| N¹ | Group Treatment | (mg/kg) | (mL/kg)² | Route | Schedule³ |
| 10 | Vehicle | — | 5 | IV | Single dose |
| 10 | T-DM1 (Kadcyla) | 3 | 5 | IV | Single dose |
| 10 | ADC | 4 | 5 | IV | Single dose |
| 10 | ADC | 1.33 | 5 | IV | Single dose |
| 10 | ADC | 0.33 | 5 | IV | Single dose |

Note:
¹N: animal number
²Dosing volume: adjust dosing volume based on body weight to 5 mL/kg
³The experiment duration was 42 days

Experimental Methods and Procedures

Animals

Species: Mus musculus

Strain: BALB/c Nude

Age: 6-8 weeks

Sex: female

Body weight: 18-22 g

Cell Culture

The NCI-N87 tumor cells (ATCC, Manassas, Va., cat #CRL-5822) were maintained in vitro as a monolayer culture in RPMI-1640 medium supplemented with 10% fetal bovine serum, 1% Antibiotic-Antimycotic, at 37° C. in an atmosphere of 5% CO₂ in air. The tumour cells were routinely subcultured twice weekly by trypsin-EDTA treatment. The cells growing in an exponential growth phase were harvested and counted for tumour inoculation.

Tumour Inoculation and Animal Grouping

Each mouse for efficacy study was inoculated subcutaneously at the right flank with NCI-N87 tumour cells (10×10⁶) in 0.2 mL of PBS supplemented with Matrigel (1:1) for tumour development. Treatments were started on day 6 after tumour inoculation when the average tumour size reached approximately 198 mm³. The animals were assigned into groups using an Excel-based randomization software performing stratified randomization based upon their tumor volumes. Each group consisted of 10 tumour-bearing mice. The testing article was administrated to the mice according to the predetermined regimen.

Observations

All the procedures related to animal handling, care and the treatment in the study were performed according to the guidelines approved by the Institutional Animal Care and Use Committee (IACUC) of the CRO following the guidance of the Association for Assessment and Accreditation of Laboratory Animal Care (AAALAC). At the time of routine monitoring, the animals were daily checked for any effects of tumour growth and treatments on normal behaviour such as mobility, food and water consumption (by looking only), body weight gain/loss (body weights were measured twice weekly), eye/hair matting and any other abnormal effect as stated in the protocol. Death and observed clinical signs were recorded on the basis of the numbers of animals within each subset.

Tumour Measurements and Endpoints

The major endpoint was to see if the tumour growth could be delayed or mice could be cured. Tumour size was measured twice weekly in two dimensions using a calliper, and the volume was expressed in mm³ using the formula: V=0.5 a×b² where a and b are the long and short diameters of the tumour, respectively. The tumour sizes were then used for the calculations of both T/C and TGI values.

The T/C (%) value is calculated for each group using the formula: T/C (%)=T_{RTV}/C_{RTV}×100% (T_{RTV}: relative tumour volume (RTV) of the treatment group; C_{RTV}: relative tumour volume (RTV) of the vehicle control group on the same day with T_{RTV}). The relative tumour volume (RTV) is calculated for each group using the formula: RTV=Vₜ/V₀; V₀ is the average tumour volume on the first day of treatment, Vₜ is the average tumour volume on a given day.

TGI was calculated for each group using the formula: TGI (%)=[1−(Tᵢ−T₀)/(Vᵢ−V₀)]×100 (Tᵢ is the average tumour volume of a treatment group on a given day, T₀ is the average tumour volume of the treatment group on day 0, Vᵢ is the average tumour volume of the vehicle control group on the same day with Tᵢ, and V₀ is the average tumour volume of the vehicle group on the first day of treatment.

Statistical Analysis

Summary statistics, including mean and the standard error of the mean (SEM), are provided for the tumour volume of each group at each time point. Statistical analysis of difference in the tumour volume among the groups were conducted on the data obtained the 42^{nd} day post treatment start. A one-way ANOVA was performed to compare the tumour volume among groups, and a significant F -statistics was obtained, comparisons between groups were carried out with Games-Howell test. All data were analysed using SPSS 17.0. p<0.05 was considered to be statistically significant.

Results

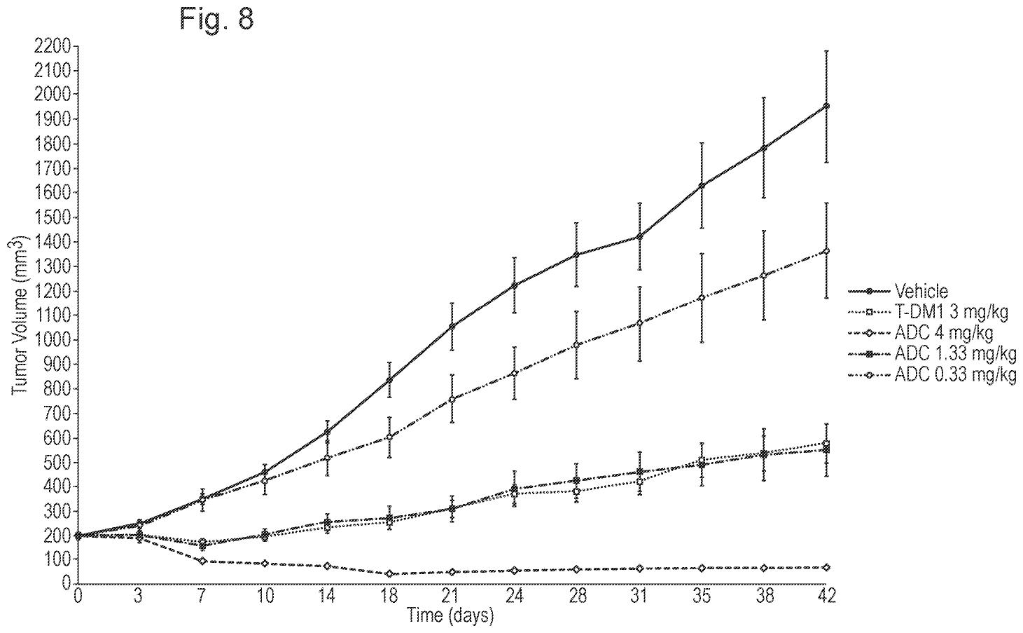

In this study, the therapeutic efficacy of the MMAE ADC in the treatment of the NCI-N87 human gastric cancer CDX model was evaluated. The results of tumour volumes are shown in FIG. 8. ADC significantly inhibited NCI-N87 tumour growth. Especially, ADC at 4 mg/kg (T/C=3.44%, TGI=107.47%; p<0.001) led to tumour regression with the average tumour volume of 67 mm³ on PG-D42. In addition, the anti-tumour activity of the ADC is shown to be dose-dependent. The positive control article T-DM1 at 3 mg/kg (T/C=29.37%, TGI=78.58%; p=0.004) also produced significant anti-tumor activity with a mean tumour volume of 574 mm³ on PG-D42, similar to the activity shown by the novel MMAE ADC at 1.33 mg/kg (T/C=27.17%, TGI=80.32%; p=0.003). The MMAE ADC was tolerated well by the tumour-bearing mice.

In summary, the novel ADC produced significant anti-tumour activity against the NCI-N87 human gastric cancer CDX model and was well tolerated by the tumour-bearing animals in this study.

### Example 6

### Preparation of Polymer (7)

A target polymer of formula (7) (Scheme 6) was synthesised via the following synthetic steps. The polymer (7) was built from Boc-Ser(-tBu)-DAP(-Fmoc)-OH dipeptide (7a) and Fmoc-N-amido-PEG-acid building blocks using Solid Phase Synthesis (SPS) to enable construction of a polymer of a specific number of units. The polymer can then be cleaved from the resin to afford the product as a monodisperse polymer.

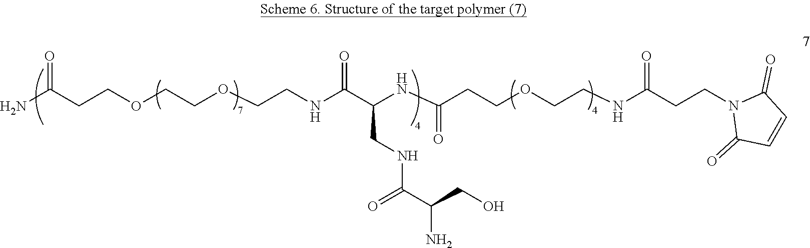

The Fmoc-N-amido-PEG-acid building blocks were purchased from a commercial supplier and Boc-Ser(-tBu)-DAP(-Fmoc)-OH dipeptide (7a) was synthesised as described below. After building the polymer using SPS, the terminal amine group was capped by coupling with 3-maleimidopropionic acid, followed by a single cleavage and deprotection step using a cocktail of trifluoroacetic acid (TFA), triisopropylsilane (TIS) and water to release the polymer (7).

Step a: Preparation of Boc-Ser(-tBu)-DAP(-Fmoc)-OH Dipeptide (7a)

To a solution of Boc-Ser(OtBu)-OH (3.71 g, 14.17 mmol) and NHS (3.26 g, 28.35 mmol) in DCM (150 mL) was added DCC (2.92 g, 14.17 mmol). The reaction mixture was stirred for 2 h at room temperature. The mixture was then filtered through a frit, the solid was rinsed with a small volume of DCM and the filtrate was concentrated under vacuum to give an amber viscous oil. The oil was dissolved in THF (50 mL) and the solution was added to a suspension of Fmoc-DAP-OH (3.7 g, 10.32 mmol) and NaHCO₃ (0.87 g, 10.32 mmol) in a mixture H₂O:THF (1:1, 180 mL total volume). The resulting reaction mixture was stirred for 16 h at room temperature. THF was removed under vacuum and the mixture was then acidified to pH˜3 with dilute HCl. The aqueous layer was extracted with EtOAc (3×100 mL) and the combined organic layers were dried over Na₂SO₄, filtered, and concentrated. The crude oil residue was purified by silica gel (120 g) column chromatography using a gradient method of 0-10% MeOH in DCM. The fractions containing the product were combined and concentrated under vacuum to afford compound (7a) (2.6 g, 40%) as a white solid. A portion of the crude material (1.8 g) was further purified and was loaded onto an C18 column and eluted with a mobile phase of 5-70% MeCN in H₂O (+0.05% formic acid). The fractions containing pure product were combined, partially concentrated, and lyophilised to afford 1.2 g of compound, 66% yield, as a fluffy white powder.

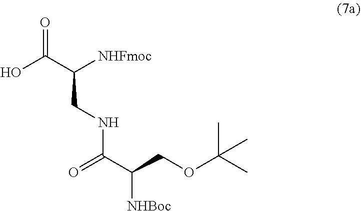

Step b: Synthesis of Polymer (7) by SPS

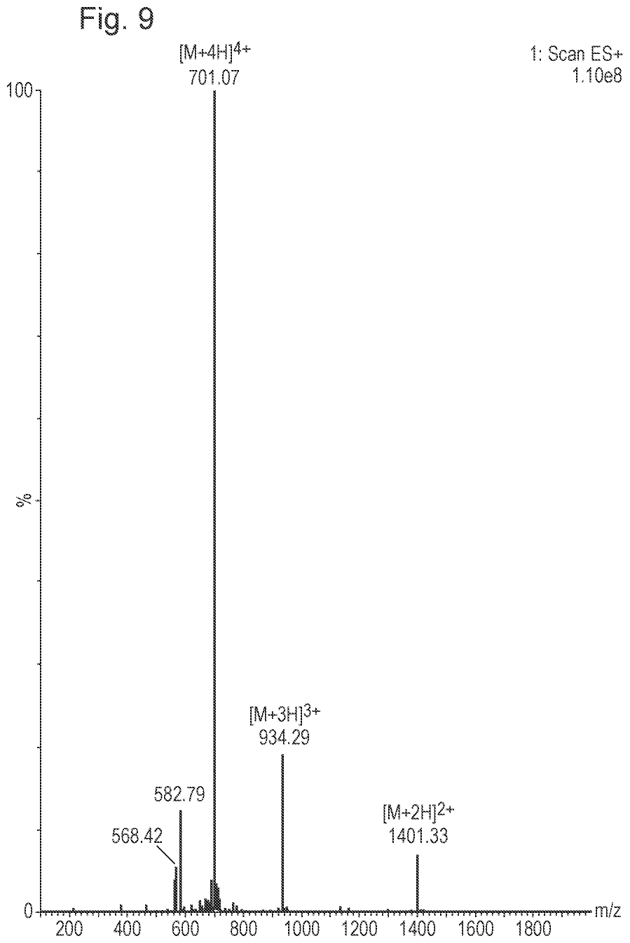

The SPPS of polymer (7) involved four cycles of deprotection and coupling, each cycle comprising i) Fmoc deprotection, ii) coupling of Fmoc-N-amido-PEG8-acid, iii) Fmoc deprotection, iv) coupling of Boc-Ser(-tBu)-DAP(-Fmoc)-OH dipeptide (7a) (for the first 4 cycles). An additional final cycle comprised v) Fmoc deprotection, vi) coupling of Fmoc-N-amido-PEG4-acid, vii) Fmoc deprotection and viii) 3-maleimidopropionic acid coupling. Finally, the polymer was cleaved from the resin. The final sample of polymer (7) was prepared after precipitation of the crude material in cold diethyl ether. The material was dried overnight by lyophilisation. 368 mg crude polymer (7) was isolated, 66% yield (LC-MS characterization in FIG. 9).

### Example 7

### Preparation of Polymer (8)

A target polymer of formula (8) (Scheme 7) was synthesised via the following synthetic steps. The polymer (8) was built from Boc-Ser(-tBu)-DAP(-Fmoc)-OH dipeptide (7a) prepared in Example 6 and Fmoc-N-amido-PEG-acid building blocks using Solid Phase Synthesis (SPS) to enable construction of a polymer of a specific number of units. The polymer can then be cleaved from the resin to afford the product as a monodisperse polymer.

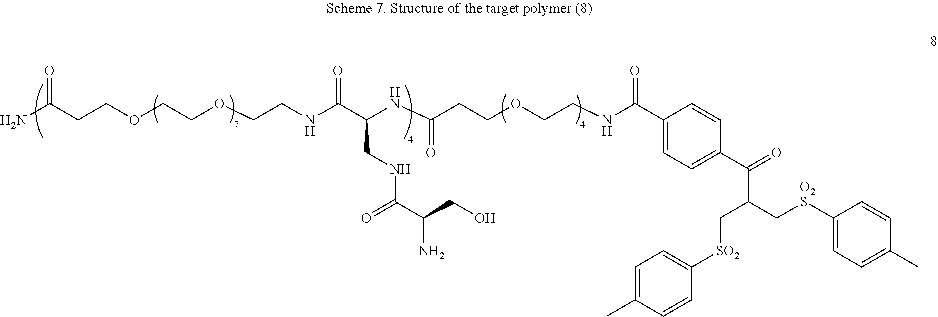

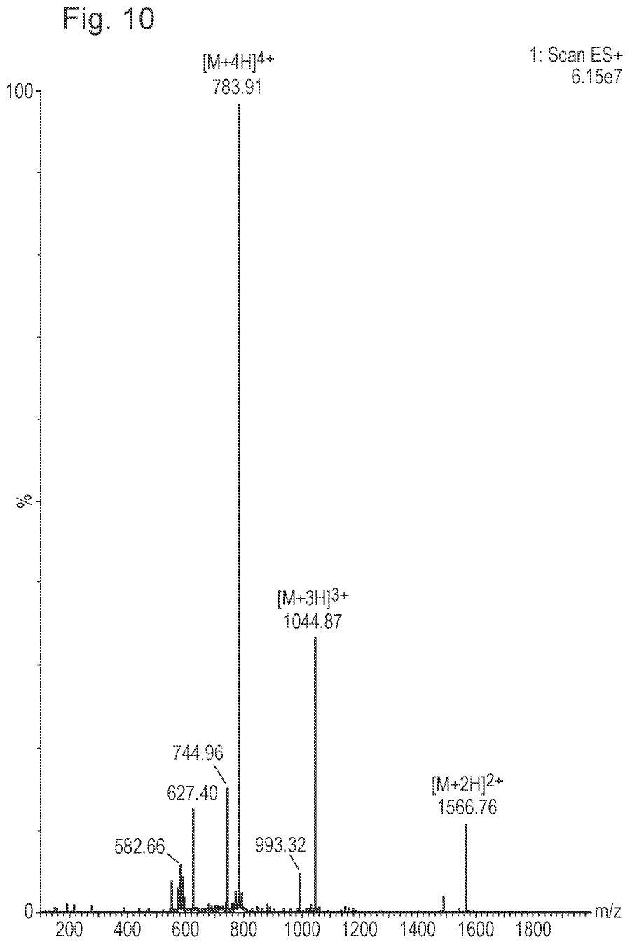

The synthesis of polymer (8) was executed with 1.4 g of ProTide Rink Amide LL Resin following the procedure used for the synthesis of polymer (7) except for the last step viii), which involved coupling with ThioBridge® HOBt ester instead of 3-maleimidopropionic acid, followed by resin cleavage and deprotection of the t-Bu and Boc groups. ThioBridge® HOBt ester was prepared as described in WO2016/063006, pages 25-26. Due to possible elimination of the tosyl group of the ThioBridge® moiety, 4-methylmorpholine was used as base. The resin cleavage/deprotection of polymer (8) was done by using neat TFA (20 mL) in 2 h at room temperature. The TFA was separated, the resin was washed with TFA (10 mL) for 10 min. Combined TFA mother liquor was concentrated to 2-3 mL. The final product was prepared after precipitation of the crude material in cold diethyl ether (100 mL). The material was dried overnight by lyophilisation. 505 mg of polymer (8) was isolated in 60.6% yield (LC-MS characterization in FIG. 10).

### Example 8

### Preparation of SN-38 Drug Payload Reagent (11)

Step a: Synthesis of Polymer (9)

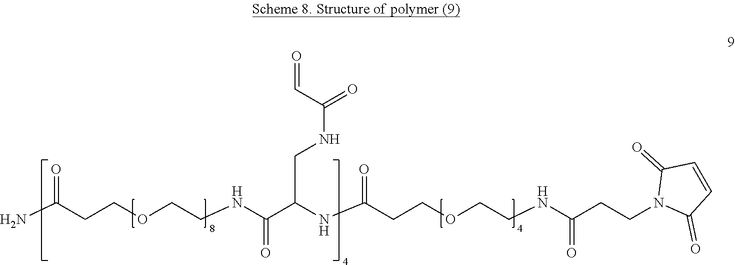

Oxidation with sodium periodate was performed on the crude polymer (7) to achieve the synthesis of polymer (9) (Scheme 8).

To a solution of the crude polymer (7) (42 mg, 15 μmol) in mixture of DPBS (1000 mL) and acetonitrile (100 mL) was added NaIO₄ (80 mg, 375 μmol) as a solid in one portion. The reaction mixture was occasionally shaken over a period of 1 hour at ambient temperature. The reaction mixture was filtered by 0.45 μm PTFE and purified immediately by prep-HPLC (C18) using a gradient of 5-55% MeCN in H₂O (0.05% Formic acid) over 45 min.

Fractions were analysed by LC-MS and RP-UPLC. The fractions containing the desired product were combined. MS (ESI), m/z: [M+2H]²⁺ calculated: 1339.2, observed: 1338.69; [M+3H]³⁺ calculated: 893.1, observed: 893.07; [M+4H]⁴⁺ calculated: 670.1, observed: 670.05.

Step b: Synthesis of SN-38 Reagent (10)

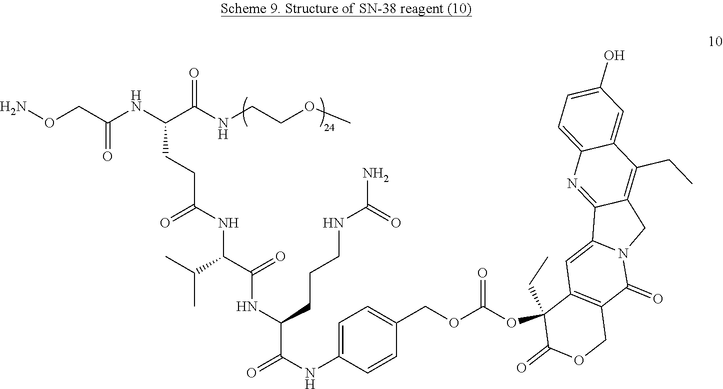

The synthesis of SN-38 reagent (10) (Scheme 9) was achieved via the following steps.

### 1. Preparation of Boc-SN-38

To a suspension of SN-38 (1 g, 2.55 mmol) in anhydrous DCM (80 mL) was added (Boc)₂O (723 mg, 3.31 mmol) and anhydrous pyridine (6.05 mL, 7.65 mmol). The reaction mixture was stirred at room temperature for 24 hours under argon atmosphere. The reaction mixture was washed with 0.5N HCl solution (3×35 mL) followed by saturated NaHCO₃ solution (1×50 mL) and brine (50 mL). The organic layer was dried over Mg₂SO₄, filtered and concentrated to dryness to concentrated under vacuum to afford pure Boc-SN-38 (1.23 g, 98%) as a yellow solid. MS (ESI), m/z: [M+H]⁺ calculated: 493.19, observed: 493.25.

### 2. Preparation of Fmoc-Val-Cit-PAB-(Boc-SN-38)

To a suspension of Boc-SN-38 (0.754 g, 1.53 mmol) in anhydrous DCM (15 mL) was added DMAP (187 mg, 1.53 mmol) and DIPEA (1.34 mL, 7.67 mmol). The reaction mixture was placed in ice-bath. Triphosgene (195 mg, 0.66 mmol) was added dropwise as a solution in DCM (4 mL). The reaction mixture was stirred in ice-bath for 5 min and then 10 min at ambient temperature. Fmoc-Val-Cit-PAB (830 mg, 1.38 mmol) was dissolved in a mixture of DMSO (5 mL) and DCM (5 mL) and the solution was added to the reaction mixture. The resulting mixture was stirred at room temperature for 2 hours. The reaction mixture was concentrated and diluted with EtOAc (400 mL). The organics were washed with 5% aq. NaHCO₃ (2×40 mL), brine (40 mL), dried over Na₂SO₄ and concentrated. The crude residue was purified by silica gel column chromatography using DCM-MeOH gradient method (0-5%) to afford Fmoc-Val-Cit-PAB-(Boc-SN-38) (1.2 g, 77%) as a yellow solid. MS (ESI), m/z: [M+H]⁺ calculated: 1020.46, observed: 1020.1.

### 3. Preparation of H—Val-Cit-PAB-SN-38

To a solution of Fmoc-Val-Cit-PAB-(Boc-SN-38) (1.2 g, 1.07 mmol) in anhydrous DMF (10 mL) was added piperidine (1.06 mL, 10.7 mmol). The reaction mixture was stirred at room temperature for 2.5 hours. The reaction mixture was concentrated to dryness and a mixture Et₂O/EtOH 10/1 by v/v/ (50 mL) was added. The formed solid was separated by centrifugation and washed by Et₂O (2×40 mL) to afford val-cit-PAB-SN-38 (0.641 mg, 75%) as a yellow solid, which was used in the next step without purification. MS (ESI), m/z: [M+H]+ calculated: 798.34, observed: 797.89.

### 4. Preparation of Fmoc-Glu(OH)-PEG24u

To a mixture of mPEG24-NH₂ (1.53 g, 1.4 mmol) and Fmoc-Glu(t-OBu)OH (0.57 g, 1.34 mmol) in DMF (10 mL) was added NMM (444 ml, 4.05 mmol). The mixture was cooled in an ice-bath. HATU (0.641 g, 1.69 mmol) was added to the flask as solid portionwise. The reaction mixture was stirred at room temperature for 16 hours. The mixture was concentrated in vacuum. The residue was purified by reverse phase column chromatography using Buffer A: 100% H₂O, 0.1% Formic acid and Buffer B: 100% Acetonitrile, 0.1% Formic acid gradient method (0-60%). After lyophilization of pooled fractions, the solid was treated with a mixture of TFA (5 mL) and DCM (10 mL) at room temperature for 3 hours. The mixture was concentrated, and the residue was purified by reverse phase column chromatography using Buffer A: 100% H₂O, 0.05% TFA and Buffer B: 100% Acetonitrile, 0.05% TFA gradient method (0-60%). Pooled fractions were lyophilized to afford pure Fmoc-Glu(OH)-PEG24u (1.44 g, 74.6%) as a white solid. MS (ESI), m/z: [M+H]⁺ calculated: 1439.8, observed: 1439.47.

### 5. Preparation of Fmoc-Glu(Val-Cit-PAB-SN-38)-PEG24u

To a mixture of Fmoc-Glu(OH)-mPEG24u (0.35 g, 0.243 mmol) and Val-Cit-PAB-SN-38 (0.21 g, 0.267 mmol) in DMF (4 mL) was added NMM (80 ml, 0.729 mmol). The mixture was cooled in an ice-bath. HATU (0.115 g, 0.304 mmol) was added to the flask as solid portionwise. The reaction mixture was stirred at room temperature for 1 hour. The mixture was concentrated in vacuum. The residue was purified by reverse phase column chromatography using Buffer A: 100% H₂O, 0.1% Formic acid and Buffer B: 100% Acetonitrile, 0.1% Formic acid gradient method (0-60%). Pooled fractions were lyophilized to afford pure Fmoc-Glu(Val-Cit-PAB-SN-38)-PEG24u (0.45 g, 83.3%) as an off-white solid. MS (ESI), m/z: [M+2H]²⁺ calculated: 1110.06, observed: 1110.13.

### 6. Preparation of Glu(Val-Cit-PAB-SN-38)-PEG24u

To a solution of Fmoc-Glu(Val-Cit-PAB-SN-38)-PEG24u (0.427 g, 0.192 mmol) in anhydrous DMF (2.5 mL) was added piperidine (285 ml, 2.8 mmol). The reaction mixture was stirred at room temperature for 1 hour. The reaction mixture was concentrated to dryness and Et₂O (50 mL) was added. The formed solid was separated by centrifugation and washed by Et₂O (2×40 mL) to afford pure Glu(Val-Cit-PAB-SN-38)-PEG24u (340 mg, 87.2%) as a yellow solid. MS (ESI), m/z: [M+2H]²⁺ calculated: 999.02, observed: 999.10.

### 7. Preparation of (Boc)₂N—OCH₂CO-Glu(Val-Cit-PAB-SN-38)-PEG24u

To a mixture of H-Glu(Val-Cit-PAB-SN-38)-PEG24u (335 mg, 0.167 mmol) and Boc₂N—OCH₂COOH (54 mg, 0.184 mmol) in DMF (4 mL) was added NMM (61 ml, 0.553 mmol). The mixture was cooled in an ice-bath. HBTU (80 mg, 0.210 mmol) was added as solid portionwise. The reaction mixture was stirred at room temperature for 1.5 hour. The mixture was concentrated in vacuum. The residue was purified by reverse phase column chromatography using Buffer A: 100% H₂O, 0.1% Formic acid and Buffer B: 100% Acetonitrile, 0.1% Formic acid gradient method (0-75%). Pooled fractions were lyophilized to afford pure (Boc)₂N—OCH₂CO-Glu(Val-Cit-PAB-SN-38)-PEG24u (0.320 g, 83.3%) as an off-white solid. MS (ESI), m/z: [M+2H]²⁺ calculated: 1135.58, observed: 1135.15.

### 8. Preparation of H₂N—OCH₂CO-Glu(Val-Cit-PAB-SN-38)-PEG24u—SN-38 Reagent (10)

Neat formic acid (25 mL) was added to (Boc)₂N—OCH₂CO-Glu(Val-Cit-PAB-SN-38)-PEG24u solid (315 mg, 0.138 mmol) and the solution was stirred at room temperature for 2 hours. The mixture was concentrated in vacuum. The residue was purified by reverse phase column chromatography using Buffer A: 100% H₂O, 0.1% Formic acid and Buffer B: 100% Acetonitrile, 0.1% Formic acid gradient method (0-65%). Pooled fractions were lyophilized to afford pure H₂N—OCH₂CO-Glu(Val-Cit-PAB-SN-38)-PEG24u formic acid salt (10) (0.165 g, 56.3%) as a yellow solid. MS (ESI), m/z: [M+2H]²⁺ calculated: 1035.53, observed: 1035.57.

Step c: SN-38 Reagent (10) Coupling to Polymer (9) to Generate SN-38 Reagent (11)

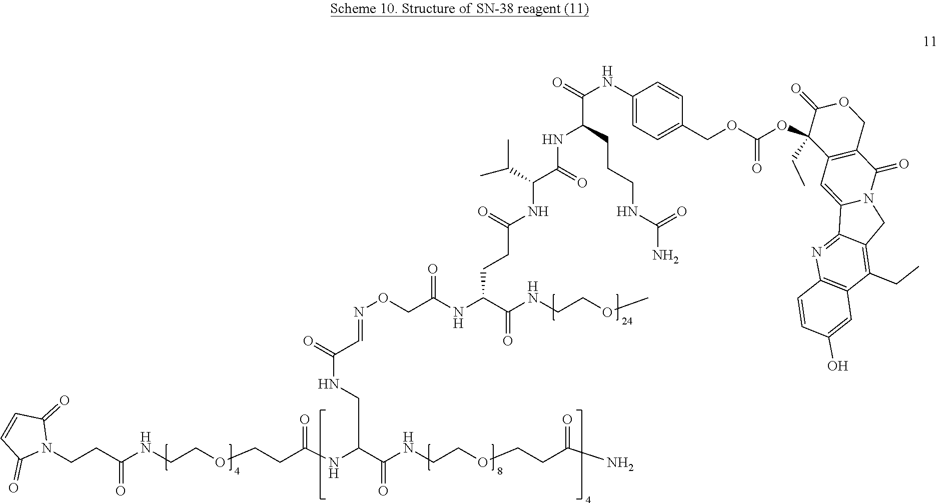

Oxime ligation was performed between the purified aldehyde-functionalised polymer (9) and SN-38 reagent (10) to generate conjugate bearing 4 copies of drug payload SN-38 (11) (Scheme 10).

H₂N—OCH₂CO-Glu(Val-Cit-PAB-SN-38)-PEG24u formate (10) (87 mg, 41 μmol) was dissolved in a mixture of MeCN:H₂O with 0.05% formic acid, 1:1 v/v (250 μL) and added to the combined HPLC fractions containing aldehyde-functionalised polymer (9). The resulting mixture was stirred at room temperature for 1.5 hours. Full conversion of the aldehyde polymer was observed by RP-UPLC analysis; the desired product formation was confirmed by LC-MS. The reaction mixture was concentrated in vacuum and residue was directly purified by preparative RP-HPLC (C18) using a gradient of 20-70% MeCN in H₂O (0.05% formic acid) over 45 min.

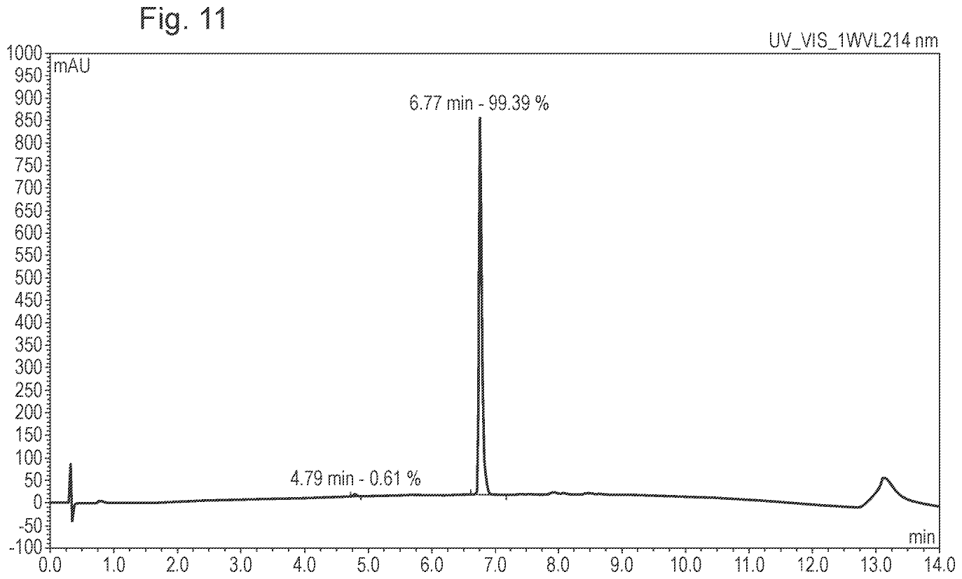

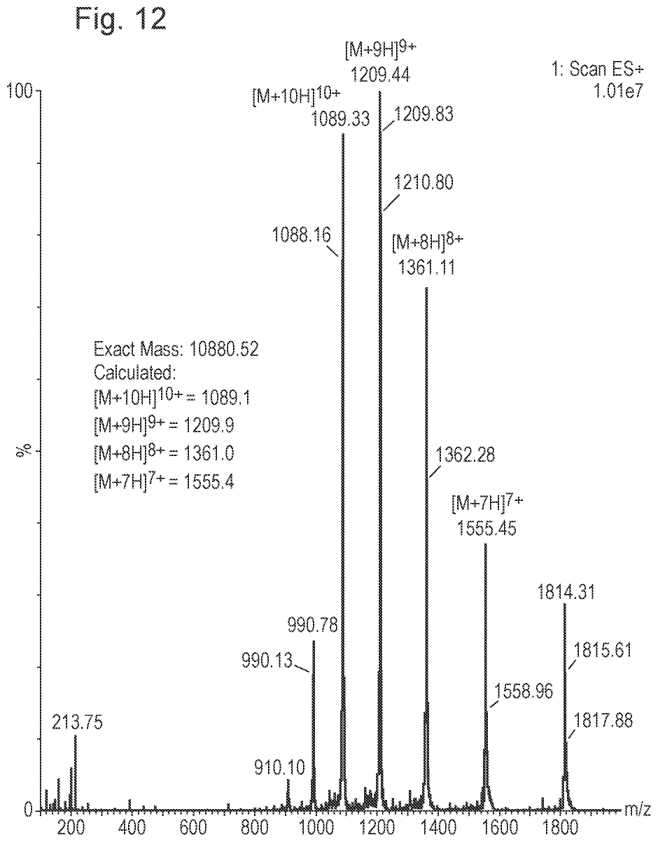

Fractions were analysed by LC-MS and RP-UPLC (FIGS. 11 and 12). The fractions containing the desired product were combined and lyophilized to give desired SN-38 reagent (11) (31 mg, 19%) as a white solid. MS (ESI), m/z: [M+10H]¹⁰⁺ calculated: 1089.1, observed: 1089.33; [M+9H]⁹⁺ calculated: 1209.9, observed: 1209.44; [M+8H]⁸⁺ calculated: 1361.0; observed: 1361.11; [M+7H]⁷⁺ calculated: 1555.4; observed: 1555.45.

### Example 9

### SN-38 Reagent (11) ADC Preparation by Conjugation of SN-38 Reagent (11) to Trastuzumab

Trastuzumab at 10.49 mg/mL in DPBS, pH 7.2, 5 mM EDTA (2.097 mL; 22.0 mg; 151 nmol; 1.0 eq.) was diluted with Dulbecco's PBS, pH 7.2, 5 mM EDTA, (2.233 mL). A 5 mM solution of TCEP in endotoxin-free water (69.3 μL; 347 nmol; 2.3 eq.) was added to the dilute trastuzumab solution. The reduction was allowed to proceed at 40° C. for 1.5 h with a final antibody concentration of 5.0 mg/mL.

After 1.5 h at 40° C., the reduction mixture was diluted with Dulbecco's PBS, pH 7.2, 5 mM EDTA (550 μL), and allowed to cool down to 22° C. A 17.90 mg/mL (1.64 mM) solution of SN-38 reagent (11) in 1:1 MeCN/water was prepared by dissolving 10.0 mg (919 nmol) of SN-38 reagent (11) (MW=10887 g.mol⁻¹) into 559 μL of a 1:1 mixture of MeCN/water. SN-38 reagent (11) solution in 1:1 MeCN/water (550 μL; 9.84 mg; 906 nmol; 6.0 eq.) was added to the reduced trastuzumab solution, resulting in a final concentration of 5% MeCN and a final antibody concentration of 4.0 mg/mL. The conjugation reaction was allowed to proceed at 22° C. for 1 h. A further portion of SN-38 reagent (11) solution in 1:1 MeCN/water (68.75 μL; 1.23 mg; 113 nmol; 0.75 eq.) was added to the reduced trastuzumab solution and the conjugation reaction was allowed to proceed at 22° C. for 1 h.

After 2 h at 22° C., the reaction mixture was loaded onto a HiLoad 16/600 Superdex 200 pg column. Elution was carried out with DPBS, pH 7.2 buffer and a constant flow of 1.0 mL/min. Fractions with a monomeric purity>95% were pooled and sterile filtered through a 0.22 μm pore size, PVDF membrane filter. The final conjugate sample (40 mg; 18.0 mL) was obtained. The SN-38 reagent (11) ADC conjugate was characterised by HIC, SEC, LC-MS, SDS-PAGE and quantified by UV and endotoxin levels were determined (analytical results shown in Table 4).

TABLE 4
Analytical summary of SN-38 reagent (11) ADC

| Analysis | Results |
|---|---|
| DAR variants (HIC) | DAR 0: 1.4% |
| | DAR 4: 9.3% |
| | DAR 8: 34.4% |
| | DAR 16: 30.8% |
| | DAR 24: 15.7% |
| | DAR 32: 8.4% |
| | Average DAR: 15.2 |
| Average DAR (UV) | Average DAR: 17.9 |
| SDS-PAGE | Average DAR: 18.7 |
| % Purity (SEC) | 99.6% monomeric |
| Endotoxin (EU/mg) | 0.09 |
| Amount (by UV Analysis) | 40 mg |

### Example 10

### Preparation of SN-38 Drug Payload Reagent (13)

Step a: Synthesis of Polymer (9)

This was carried out as described in step (a) of Example 8.

Step b: Synthesis of SN-38 Reagent (12)

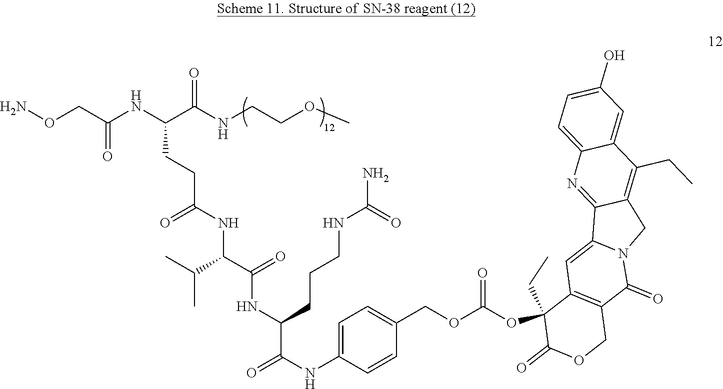

The synthesis of SN-38 reagent (12) (Scheme 11) was achieved as follows. Firstly, steps 1, 2 and 3 were carried out as described in Example 8. Then, the following steps were carried out.

### 4. Preparation of Fmoc-Glu(OH)-PEG12u

To a mixture of mPEG12—NH₂ (1.37 g, 2.44 mmol) and Fmoc-Glu(t-OBu)OH (1.012 g, 2.38 mmol) in DMF (10 mL) was added NMM (784 ml, 7.14 mmol). The mixture was cooled in an ice-bath. HATU (1.045 g, 2.75 mmol) was added to the flask as a solid portionwise. The reaction mixture was stirred at room temperature for 16 hours. The mixture was concentrated in vacuum. The residue was purified by reverse phase column chromatography using Buffer A: 100% H₂O, 0.1% Formic acid and Buffer B: 100% Acetonitrile, 0.1% Formic acid gradient method (0-65%). After lyophilization of pooled fractions, the solid was treated with a mixture of TFA (8 mL) and DCM (16 mL) at room temperature for 3 hours. The mixture was concentrated, and the residue was purified by reverse phase column chromatography using Buffer A: 100% H₂O, 0.05% TFA and Buffer B: 100% Acetonitrile, 0.05% TFA gradient method (0-65%). Pooled fractions were lyophilized to afford pure Fmoc-Glu(OH)-PEG12u (1.56 g, 72.1%) as a white solid. MS (ESI), m/z: [M+H]⁺ calculated: 911.47, observed: 911.5.

### 5. Preparation of Fmoc-Glu(Val-Cit-PAB-SN-38)-PEG12u

To a mixture of Fmoc-Glu(OH)-mPEG12u (0.311 g, 0.342 mmol) and H—Val-Cit-PAB-SN-38 (0.3 g, 0.376 mmol) in DMF (4 mL) was added NMM (124 ml, 1.13 mmol). The mixture was cooled in an ice-bath. HBTU (0.162 g, 1.130 mmol) was added to the flask as a solid portionwise. The reaction mixture was stirred at room temperature for 2 hours. The mixture was concentrated in vacuum. The residue was purified by reverse phase column chromatography using Buffer A: 100% H₂O, 0.1% Formic acid and Buffer B: 100% Acetonitrile, 0.1% Formic acid gradient method (0-60%). Pooled fractions were lyophilized to afford pure Fmoc-Glu(Val-Cit-PAB-SN-38)-PEG12u (0.410 g, 71.1%) as a yellow solid. MS (ESI), m/z: [M+H]⁺ calculated: 1690.8, observed: 1690.21.

### 6. Preparation of Glu(Val-Cit-PAB-SN-38)-PEG12u

To a solution of Fmoc-Glu(Val-Cit-PAB-SN-38)-PEG12u (0.4 g, 0.236 mmol) in anhydrous DMF (3 mL) was added piperidine (291 ml, 2.84 mmol). The reaction mixture was stirred at room temperature for 1 hour. The reaction mixture was concentrated to dryness and Et₂O (50 mL) was added. The formed solid was separated by centrifugation and washed by Et₂O (2×40 mL) to afford pure H-Glu(Val-Cit-PAB-SN-38)-PEG12u (321 mg, 92.7%) as a yellow solid. MS (ESI), m/z: [M+H]⁺ calculated: 1468.73, observed: 1468.39.

### 7. Preparation of (Boc)₂N—OCH₂CO-Glu(Val-Cit-PAB-SN-38)-PEG12u

To a mixture of H-Glu[Val-Cit-PAB-SN-38]-PEG(12u) (321 mg, 0.219 mmol) and Boc₂-N—OCH₂COOH (73 mg, 0.251 mmol) in DMF (4 mL) was added NMM (80 mL, 0.723 mmol). The mixture was cooled in an ice-bath. HBTU (104 mg, 0.274 mmol) was added as a solid portionwise. The reaction mixture was stirred at room temperature for 1.5 hours. The mixture was concentrated in vacuum. The residue was purified by reverse phase column chromatography using Buffer A: 100% H₂O, 0.1% Formic acid and Buffer B: 100% Acetonitrile, 0.1% Formic acid gradient method (0-75%). Pooled fractions were lyophilized to afford pure (Boc)₂N—OCH₂CO-Glu(Val-Cit-PAB-SN-38)-PEG12u (0.327 g, 85.9%) as a yellow solid. MS (ESI), m/z: [M+H]⁺ calculated: 1740.84, observed: 1741.54.

### 8. Preparation of H₂N—OCH₂CO-Glu(Val-Cit-PAB-SN-38)-PEG12u—SN-38 Reagent (12)

Neat formic acid (25 mL) was added to (Boc)₂N—OCH₂CO-Glu(Val-Cit-PAB-SN-38)-PEG12u solid (320 mg), and the solution was stirred at room temperature for 2 hours. The mixture was concentrated in vacuum. The residue was purified by reverse phase column chromatography using Buffer A: 100% H₂O, 0.1% Formic acid and Buffer B: 100% Acetonitrile, 0.1% Formic acid gradient method (0-65%). Pooled fractions were lyophilized to afford pure H₂N—OCH₂CO-Glu(Val-Cit-PAB-SN-38)-PEG12u formic acid salt (0.144 g, 50.1%) as a yellow solid. MS (ESI), m/z: [M+H]⁺ calculated: 1541.74, observed: 1541.87.

Step c: SN-38 Reagent (12) Coupling to Polymer (9) to Generate SN-38 Reagent (13)

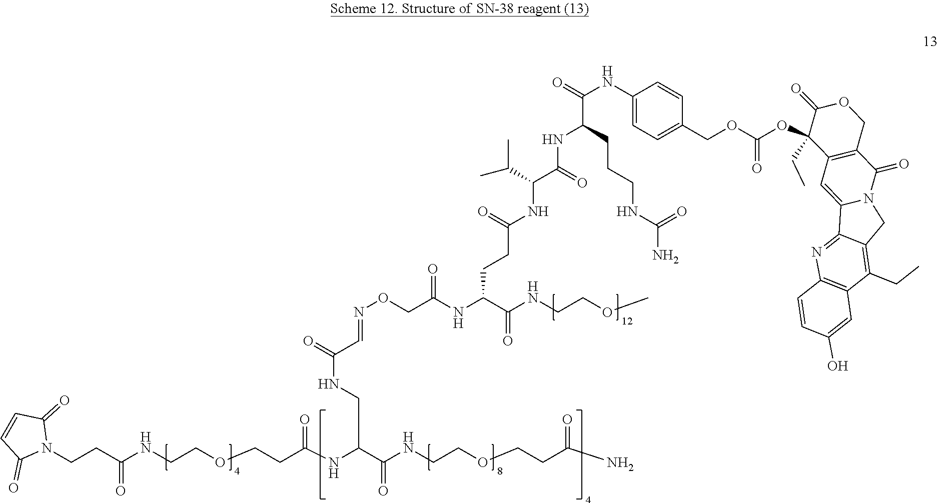

Oxime ligation was performed between the purified aldehyde-functionalised polymer (9) and SN-38 reagent (12) to generate conjugate bearing 4 copies of drug payload SN-38 (13) (Scheme 12).

H₂N—OCH₂CO-Glu(Val-Cit-PAB-SN-38)-PEG12u formate (12) (50 mg, 31 μmol) was dissolved in a mixture of MeCN:H₂O with 0.05% formic acid, 1:1 v/v (250 μL) and added to the combined HPLC fractions containing aldehyde-functionalised polymer (9). The resulting mixture was stirred at room temperature for 1.5 hours. Full conversion of the aldehyde polymer was observed by HPLC analysis; the desired product formation was confirmed by LC-MS. The reaction mixture was concentrated in vacuum and residue was directly purified by preparative RP-HPLC (C18) using a gradient of 20-70% MeCN in H₂O (0.05% formic acid) over 45 min.

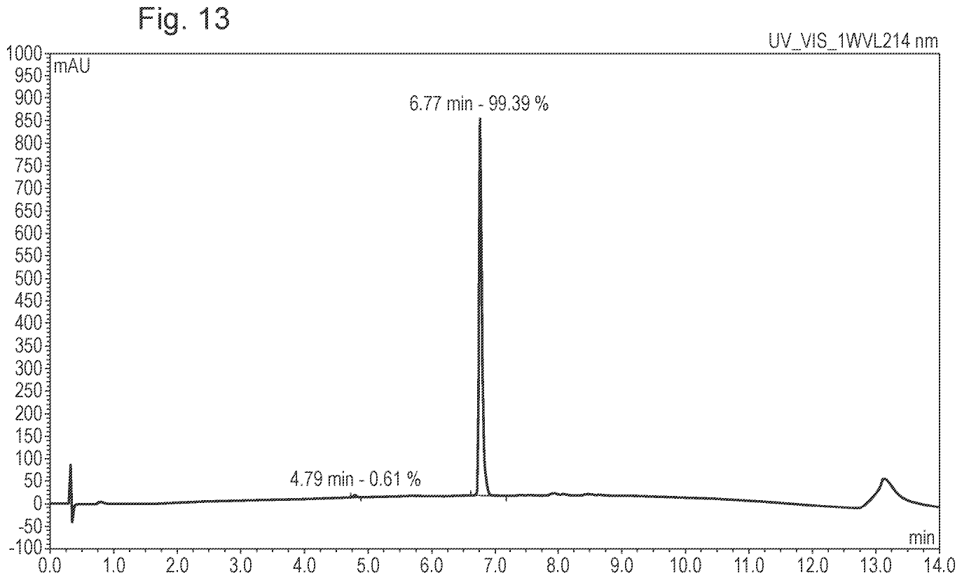

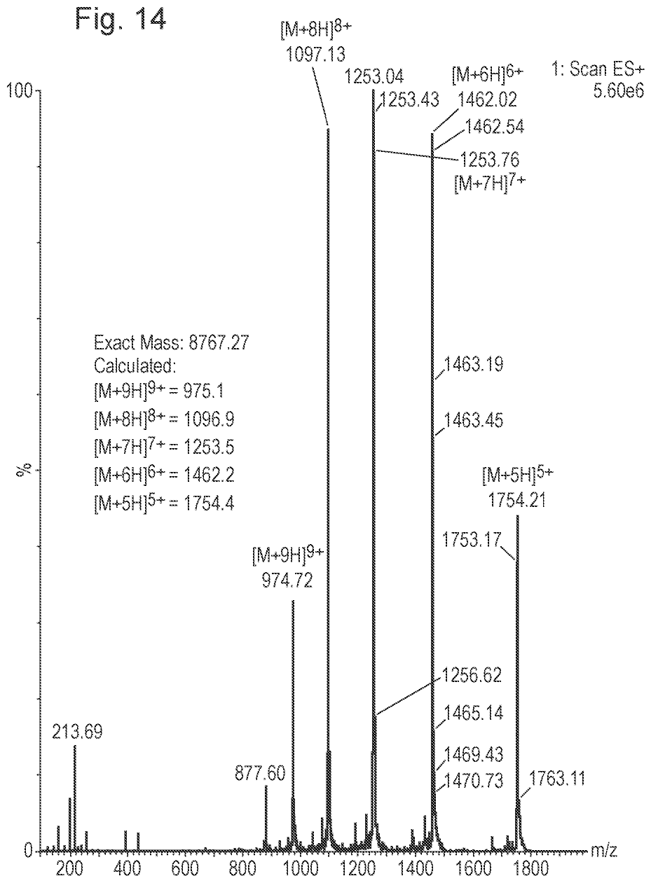

Fractions were analysed by LC-MS and HPLC (FIGS. 13 and 14). The fractions containing the desired product were combined and lyophilized to give desired SN-38 reagent (13) (30 mg, 19.5%) as a white solid.

MS (ESI), m/z: [M+9H]⁹⁺ calculated: 975.1, observed: 974.72; [M+8H]⁸⁺ calculated: 1096.9; observed: 1097.13; [M+7H]⁷⁺ calculated: 1253.5; observed: 1253.04; [M+6H]⁶⁺ calculated: 1462.2, observed: 1462.02; [M+5H]⁵⁺ calculated: 1754.4, observed: 1754.21.

### Example 11

### SN-38 Reagent (13) ADC Preparation by Conjugation of SN-38 Reagent (13) to Trastuzumab

Trastuzumab at 10.49 mg/mL in DPBS, pH 7.2, 5 mM EDTA (2.097 mL; 22.0 mg; 151 nmol; 1.0 eq.) was diluted with Dulbecco's PBS, pH 7.2, 5 mM EDTA, (2.233 mL). A 5 mM solution of TCEP in endotoxin-free water (69.3 μL; 347 nmol; 2.3 eq.) was added to the dilute trastuzumab solution. The reduction was allowed to proceed at 40° C. for 1.5 h with a final antibody concentration of 5.0 mg/mL. After 1.5 h at 40° C., the reduction mixture was diluted with Dulbecco's PBS, pH 7.2, 5 mM EDTA (550 μL), allowed to cool down to 22° C. A 14.4 mg/mL (1.64 mM) solution of SN-38 reagent (13) in 1:1 MeCN/water was prepared by dissolving 8.98 mg (1024 nmol) of SN-38 reagent (13) (MW=8772 g.mol⁻¹) into 623 μL of a 1:1 mixture of MeCN/water. SN-38 reagent (13) solution in 1:1 MeCN/water (550 μL; 7.93 mg; 906 nmol; 6.0 eq.) was added to the reduced trastuzumab solution, resulting in a final concentration of 5% MeCN and a final antibody concentration of 4.0 mg/mL. The conjugation reaction was allowed to proceed at 22° C. for 1 h.

After 1 h at 22° C., the reaction mixture was loaded onto a HiLoad 16/600 Superdex 200 pg column. Elution was carried out with DPBS, pH 7.2 buffer and a constant flow of 1.0 mL/min. The pooled fractions were purified again by preparative SEC to remove remaining reagent-related species. The material was loaded onto a HiLoad 16/600 Superdex 200 pg column. Elution was carried out with DPBS, pH 7.2+10% isopropanol buffer and a constant flow of 1.0 mL/min. Fractions with a monomeric purity>95% were pooled, buffer exchanged and concentrated by ultrafiltration/diafiltration using a Vivaspin 20 centrifugal concentrator (PES membrane, 30 kDa MWCO) into DPBS buffer. The final conjugate sample (25.8 mg; 7.0 mL) was sterile filtered through a 0.22 μm pore size, PVDF membrane filter.

The SN-38 reagent (13) ADC conjugate was characterised by HIC, SEC, LC-MS, SDS-PAGE and quantified by UV and endotoxin levels were determined (analytical results shown in Table 5).

TABLE 5
Analytical summary of SN-38 reagent (13) ADC

| Analysis | Results |
|---|---|
| DAR variants (HIC) | DAR 4: 10.1% |
| | DAR 8: 17.2% |
| | DAR 12: 19.6% |
| | DAR 16: 30.4% |
| | DAR 24: 16.3% |
| | DAR 32: 6.5% |
| | Average DAR: 15.2 |
| Average DAR (UV) | Average DAR: 20.3 |
| SDS-PAGE | Average DAR: 18.7 |
| % Purity (SEC) | 94.5% monomeric |
| Endotoxin (EU/mg) | 0.08 |
| Amount (by UV Analysis) | 25.8 mg |

### Example 12

### SN-38 Reagent (11) hIgG1 Isotype Control ADC Preparation by Conjugation of SN-38 Reagent (11) to an Irrelevant hIgG1 Isotype Control

Irrelevant hIgG1 at 7.82 mg/mL in DPBS, pH 7.2, 5 mM EDTA (1.023 mL; 8.0 mg; 55 nmol; 1.0 eq.) was diluted with Dulbecco's PBS, pH 7.2, 5 mM EDTA, (552 μL). A 5 mM solution of TCEP in endotoxin-free water (25.1 μL; 126 nmol; 2.3 eq.) was added to the dilute irrelevant hIgG1 solution. The reduction was allowed to proceed at 40° C. for 1 h with a final antibody concentration of 5.0 mg/mL.

After 1 h at 40° C., the reduction mixture was diluted with Dulbecco's PBS, pH 7.2, 5 mM EDTA (68.4 μL), allowed to cool down to 22° C. A 17.90 mg/mL (1.64 mM) solution of SN-38 reagent (11) in 1:1 MeCN/water was prepared by dissolving 10.0 mg (919 nmol) of SN-38 reagent (11) (MW=10887 g.mol⁻¹) into 559 μL of a 1:1 mixture of MeCN/water. SN-38 reagent (11) solution in 1:1 MeCN/water (332 μL; 5.94 mg; 546 nmol; 10.0 eq.) was added to the reduced irrelevant hIgG1 solution, resulting in a final concentration of 5% MeCN and a final antibody concentration of 4.0 mg/mL. The conjugation reaction was allowed to proceed at 22° C. for 1 h.

After 2 h at 22° C., the reaction mixture was loaded onto a HiLoad 16/600 Superdex 200 pg column. Elution was carried out with DPBS, pH 7.5 buffer, 10% IPA and a constant flow of 1.0 mL/min. Fractions with a monomeric purity>95% with no unconjugated antibody were pooled and sterile filtered through a 0.22 μm pore size, PVDF membrane filter. The final conjugate sample (7.1 mg; 1.8 mL) was obtained.

The SN-38 reagent (11) hIgG1 isotype control ADC was characterised by HIC, SEC and quantified by UV and endotoxin levels were determined (analytical results shown in Table 6).

TABLE 6
Analytical summary of SN-38 reagent (11) hIgG1 isotype control ADC

| Analysis | Results |
|---|---|
| DAR variants (HIC) | DAR 4: 7.2% |
| | DAR 8: 35.6% |
| | DAR 16: 34.5% |
| | DAR 24: 17.5% |
| | DAR 32: 5.1% |
| | Average DAR: 18.6 |
| Average DAR (UV) | Average DAR: 18.9 |
| % Purity (SEC) | 96.8% monomeric |
| Amount (by UV Analysis) | 7.1 mg |

### Example 13

### Cell Viability Assay with SN-38 ADCs

The CellTiter-Glo® luminescence viability assay (Promega, Southampton, UK) was used to measure the inhibitory effect of the SN-38 ADCs on cell growth. Any reduction in cell proliferation or metabolic activity is indicative of the cytotoxic and/or cytostatic properties of a compound. SK-BR-3 cells (human breast adenocarcinoma, ATCC HTB-30) were cultured in McCoys 5A media (ThermoFisher Scientific, Loughborough, UK) supplemented with 200 U/mL penicillin, 200 μg/mL streptomycin and 20% heat-inactivated fetal bovine serum (Cytiva Hyclone™, ThermoFisher Scientific, Loughborough, UK). SK-BR-3 (HER2 High) cells were seeded in 384-well plates at a density of 1.25×10³ cells in 20 μL growth medium. 3×384 well plates were prepared for each cell line to allow for the incubation timepoints. These were then incubated for 24 hours at 37° C., 5% CO₂. After 24 hours, 20 μL 2× serial dilutions of test samples in growth medium was added.

Each sample was added in triplicate, and the plates were then incubated for 9 hours (limited exposure) or 96 hours (continuous exposure) at 37° C./5% CO₂. After 9 hours, the limited exposure treated plates were removed from the incubator and media containing compound was removed. Cells were washed 2× with growth medium and 40 μL growth medium was then added to each well. Plates were incubated at 37° C./5% CO₂ for a further 96 hours.

Viability was detected using the CellTiter-Glo® luminescence assay. Assay plates were equilibrated at room temperature for 20 minutes before addition of 40 μL CellTiter-Glo® reagent (prepared according to supplier's recommendation) per well. The plates were then mixed for 3 minutes at 300 rpm to assist cell lysis and incubated for a further 20 minutes at room temperature to stabilise the luminescence signal. Luminescence was recorded using a SpectraMax i3x plate reader (Molecular Devices, Wokingham, UK), with a default integration time of 0.5 s/well. Viability data was collected at the timepoints via the same procedure.

Data was then analysed on GraphPad Prism version 8 (GraphPad Software, La Jolla, Calif.) using a four-parameter non-linear regression model. Viability was expressed as a percentage of untreated cells, 100% viability corresponding to the average luminescence of wells containing cells treated with complete medium only. The % viability (Y-axis) was plotted against the total test compounds in M (X-axis) and the software was used to calculate the IC₅₀ values for all ADCs and free drugs.

Cell assay included SN-38 reagent (11) ADC, SN-38 reagent (13) ADC, two control ADCs—(a) trastuzumab conjugated to CL2A-SN-38 at DAR 8 ADC (named Trastuzumab-CL2A-SN-38), and (b) IgG1 isotype control ADC with SN-38 reagent (11) (named Isotype ADC)—and SN-38 free drug.

TABLE 7
Summary of the anti-proliferative effect (IC₅₀ values) of
SN-38 reagent (11) ADC, SN-38 reagent (13) ADC, isotype
ADC, Trastuzumab-CL2A-SN-38, and free payload
SN-38 incubated 9 h and 96 h on SK-BR-3 cells (n = 3).

| | SK-BR-3 IC₅₀ (nM) | |
|---|---|---|
| | Mean (±St.Dev.) | |
| | 96 h | 9 h |
| Sample | exposure | exposure |
| SN-38 reagent (11) ADC | 0.04 ± 0.01 | 0.68 ± 0.57 |
| SN-38 reagent (13) ADC | 0.04 ± 0.01 | 0.18 ± 0.15 |
| Isotype ADC | 0.11 ± 0.01 | 12.1 ± 8.81 |
| Trastuzumab-CL2A-SN-38 | 0.1 ± 0.01 | 5.56 ± 2.91 |
| SN-38 | 1.17 ± 0.09 | 5.78 ± 2.01 |

Due to spontaneous release of SN-38 from the ADCs, the cytotoxic effect of the ADCs and free SN-38 on the tumour cells was determined using limited (9 h) as well as continuous exposure (96 h) assays. Limited exposure assays (cytotoxic compounds were removed following 9-hour incubation with cells) overall showed lower background cytotoxicity in cultures treated with the ADC isotype control compared to SN-38 reagent (11) ADC and SN-38 reagent (13) ADC (Table 7). In addition, the limited exposure data indicates that the SN-38 reagent (11) ADC and SN-38 reagent (13) ADC are more potent in inducing cell death in SK-BR-3 cells compared to the Trastuzumab-CL2A-SN-38 (Table 7).

### Example 14

### Serum Stability of SN-38 ADCs

The aim of this study was to monitor the stability of SN-38 reagent (11) ADC and SN-38 reagent (13) ADC and control ADC trastuzumab conjugated to CL2A-SN-38 at DAR 8 (Trastuzumab-CL2A-SN-38) in mouse plasma, over 96 hours incubation at 37° C.

ADCs were spiked into mouse plasma and incubated at 37° C. over a 96 h period. To evaluate the changes in DAR profile throughout plasma incubation, ADCs were analysed by HIC-UV (214 nm) after isolation from plasma using affinity capture.

Higher stability was observed for SN-38 reagent (11) ADC and SN-38 reagent (13) ADC compared to control ADC Trastuzumab-CL2A-SN-38. For SN-38 reagent (11) ADC and SN-38 reagent (13) ADC, a progressive decrease in higher DAR species and increase in lower DAR species is observed for later time points, with an approx. 50-55% decrease of average DAR after 96 hours. For Trastuzumab-CL2A-SN-38, a major decrease in high DAR species was observed after 48 hours incubation in mouse plasma, displaying a lower stability in mouse plasma, with more than 70% decrease of high DAR species after 48 hours.

### Example 15

### Serum Stability of MMAE ADC

The aim of this study was to monitor the stability of MMAE ADC (prepared in Example 3) and control ADC trastuzumab conjugated to MC-VC-PAB-MMAE (named Trastuzumab-MC-VC-PAB-MMAE) in mouse plasma, over 96 hours incubation at 37° C.

ADCs were spiked into mouse serum and incubated at 37° C. over a 96-hour period. To evaluate the changes in DAR profile throughout serum incubation, ADCs were analysed by HIC-UV (280 nm) after being isolated from serum using affinity capture.

Higher stability, approx. 16% DAR loss over a 96-hour period, was observed for the ADC, upon incubation in mouse serum for 96 h, compared to control ADC Trastuzumab-MC-VC-PAB-MMAE with approx. 44% DAR loss a 96-hour period.

1. An antibody-drug conjugate comprising:
an antibody or antigen-binding fragment thereof;
(ii) a polymer comprising a repeat unit of Formula (I): 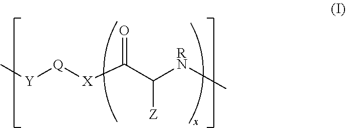
wherein:
X is selected from O, NH, NR^{A} and S;
Y is selected from C═O, C═NH, C═NR^{A} and C═S;
R is hydrogen or C₁₋₂₀ hydrocarbyl;
R^{A} is C₁₋₂₀ hydrocarbyl;
each Q is independently selected from —CH₂(NMe(C═O)CH₂)ₒ—, -T¹O(CH₂C₂O)ₛT²- and -T¹O(CH₂CH₂C₂O)ₛT²-, wherein T¹ is selected from a divalent methylene, ethylene, propylene or butylene radical, and T² is selected from a divalent methylene, ethylene, propylene or butylene radical;
o is an integer from 0 to 100;
s is an integer from 0 to 150;
x is an integer from 1 to 6; and
each Z is independently selected from a group of formula (i), (ii), (iii), (iv) or (v): 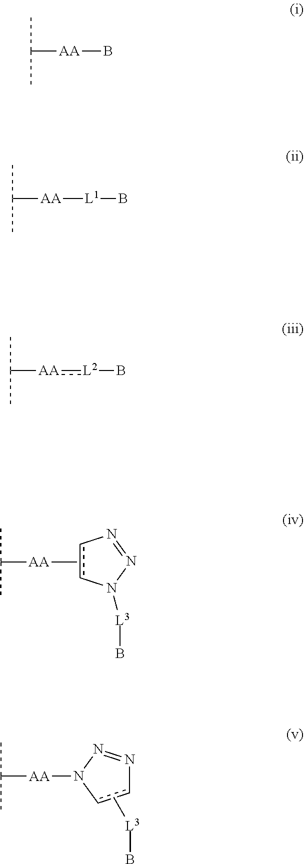
wherein,
when Z is a group of formula (i) or (ii):
-AA- is a divalent moiety such that -AA-H represents the side chain of an amino acid;
each L¹ is a linker group; and
each B is a biologically active moiety;
when Z is a group of formula (iii):
-AA= is a trivalent moiety such that -AA=O represents the side chain of an amino acid;
each L² is a linker group;
each dashed line represents a bond which is either present or absent; and
each B is a biologically active moiety;
when Z is a group of formula (iv):
-AA- is a divalent moiety such that -AA-CH═CH₂ or -AA-CCH represents the side chain of an amino acid;
each L³ is a linker group;
each dashed line represents a bond which is either present or absent; and
each B is a biologically active moiety; and when Z is a group of formula (v):
-AA- is a divalent moiety such that -AA-N₃ represents the side chain of an amino acid;
each L³ is a linker group;
each dashed line represents a bond which is either present or absent; and
each B is a biologically active moiety; and
(iii) a polymer-antibody linker which is covalently bonded to both the antibody and the polymer.

2. An antibody-drug conjugate according to claim 1, wherein the group of formula (ii) is a group of formula (vi): 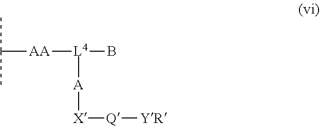
and/or the group of formula (iii) is a group of formula (vii): 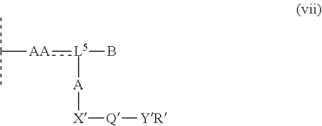
and/or the group of formula (iv) is a group of formula (viii): 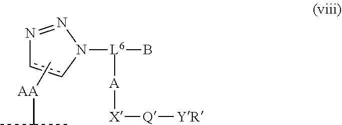
and/or the group of formula (v) is a group of formula (ix): 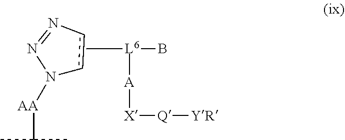
wherein:
each L⁴ is a linker group;
each L⁵ is a linker group;
each L⁶ is a linker group;
each A is independently selected from a bond, an amino acid, a peptide, a sulfonate, a sulfonamide, or a pyrophosphate diester;
each X′ is independently selected from O, NH, NR^{A′} and S;
each R′ is independently hydrogen or C₁₋₂₀ hydrocarbyl;
each R^{A′} is independently C₁₋₂₀ hydrocarbyl;
each Q′ is independently selected from —CH₂(NMe(C═O)CH₂)_{o′}—, -T′¹O(CH₂C₂O)_{s′}T′²- and -T′¹O(CH₂CH₂C₂O)_{s′}T′²-, wherein each T′¹ is independently selected from a divalent methylene, ethylene, propylene or butylene radical, and each T′² is independently selected from a divalent methylene, ethylene, propylene or butylene radical, wherein the left-hand side of the Q′ moiety as drawn is covalently bonded to the Y′ moiety, and the right-hand side of the Q′ moiety as drawn is covalently bonded to the X′ moiety;
each dashed line represents a bond which is either present or absent;
each o′ is independently an integer from 0 to 100; and
each s′ is independently an integer from 0 to 150;
when Q′ is -T′¹O(CH₂C₂O)_{s′}T′²- and -T′¹O(CH₂CH₂C₂O)_{s′}T′²-, each Y′ is independently selected from O, NH, NR^{A′} and S, and when Q′ is —CH₂(NMe(C═O)CH₂)_{o′}—, each Y′ is independently selected from —(C═O)—O—, —(C═O)—S—, —(C═O)—NH and —(C═O)—NR^{A′}—.

3. An antibody-drug conjugate according to claim 1, wherein:
(a) -AA-H represents the side chain of an amino acid selected from serine, cysteine, threonine, asparagine, glutamine, aspartic acid, glutamic acid, lysine, arginine, tyrosine, tryptophan, histidine, ornithine, hydroxytryptophan, homoserine, homocysteine, allothreonine, selenocysteine, selenohomocysteine, α-aminoglycine, diaminoacetic acid, 2,3-diaminopropionic acid and α,γ-diaminobutyric acid, preferably the side chain of an amino acid selected from serine, cysteine, threonine, lysine and ornithine, and most preferably the side chain of lysine; or
(b) -AA=O represents the side chain of an amino acid selected from amino-2-keto-butyric acid, 4-acetylphenylalanine and formylglycine; or
(c) -AA-N₃ represents the side chain of an amino acid selected from azidolysine, azidoornithine, azidonorleucine, azidoalanine, azidohomoalanine, 4-azidophenylalanine and 4-azidomethylphenylalanine; or
(d) -AA-CH═CH₂ represents the side chain of homoallylglycine; or
(e) -AA-C≡CH represents the side chain of an amino acid selected from 4-ethynylphenylalanine, 4-propargyloxyphenylalanine, propargylglycine, 4-(2-propynyl)proline, 2-amino-6-({[(1R,8S)-bicyclo[6.1.0]non-4-yn-9-ylmethoxy]carbonyl}amino)hexanoic acid and homopropargylglycine.

4. An antibody-drug conjugate according to claim 1, wherein B in formula (i), L¹ in formula (ii) and/or L⁴ in formula (vi) is covalently bound to the moiety AA through a heteroatom in the amino acid side chain.

5. An antibody-drug conjugate according to claim 1, wherein the polymer-antibody linker:
(i) is covalently bound to the polymer through the nitrogen atom of the —NR— group in Formula (I) or the Y group in Formula (I); and/or
(ii) is derived from maleimide, monobromomaleimide, vinyl sulfones, bis(sulfone)s, allenamides, dehydroalanine, alkenes, perfluoroaromatic species, sulfone reagents that are Julia-Kocienski like, N-hydroxysuccinamide-ester activated carboxylate species, aldehydes, ketones, hydroxylamines, alkynes and azides.

6. (canceled)

7. An antibody-drug conjugate according to claim 1, wherein X is O or NH and Y is C═O.

8. An antibody-drug conjugate according to claim 2, wherein Z is a group of formula (vi), (vii), (viii) or (ix) and X′ is O or NH and Y′ is O or NH, preferably wherein X′ is NH and Y′ is O.

9. An antibody-drug conjugate according to claim 1, wherein Q is —CH₂CH₂O(CH₂CH₂O)ₛCH₂CH₂— or —CH₂CH₂CH₂O(CH₂C₂O)ₛCH₂CH₂CH₂—, preferably wherein s is from 1 to 100, preferably wherein Q is —CH₂CH₂O(CH₂CH₂O)ₛCH₂C₂— and s is 3, 7, 11, 23 or 35.

10. (canceled)

11. An antibody-drug conjugate according to claim 2, wherein Z is a group of formula (vi), (vii), (viii) or (ix) and Q′ is —CH₂CH₂O(CH₂CH₂O)ₛCH₂CH₂— or —CH₂CH₂CH₂O(CH₂CH₂O)ₛCH₂CH₂CH₂—, preferably wherein s is from 1 to 100, preferably wherein Z is a group of formula (vi), (vii), (viii) or (ix) and Q is —CH₂CH₂O(CH₂CH₂O)ₛCH₂C₂— and s is 3, 7, 11, 23 or 35.

12. (canceled)

13. An antibody-drug conjugate according to claim 2, wherein Z is a group of formula (vi), (vii), (viii) or (ix) and R′ is selected from hydrogen and C₁₋₆ alkyl, preferably wherein R′ is hydrogen, methyl, ethyl or n-propyl.

14. An antibody-drug conjugate according to claim 1, wherein each biologically active moiety —B is the same or different, such that each B—H or B—OH is independently selected from small molecule drugs, peptides, proteins, peptide mimetics, antibodies, antigens, DNA, mRNA, small interfering RNA, small hairpin RNA, microRNA, PNA, foldamers, carbohydrates, carbohydrate derivatives, non-Lipinski molecules, synthetic peptides and synthetic oligonucleotides, preferably small molecule drugs.

15. An antibody-drug conjugate according to claim 1, wherein:
(a) Z is a group of formula (ii) and L¹ is a linker moiety of formula —V-L′-V²—,
wherein:
V¹ is selected from 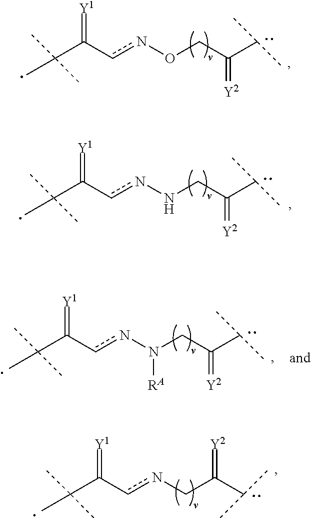
wherein
• denotes the point of attachment to -AA-;
•• denotes the point of attachment to -L′-;
Y¹ is selected from O, S and NH, and is preferably O;
Y² is selected from O, S and NH, and is preferably O;
R^{A} is C₁₋₂₀ hydrocarbyl;
v is an integer from 1 to 100, preferably from 1 to 10; and
a dashed line represents an optionally present bond;
L′ is selected from a bond, C₁₋₂₀ alkylene, C₁₋₂₀ alkenylene, C₁₋₂₀ alkynylene, C₆₋₁₀ arylene (e.g. phenylene or naphthylene), C₇₋₂₀ aralkylene, C₃₋₁₀ cycloalkylene, C₄₋₈ heterocycloalkylene, C₅₋₁₀ heteroarylene, C₆₋₂₀ heteroaralkylene, —(O—K)ᵢ—, —(NH—K)ᵢ—, —(NR′—K)ᵢ—, a polyester having a molecular weight of from 116 to 2000 Da, a polyamide having a molecular weight of from 114 to 2000 Da, and a moiety —W— wherein H—W—OH is an amino acid or a peptide containing from two to twenty naturally-occurring or synthetic amino acid subunits;
V² is selected from —OV—, —NHV—, —NR^{A}V—, —SV—, —S—, —VS—, —OVS—, —NHVS—, —NR^{A}VS—, —SVS—, —V—(C═O)—, —V—O(C═O)—, —V—NH(C═O)—, —V—NR^{A}(C═O)—, —V—S(C═O)—, —V—(C═NH)—, —V—O(C═NH)—, —V—NH(C═NH)—, —V—NR^{A}(C═NH)—, —V—S(C═NH)—, —V—(C═NR^{A})—, —V—O(C═NR^{A})—, —V—NH(C═NR^{A})—, —V—NR^{A}(C═NR^{A})—, —V—S(C═NR^{A})—, —OV—(C═O)—, —OV—O(C═O)—, —OV—NH(C═O)—, —OV—NR^{A}(C═O)—, —OV—S(C═O)—, —OV—(C═NH)—, —OV—O(C═NH)—, —OV—NH(C═NH)—, —OV—NR^{A}(C═NH)—, —OV—S(C═NH)—, —OV—(C═NR^{A})—, —OV—O(C═NR^{A})—, —OV—NH(C═NR^{A})—, —OV—NR^{A}(C═NR^{A})—, —OV—S(C═NR^{A})—, —NHV—(C═O)—, —NHV—O(C═O)—, —NHV—NH(C═O)—, —NHV—NR^{A}(C═O)—, —NHV—S(C═O)—, —NHV—(C═NH)—, —NHV—O(C═NH)—, —NHV—NH(C═NH)—, —NHV—NR^{A}(C═NH)—, —NHV—S(C═NH)—, —NHV—(C═NR^{A})—, —NHV—O(C═NR^{A})—, —NHV—NH(C═NR^{A})—, —NHV—NR^{A}(C═NR^{A})—, —NHV—S(C═NR^{A})—, —NR^{A}V—(C═O)—, —NR^{A}V—O(C═O)—, —NR^{A}V—NH(C═O)—, —NR^{A}V—NR^{A}(C═O)—, —NR^{A}V—S(C═O)—, —NR^{A}V—(C═NH)—, —NR^{A}V—O(C═NH)—, —NR^{A}V—NH(C═NH)—, —NR^{A}V—NR^{A}(C═NH), —NR^{A}V—S(C═NH)—, —NR^{A}V—(C═NR^{A})—, —NR^{A}V—O(C═NR^{A})—, —NR^{A}V—NH(C═NR^{A})—, —NR^{A}V—NR^{A}(C═NR^{A})—, —NR^{A}V—S(C═NR^{A})—, —SV—(C═O)—, —SV—O(C═O)—, —SV—NH(C═O)—, —SV—NR^{A}(C═O)—, —SV—S(C═O)—, —SV—(C═NH)—, —SV—O(C═NH)—, —SV—NH(C═NH)—, —SV—NR^{A}(C═NH)—, —SV—S(C═NH)—, —SV—(C═NR^{A})—, —SV—O(C═NR^{A})—, —SV—NH(C═NR^{A})—, —SV—NR^{A}(C═NR^{A})—, —SV—S(C═NR^{A})—, -J-O(C═O)—, —O-J-O(C═O)—, —S-J-O(C═O)—, —NH-J-O(C═O)—, —NR^{A}-J-O(C═O)—, a polyether e.g. poly(alkylene glycol) having a molecular weight of from 76 to 2000 Da, a polyamine having a molecular weight of from 75 to 2000 Da, a polyester having a molecular weight of from 116 to 2000 Da, a polyamide having a molecular weight of from 114 to 2000 Da, and a moiety —W— wherein H—W—OH is an amino acid or a peptide containing from two to twenty naturally-occurring or synthetic amino acid subunits;
V is selected from C₁₋₂₀ alkylene, C₁₋₂₀ alkenylene, C₁₋₂₀ alkynylene, C₆₋₁₀ arylene (e.g. phenylene or naphthylene), C₇₋₂₀ aralkylene, C₃₋₁₀ cycloalkylene, C₄₋₈ heterocycloalkylene, C₅₋₁₀ heteroarylene, and C₆₋₂₀ heteroaralkylene;
J is a phenyl group which carries a sugar substituent and, para or ortho to the sugar substituent, a methylene group or a moiety —(CH═CH)ₖ—CH₂—, wherein k is an integer from 1 to 10, further wherein the methylene group or moiety
—(CH═CH)ₖ—CH₂— is directly bonded to the —O(C═O)— group proximal to the biologically active moiety B, and a carbon of the phenyl ring is directly bonded to the remainder of the linker group distal to the biologically active moiety B;
each K is the same or different and represents C₁₋₁₀ alkylene;
i is an integer from 1 to 100, preferably from 1 to 50, and more preferably from 2 to 20; and
R^{A} is C₁₋₂₀ hydrocarbyl;
preferably wherein L¹ is a moiety selected from —(C═O)—C(H)═N—NH—CH₂—(C═O)-Val-Cit-PAB-(C═O)—, —(C═O)—C(H)═N—O—CH₂—(C═O)-Val-Cit-PAB-(C═O)—, —(C═O)—C(H)═N—CH₂—(C═O)-Val-Cit-PAB-(C═O)—, —(C═O)—CH₂—NH—NH—CH₂—(C═O)-Val-Cit-PAB-(C═O)—, —(C═O)—CH₂—NH—O—CH₂—(C═O)-Val-Cit-PAB-(C═O)— and —(C═O)—CH₂—NH—CH₂—(C═O)-Val-Cit-PAB-(C═O)—; or
(b) Z is group of formula (vi) and L⁴ is a linker moiety of formula (x) or (xi): 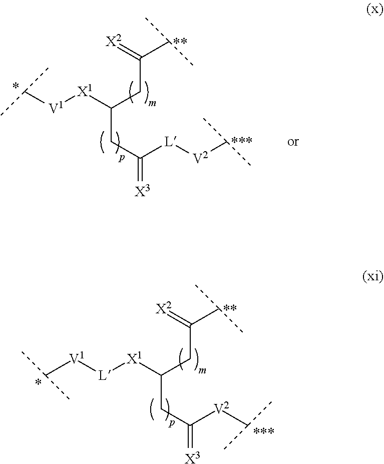
wherein:
* denotes the point of attachment to -AA-;
** denotes the point of attachment to -A-X′-Q′-Y′R′;
*** denotes the point of attachment to —B;
V¹, L′ and V² are as defined in (a) above;
X¹ is selected from O, S and NH;
X² is selected from O, S and NH;
X³ is selected from O, S and NH;
R^{A} is C₁₋₂₀ hydrocarbyl;
m is an integer from 0 to 6; and
p is an integer from 0 to 6.

16. An antibody-drug conjugate according to claim 1, wherein:
(a) Z is a group of formula (iii) and L² is a linker moiety of formula ═V³-L′-V²—, wherein:
V³ is selected from 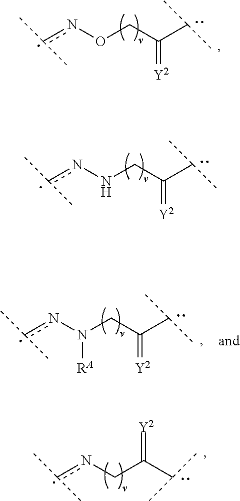
wherein
• denotes the point of attachment to -AA-;
•• denotes the point of attachment to -L′-;
Y² is selected from O, S and NH, and is preferably O;
R^{A} is C₁₋₂₀ hydrocarbyl;
v is an integer from 1 to 100, preferably from 1 to 10; and
a dashed line represents an optionally present bond;
L′ is selected from a bond, C₁₋₂₀ alkylene, C₁₋₂₀ alkenylene, C₁₋₂₀ alkenylene, C₆₋₁₀ arylene (e.g. phenylene or naphthylene), C₇₋₂₀ aralkylene, C₃₋₁₀ cycloalkylene, C₄₋₈ heterocycloalkylene, C₅₋₁₀ heteroarylene, C₆₋₂₀ heteroaralkylene, —(O—K)ᵢ—, —(NH—K)ᵢ—, —(NR′—K)ᵢ—, a polyester having a molecular weight of from 116 to 2000 Da, a polyamide having a molecular weight of from 114 to 2000 Da, and a moiety —W— wherein H—W—OH is an amino acid or a peptide containing from two to twenty naturally-occurring or synthetic amino acid subunits; and
V² is selected from —OV—, —NHV—, —SV—, —S—, —VS—, —OVS—, —NHVS—, —NR^{A}VS—, —SVS-, —V—(C═O)—, —V—O(C═O)—, —V—NH(C═O)—, —V—NR^{A}(C═O)—, —V—S(C═O)—, —V—(C═NH)—, —V—O(C═NH)—, —V—NH(C═NH)—, —V—NR^{A}(C═NH)—, —V—S(C═NH)—, —V—(C═NR^{A})—, —V—O(C═NR^{A})—, —V—NH(C═NR^{A})—, —V—NR^{A}(C═NR^{A})—, —V—S(C═NR^{A})—, —OV—(C═O)—, —OV—O(C═O)—, —OV—NH(C═O)—, —OV—NR^{A}(C═O)—, —OV—S(C═O)—, —OV—(C═NH)—, -OV—O(C═NH)—, —OV—NH(C═NH)—, —OV—NR^{A}(C═NH)—, —OV—S(C═NH)—, —OV—(C═NR^{A})—, —OV—O(C═NR^{A})—, —OV—NH(C═NR^{A})—, —OV—NR^{A}(C═NR^{A})—, —OV—S(C═NR^{A})—, —NHV—(C═O)—, —NHV—O(C═O)—, —NHV—NH(C═O)—, —NHV—NR^{A}(C═O)—, —NHV—S(C═O)—, —NHV—(C═NH)—, —NHV—O(C═NH)—, —NHV—NH(C═NH)—, —NHV—NR^{A}(C═NH)—, —NHV—S(C═NH)—, —NHV—(C═NR^{A})—, —NHV—O(C═NR^{A})—, —NHV—NH(C═NR^{A})—, —NHV—NR^{A}(C═NR^{A})—, —NHV—S(C═NR^{A})—, —NR^{A}V—(C═O)—, —NR^{A}V—O(C═O)—, —NR^{A}V—NH(C═O)—, —NR^{A}V—NR^{A}(C═O)—, —NR^{A}V—S(C═O)—, —NR^{A}V—(C═NH)—, —NR^{A}V—O(C═NH)—, —NR^{A}V—NH(C═NH)—, —NR^{A}V—NR^{A}(C═NH)—, —NR^{A}V—S(C═NH)—, —NR^{A}V—(C═NR^{A})—, —NR^{A}V—O(C═NR^{A})—, —NR^{A}V—NH(C═NR^{A})—, —NR^{A}V—NR^{A}(C═NR^{A})—, —NR^{A}V—S(C═NR^{A})—, —SV—(C═O)—, —SV—O(C═O)—, —SV—NH(C═O)—, —SV—NR^{A}(C═O)—, —SV—S(C═O)—, —SV—(C═NH)—, —SV—O(C═NH)—, —SV—NH(C═NH)—, —SV—NR^{A}(C═NH)—, —SV—S(C═NH)—, —SV—(C═NR^{A})—, —SV—O(C═NR^{A})—, —SV—NH(C═NR^{A})—, —SV—NR^{A}(C═NR^{A})—, —SV—S(C═NR^{A})—, -J-O(C═O)—, —O-J-O(C═O)—, —S-J-O(C═O)—, —NH-J-O(C═O)—, —NR^{A}-J-O(C═O)—, a polyether e.g. poly(alkylene glycol) having a molecular weight of from 76 to 2000 Da, a polyamine having a molecular weight of from 75 to 2000 Da, a polyester having a molecular weight of from 116 to 2000 Da, a polyamide having a molecular weight of from 114 to 2000 Da, and a moiety —W— wherein H—W—OH is an amino acid or a peptide containing from two to twenty naturally-occurring or synthetic amino acid subunits;
preferably wherein L² is a moiety selected from ═N—NH—CH₂—(C═O)-Val-Cit-PAB-(C═O)—, ═N—O—CH₂—(C═O)-Val-Cit-PAB-(C═O)—, ═N—CH₂—(C═O)-Val-Cit-PAB-(C═O)—, —NH—NH—CH₂—(C═O)-Val-Cit-PAB-(C═O)—, —NH—O—CH₂—(C═O)-Val-Cit-PAB-(C═O)— and —NH—CH₂—(C═O)-Val-Cit-PAB-(C═O)—; or
(b) Z is group of formula (vii) and L⁵ is a linker moiety of formula (xii) or (xiii): 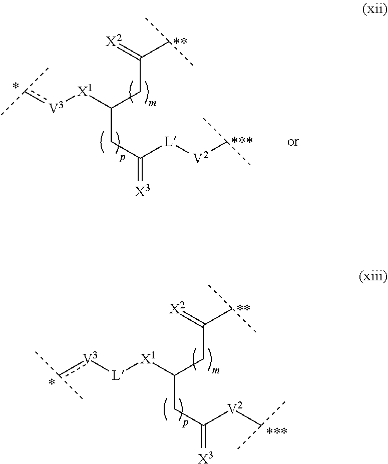
wherein
* denotes the point of attachment to -AA-;
** denotes the point of attachment to -A-X′-Q′-Y′R′;
*** denotes the point of attachment to —B;
L′ is as defined in (a) above;
V² is defined in (a) above;
X¹ is selected from O, S and NH;
X² is selected from O, S and NH;
X³ is selected from O, S and NH;
R^{A} is C₁₋₂₀ hydrocarbyl;
m is an integer from 0 to 6;
p is an integer from 0 to 6; and
V³ is as defined in (a) above, and a dashed line is a bond that can be present or absent.

17. An antibody-drug conjugate according to claim 1, wherein:
(a) Z is a group of formula (iv) or (v) and L³ is a linker moiety of formula —V⁴-L′-V²—, wherein:
V⁴ is —(CH₂)ᵥ—(C═Y²), wherein
Y² is selected from O, S and NH, and is preferably O; and
v is an integer from 1 to 100, preferably from 1 to 10;
L′ is selected from a bond, C₁₋₂₀ alkylene, C₁₋₂₀ alkenylene, C₁₋₂₀ alkynylene, C₆₋₁₀ arylene (e.g. phenylene or naphthylene), C₇₋₂₀ aralkylene, C₃₋₁₀ cycloalkylene, C₄₋₈ heterocycloalkylene, C₅₋₁₀ heteroarylene, C₆₋₂₀ heteroaralkylene, —(O—K)ᵢ—, —(NH—K)ᵢ—, —(NR′—K)ᵢ—, a polyester having a molecular weight of from 116 to 2000 Da, a polyamide having a molecular weight of from 114 to 2000 Da, and a moiety —W— wherein H—W—OH is an amino acid or a peptide containing from two to twenty naturally-occurring or synthetic amino acid subunits; and
V² is selected from —OV—, —NHV—, —NR^{A}V—, —SV—, —S—, —VS—, —OVS—, —NHVS—, —NR^{A}VS—, —SVS—, —V—(C═O)—, —V—O(C═O)—, —V—NH(C═O)—, —V—NR^{A}(C═O)—, —V—S(C═O)—, —V—(C═NH)—, —V—O(C═NH)—, —V—NH(C═NH)—, —V—NR^{A}(C═NH)—, —V—S(C═NH)—, —V—(C═NR^{A})—, —V—O(C═NR^{A})—, —V—NH(C═NR^{A})—, —V—NR^{A}(C═NR^{A})—, —V—S(C═NR^{A})—, —OV—(C═O)—, —OV—O(C═O)—, —OV—NH(C═O)—, —OV—NR^{A}(C═O)—, —OV—S(C═O)—, —OV—(C═NH)—, —OV—O(C═NH)—, —OV—NH(C═NH)—, —OV—NR^{A}(C═NH)—, —OV—S(C═NH)—, —OV—(C═NR^{A})—, —OV—O(C═NR^{A})—, —OV—NH(C═NR^{A})—, —OV—NR^{A}(C═NR^{A})—, —OV—S(C═NR^{A})—, —NHV—(C═O)—, —NHV—O(C═O)—, —NHV—NH(C═O)—, —NHV—NR^{A}(C═O)—, —NHV—S(C═O)—, —NHV—(C═NH)—, —NHV—O(C═NH)—, —NHV—NH(C═NH)—, —NHV—NR^{A}(C═NH)—, —NHV—S(C═NH)—, —NHV—(C═NR^{A})—, —NHV—O(C═NR^{A})—, —NHV—NH(C═NR^{A})—, —NHV—NR^{A}(C═NR^{A})—, —NHV—S(C═NR^{A})—, —NR^{A}V—(C═O)—, —NR^{A}V—O(C═O)—, —NR^{A}V—NH(C═O)—, —NR^{A}V—NR^{A}(C═O)—, —NR^{A}V—S(C═O)—, —NR^{A}V—(C═NH)—, —NR^{A}V—O(C═NH)—, —NR^{A}V—NH(C═NH)—, —NR^{A}V—NR^{A}(C═NH)—, —NR^{A}V—S(C═NH)—, —NR^{A}V—(C═NR^{A})—, —NR^{A}V—O(C═NR^{A})—, —NR^{A}V—NH(C═NR^{A})—, —NR^{A}V—NR^{A}(C═NR^{A})—, —NR^{A}V—S(C═NR^{A})—, —SV—(C═O)—, —SV—O(C═O)—, —SV—NH(C═O)—, —SV—NR^{A}(C═O)—, —SV—S(C═O)—, —SV—(C═NH)—, —SV—O(C═NH)—, —SV—NH(C═NH)—, —SV—NR^{A}(C═NH)—, —SV—S(C═NH)—, —SV—(C═NR^{A})—, —SV—O(C═NR^{A})—, —SV—NH(C═NR^{A})—, —SV—NR^{A}(C═NR^{A})—, —SV—S(C═NR^{A})—, -J-O(C═O)—, —O-J-O(C═O)—, —S-J-O(C═O)—, —NH-J-O(C═O)—, —NR^{A}-J-O(C═O)—, a polyether e.g. poly(alkylene glycol) having a molecular weight of from 76 to 2000 Da, a polyamine having a molecular weight of from 75 to 2000 Da, a polyester having a molecular weight of from 116 to 2000 Da, a polyamide having a molecular weight of from 114 to 2000 Da, and a moiety —W— wherein H—W—OH is an amino acid or a peptide containing from two to twenty naturally-occurring or synthetic amino acid subunits;
(b) Z is group of formula (viii) or (ix) and L⁶ is a linker moiety of formula (xii) or (xiii): 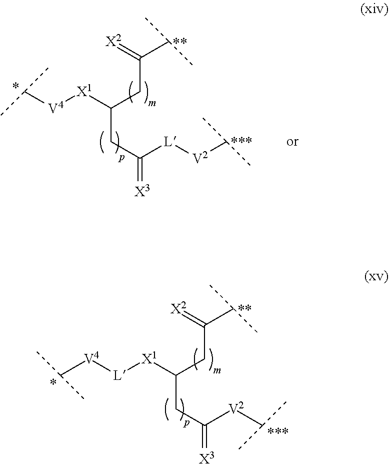
* denotes the point of attachment to -AA-;
** denotes the point of attachment to -A-X′-Q′-Y′R′;
*** denotes the point of attachment to —B;
L′ is as defined in (a) above;
V² is defined in (a) above;
X¹ is selected from O, S and NH;
X² is selected from O, S and NH;
X³ is selected from O, S and NH;
R^{A} is C₁₋₂₀ hydrocarbyl;
m is an integer from 0 to 6;
p is an integer from 0 to 6; and
V⁴ is as defined in (a) above.

18. An antibody-drug conjugate according to claim 15, wherein X¹ is NH, X² is O, X³ is O, preferably wherein one of m and p is either 2 or 3, and the other is 0.

19. An antibody-drug conjugate according to claim 1 having Formula (III) or (IV): 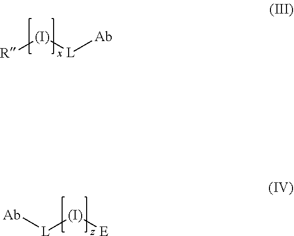
wherein:
(I) is a repeat unit of the Formula (I);
Ab is an antibody or antigen-binding fragment thereof;
L is a polymer-antibody linker;
R″ is selected from OH, OR^{A}, SH, SR^{A}, NH₂, NHR^{A} and NR^{A}₂;
E is selected from H and R^{A}; and
z is an integer from 1 to 50.

20. A pharmaceutical composition comprising an antibody-drug conjugate according to claim 1 and a pharmaceutically acceptable excipient.

21. (canceled)

22. A method of treating a disease or condition in a human patient, wherein said disease or condition is selected from inflammatory diseases (e.g. inflammatory bowel disease, rheumatoid arthritis and artherosclerosis), metabolic disorders (e.g. diabetes, insulin resistance, obesity), cancer, bacterial infections (e.g. tuberculosis, pneumonia, endocarditis, septicaemia, salmonellosis, typhoid fever, cystic fibrosis, chronic obstructive pulmonary diseases), viral infections, cardiovascular diseases, neurodegenerative diseases, neurological disorders, behavioral and mental disorders, blood diseases, chromosome disorders, congenital and genetic diseases, connective tissue diseases, digestive diseases, ear, nose, and throat diseases, endocrine diseases, environmental diseases, eye diseases, female reproductive diseases, fungal infections, heart diseases, hereditary cancer syndromes, immune system diseases, kidney and urinary diseases, lung diseases, male reproductive diseases, mouth diseases, musculoskeletal diseases, myelodysplastic syndromes, nervous system diseases, newborn screening, nutritional diseases, parasitic diseases, rare cancers and skin diseases, and wherein said method comprises administration of at least one antibody-drug conjugate according to claim 1 to a patient in need thereof.

23. (canceled)

24. A targeting agent-drug conjugate comprising:
(i) a targeting agent;
(ii) a polymer comprising a repeat unit of Formula (I): 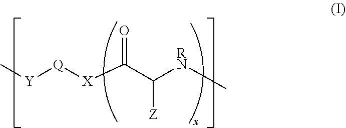
wherein:
X is selected from O, NH, NR^{A} and S;
Y is selected from C═O, C═NH, C═NR^{A} and C═S;
R is hydrogen or C₁₋₂₀ hydrocarbyl;
R^{A} is C₁₋₂₀ hydrocarbyl;
each Q is independently selected from —CH₂(NMe(C═O)CH₂)ₒ—, -T¹O(CH₂C₂O)ₛT²- and -T¹O(CH₂CH₂C₂O)ₛT²-, wherein T¹ is selected from a divalent methylene, ethylene, propylene or butylene radical, and T² is selected from a divalent methylene, ethylene, propylene or butylene radical;
o is an integer from 0 to 100;
s is an integer from 0 to 150;
x is an integer from 1 to 6; and
each Z is independently selected from a group of formula (i), (ii), (iii), (iv) or (v): 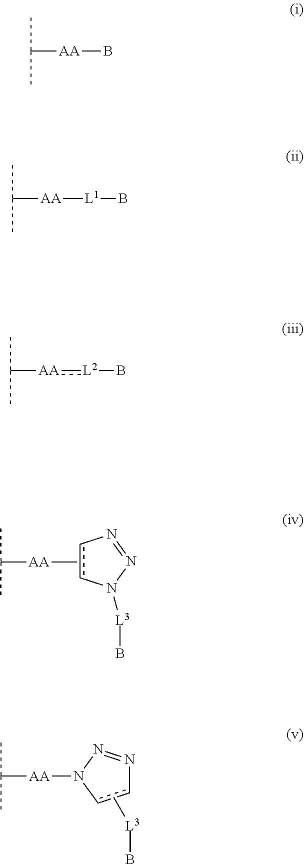
wherein,
when Z is a group of formula (i) or (ii):
-AA- is a divalent moiety such that -AA-H represents the side chain of an amino acid;
each L¹ is a linker group; and
each B is a biologically active moiety;
when Z is a group of formula (iii):
-AA= is a trivalent moiety such that -AA=O represents the side chain of an amino acid;
each L² is a linker group;
each dashed line represents a bond which is either present or absent; and
each B is a biologically active moiety;
when Z is a group of formula (iv):
-AA- is a divalent moiety such that -AA-CH═CH₂ or -AA-CCH represents the side chain of an amino acid;
each L³ is a linker group;
each dashed line represents a bond which is either present or absent; and
each B is a biologically active moiety; and
when Z is a group of formula (v):
-AA- is a divalent moiety such that -AA-N₃ represents the side chain of an amino acid;
each L³ is a linker group;
each dashed line represents a bond which is either present or absent; and
each B is a biologically active moiety; and
(iii) a polymer-targeting agent linker which is covalently bonded to both the targeting agent and the polymer.

25. A targeting agent-drug conjugate according to claim 24, wherein the targeting agent is selected from a peptide, a protein, a peptide mimetic, an antibody, an antigen, DNA, mRNA, small interfering RNA, small hairpin RNA, microRNA, PNA, a foldamer, a carbohydrate, a carbohydrate derivative, a non-Lipinski molecule, a synthetic peptide and a synthetic oligonucleotide.
